(19) United States
(12) Patent Application Publication
(10) Pub. No.: US 2026/0159801 A1
(43) Pub. Date: Jun. 11, 2026

(54) METHODS FOR MODULATING THE FUNCTION OF BIOLOGICAL REGULATORY NETWORKS IN HEALTH AND DISEASE BY EXPLOITING THEIR MEMORY PROPERTIES

(71) Applicant: Trustees of Tufts College, Medford, MA (US)

(72) Inventors: Michael Levin, Medford, MA (US); Surama Biswas, Medford, MA (US)

(21) Appl. No.: 19/182,154

(22) Filed: Apr. 17, 2025

Related U.S. Application Data
(63) Continuation-in-part of application No. 17/648,247, filed on Jan. 18, 2022.
(60) Provisional application No. 63/138,240, filed on Jan. 15, 2021.
(60) Provisional application No. 63/640,497, filed on Apr. 30, 2024.

(51) Int. Cl. C12M 1/36; C12M 1/34; G01N 33/50

(52) U.S. Cl. CPC C12M 41/48; C12M 41/32; C12M 41/36; C12M 41/46; G01N 33/5023; G01N 33/505; G01N 33/5052; G01N 33/5058; G01N 2500/10

(57) ABSTRACT

Disclosed are methods for exploiting memory properties of biological systems such as gene regulatory networks (GNRs). The disclosed methods may be utilized in order to treat diseases and disorders and in order to promote health.

### BACKGROUND

The field of the invention relates to memory properties in biological systems. In particular, the field of the invention relates to memory properties in gene regulatory networks (GRNs), protein networks, and biological pathways and to modulating the function of GRNs, protein networks, and biological pathways based on their memory properties to treat diseases and disorders and promote health.

Many processes in health and disease (e.g., embryogenesis, regeneration, cancer, physiology, etc.) are controlled by networks such as gene regulatory networks (GRNs). As GRNs are a central paradigm for understanding the control of embryonic morphogenesis and adult physiology in health and disease. Many efforts have advanced our understanding of GRNs as dynamical systems and revealed how GRNs settle into specific stable states. However, very little has been done to understand long-term changes in GRN dynamics based on their prior history, or to uncover the sources of plasticity in GRNs which could be exploited biomedically.

Here, the inventors take a computational approach, treating GRNs as akin to neural networks to ask what they can learn from past experience that modifies their future response and dynamics. Specifically, the inventors designate some genes as inputs, others as responses, and show that many real biological GRNs should be capable of learning relationships between inputs in a kind of associative memory. In particular, here the inventors: (i) establish a paradigm for understanding GRNs as computational agents, formalizing the notions of stimuli, response, training, and behavior assays; (ii) produce a taxonomy of learning in GRNs, rigorously defining various types of memories that can exist in GRNs; (iii) produce a new methodology and software suite which automates the discovery of memory types, and thus of powerful intervention strategies for manipulating GRN responses; (iv) conduct a broad survey of biological GRNs across the tree of life, identifying their types of memories; (v) show that biological GRNs have unique memory properties differing from randomized GRNs (suggesting that evolution favors specific memory types in GRNs); and (vi) discuss the evolutionary and biomedical implications of GRN memories, which extend to strategies for novel ways to use drugs in vivo.

All current approaches in biomedicine and research seek to control their behavior by rewiring-physically changing how the genes or proteins activate or suppress other genes or proteins (genomic editing, molecular biology). Here, the inventors took a different approach, treating these networks as computational agents and asked whether they could be *trained* into novel behaviors by experience—not physical rewiring but a history of stimuli. The inventors produced software that takes an existing GRN description and informs how to elicit the memory: what pattern of stimulation of which genes (nodes) will enable control of the important response node. The inventors' finding revolutionize many aspects of biomedicine and suggest that gene therapy can be avoided, and drug therapy can be used in specific pulsed regimes (not chronic exposures, as is done now) to address issues like the following: (i) why is there such variability in efficacy and side effects across patients? It could be because of the memories their GRNs have accumulated, and a knowledge of these can help predict who will respond how to a specific drug; (ii) why do drugs sometimes stop working, or become poorly tolerated over time? It could be because of habituation/sensitization respectively, and our software will enable this to be discovered and managed; and (iii) if an inert (harmless) drug is paired with a potent drug whose side effects prevent widespread use, associative learning (which our software can detect) can mean that after some paired exposures, the harmless drug alone might be enough to induce the effect (think Pavlov's dog and the bell). The inventors' software can be utilized to predict such memories in specific networks and suggest which nodes can serve as good targets for the inert drug.

By demonstrating that GRNs can form and store “memories” corresponding to their prior activity, the inventors' finding move the field closer to an understanding of developmental plasticity, as well as providing biomedical frameworks for understanding heterogeneity of response and side effects to drugs (due to GRN history) and to techniques to “train” GRNs for specific dynamics by patterned stimulation of key nodes (not requiring gene therapy or network rewiring). The examples of systems biology disclosed herein are quantitative and interdisciplinary, bridging molecular biology and cognitive science, and will be of significant interest in several fields.

### SUMMARY

Disclosed are methods for exploiting memory properties of biological systems such as gene regulatory networks (GRNs), protein networks, and biological pathways and to modulating the function of GRNs, protein networks, and biological pathways based on their memory properties. The disclosed methods may be utilized in order to treat diseases and disorders and in order to promote health.

The disclosed methods may be utilized in order to mitigate undesirable side effects of certain therapeutic agents. In the disclosed methods, a therapeutic agent having undesirable side effects may be administered to a subject with a paired inert agent in order to trigger a therapeutic response in the subject. Then subsequently, the previously paired inert agent can be administered alone without the therapeutic agent and the inert agent can trigger the therapeutic response without triggering the undesirable side effects.

The disclosed methods also may be utilized to predict a subject's response to a drug based on memory properties of biological systems triggered by the drug and modify a therapeutic regimen for the subject accordingly. The disclosed methods also may be utilized to predict whether a subject is likely to exhibit habituation or sensitization to a drug over time based on memory properties of biological systems triggered by the drug and modify a therapeutic regiment for the subject accordingly.

The disclosed methods may be utilized to mitigate side effects of a drug in a subject. The disclosed methods also may be utilized in order to break pharmacoresistance, habituation, or sensitization to a drug via administering the drug and/or a placebo under a specific dosage regimen. In some embodiments of the disclosed methods, a subject is administered a drug and/or a placebo under a pulsed regimen in contrast to a steady regimen.

The disclosed methods may exploit the natural learning abilities of biological systems to develop stimuli protocols and train the biological systems for better health responses.

The disclosed methods may target the memory setpoints of tissues and teach the tissue to be healthier. The disclosed methods may have a permanent therapeutic effect after the drugs administered in the methods are ceased to be administered.

### BRIEF DESCRIPTION OF THE FIGURES

FIG. 1. Extending associative learning paradigm to GRNs. (A-D) Pavlovian associative learning (collected from Wikimedia commons and modified). Whenever there is an unconditional stimulus or UCS (meat) there is a biological response R (salivation) but when there is a neutral stimulus or NS (bell), no response is observed. Now, if in an experiment, UCS and NS are applied together repeatedly (pairing of stimuli), the subject learns to associate the two stimuli and the NS becomes the conditioned stimulus, CS, which can activate R without the UCS. (E-H) our experiments in GRNs. Here, for associative learning in a GRN, nodes are considered as either a stimulus (marked in red if activated) or a response (marked in green if activated; black otherwise). If there is a path from each of the stimuli to R, R can learn their association.

FIG. 2. Definition of and functional relationship among the different memory types. The definition and abbreviations of the defined memory types are as follows. UCS Based Memory UM: R retains the activation by UCS after UCS deactivated. Pairing Memory (PM): R retains the repetitive activation by {UCS, NS} pair even after their deactivation. Transfer Memory (TM): activation by UCS alone (not pairing) converts NS to CS. Associative Memory (AM): paired activation of {UCS, NS}, converts NS to CS. Long Recall AM (LRAM): this conversion of NS to CS is permanent. Short Recall AM (SRAM): the conversion is temporary (the association is lost). Consolidation Memory (CM): the pairing of {UCS, NS} does not immediately turn NS into CS but eventually does so after an elapsed time. The overlap/hierarchy of the ovals represents the relationship between the different types and subtypes of memory.

FIGS. 3A and 3B. Flowchart of memory detection. The computational procedures for our evaluation of five kinds of memories are shown here, namely, UM, PM, TM, AM and CM. We consider each of the two types of AM, (LRAM and SRAM) as individual memory types. (A) Input of a GRN with a R-UCS pair and a probable list of NS. (B) The memory detection process. At the top of the figure we define the different modules frequently used in the section B. The process works as follows. 1) choose a stimulus set; 2) flip the state of the stimuli and fix them in that state, referred to as clamping; 3) simulate the BN for M time-steps; 4) record the state of R compared to its state prior to the clamping step; 5) unclamp the stimuli (allow them to update states), referred to as relaxation; 6) simulate the BN for M time-steps; 7) record the state of R compared to its state prior to relaxation; 8) choose a different stimulus set; 9) flip and clamp the stimuli; 10) simulate the BN for M time-steps; 11) record the state of R compared to its state prior to the clamping step 9; 12) relax the network; and 13) record the state of R. We deem a given stimulus-response combination as having elicited a specific type of memory if it satisfies a number of specific conditions described fully in the Methods.

FIG. 4. Minimal Random Boolean Networks (RBNs) have distinct memory types. Minimal BNs of the memory types (A) UM, (B) PM, (C) TM, (D) CM, and (E) AM. Each node of a network shows the Boolean equation to simulate the activation of the node. We present the symbols used in the equations in the legend.

FIG. 5. Associative Memory in biological GRNs. (A) Types of memory found in each of the 35 GRNs taken from the Cell Collective database. Associative memory was found in two of the GRNs: Aurora Kinase A in Neuroblastoma (B) and CD4+ T Cell Differentiation and Plasticity (B). For each network, we present an example of the stimuli-response combination where AM is obtained. (A) Cell Collective network where 3 genes, WEE1, PP2A, and TPX2 act as UCS, R and CS respectively. Activating TPX2 together with WEE1 enables TPX2 to activate PP2A, whereas previously only WEE1 did so. (C) Cell Collective network where IL4e, IL4, and GATA3 respectively act as UCS, R and CS. Activating GATA3 together with IL4e enables GATA3 to activate IL4, whereas previously only gene IL4e did so.

FIGS. 6A and 6B. Distribution of different memory types across diverse biological systems. The memory capacity of GRNs can be systematically classified according to their features. (A) A classification of GRNs based on whether they correspond to vertebrate or invertebrate species. This panel shows that vertebrate GRNs tend to contain more memory than the invertebrates, as quantified by the classification performance metrics: Accuracy=0.8, Sensitivity=0.94, Specificity=0.68, Positive predictive value=0.71, Negative predictive value=0.93 and AUC=0.81. (B) A classification of GRNs based on whether they correspond to generic cell types (not associated with particular cell types) or the differentiated (specific) cell types. This panel shows that the GRNs corresponding to the non-generic cell types tend to contain more memory than the generic ones, as quantified by the classification performance metrics: Accuracy=0.91, Sensitivity=0.94, Specificity=0.89, Positive predictive value=0.88, Negative predictive value=0.94 and AUC=0.92. Classification was performed as follows. First, the memory capacity of each GRN was computed as the proportion of memory within the total that included the ‘no-memory’ type. Then, if the memory capacity of a GRN exceeded 50% it was categorized under the ‘memory’ class, or in the ‘no memory’ class otherwise. The standard binary classification metrics reported above were computed based on the associated confusion matrix containing the number of True positives (TP), False positives (FP), True Negatives (TN) and False Negatives (FN) where the ‘memory’ class is the ‘positive’ class, and the ‘no-memory’ class is the ‘negative’ class. As per standard definitions, Accuracy is the proportion of TP and TN among the total number of instanced; Sensitivity is the proportion of TP among the actual positive instances; Specificity is the proportion of TN among the actual negative instances; Positive predictive value is the proportion of TP among the predicted positive instances; Negative predictive value is the proportion of TN among the predicted negative instances; AUC is the area under the ROC curve, which can be interpreted as the probability that the classifier will rank a randomly chosen positive instance higher than a randomly chosen negative instance.

FIG. 7. Distribution of Memory in Different Sizes of Random Boolean Networks (RBNs). Pie charts (A-C) show the memory distributions in RBNs with 5, 15 and 25 nodes (100 RBNs for each case). (D) shows the comparative distribution of different memories in various sizes (5, 10, 15, 20 and 25) of RBNs. The pie charts (E-G) shows the memory vs. no memory distribution in GRNs. (H) shows the distribution of different memory types across biological GRNs of increasing size.

FIG. 8A-8C. Biological GRNs exhibit unique memory properties. (A) violin plots of the set of GRNs from the Cell Collective database (*) compared (in terms of memories) to their configuration models. We show the mean (black line), median (red line), 5^{th} percentile (teal line) and 95^{th} percentile (pink line). The actual frequency of memory of the real GRN is represented as a red star. Only the “Aurora Kinase in Neuroblastoma” network from Cell Collective is plotted. The violin plots of memories for all the 35 GRNs are given in supplementary material (Supplement 3, plots 1-35). We calculated the conditional entropy among the different types of memories of GRNs and Configuration models, normalized these conditional entropies, applied Gaussian smoothing and visualized the results obtained from (B) GRNs and (C) configuration models. Notably, GRNs are distinct compared to their randomized counterparts in the context of predicting the availability of a certain type of memory given the appearance of any other type of memory.

FIG. 9. The axis of persuadability is a multidimensional continuum on which any system can be placed, with respect to what kind of strategy is optimal for prediction and control. On the far left are the simplest physical systems, e.g. a mechanical clock. These cannot be persuaded, argued with, or even rewarded/punished-only physical hardware-level “rewiring” is possible if one wants to change their behavior. On the far right are human beings whose behavior can be radically changed by a communication that encodes a rational argument that changes the motivation, planning, values and commitment of the agent receiving this. This continuum is the framework for our hypothesis that a genetic network can learn.

FIG. 10. Biological networks: gene-regulatory networks (GRN), protein networks, for example carcinoma protein network, and metabolic networks consist of nodes connected by functional relationships (e.g. activation/repression) in some sort of topology.

FIG. 11. We designed, built, and deployed the first automated training and testing device for planaria and tadpoles [98], which we used to study memory during brain regeneration [99] and the plasticity of vision in animals with eyes in aberrant locations [24, 25, 100].

FIG. 12. Extending associative learning paradigm to GRNs. The sequence of behavioral changes is driven by particular combinations of stimuli in every phase of associative memory. The stimuli-response mapping is shown for each phase, and the relevant ones are marked with a green box. For example, during the pre-association phase (A), the relevant combinations are where either the individual stimuli or no stimuli are presented. (B) During the association phase, both stimuli are presented at the same time. The most important observation to be made here is the distinction between the stimuli-response mappings of the pre-association and the post-association phases (C). In particular, the salivation response to CS during post-association is altered compared with that in the pre-association phase. This is accomplished by the activation of memory during the association phase. In other words, the dog with a memory of the association between UCS and CS responds to the latter stimulus differently. This altered behavior is a result of memory, as shown by the equation at the bottom of (C). The underlying Boolean network model shows the rules of behavior of the memory (M) and the response (R) nodes. The phenomenon of associative memory can also be understood in symbolic terms as follows. During the pre-association phase M is not activated as per the relevant stimuli-response combinations. Thus, if we set M=OFF in the rule for R, we get a rule that says that R can be triggered by UCS only (R←UCS). During the association phase, the joint presentation of the stimuli activates M. Finally, during the post-association phase, if we set M=ON in the rule for R, we get a rule that says R can be triggered by either UCS or CS in a symmetrical way (R←UCS OR CS). In other words, association casts UCS and CS as equivalent from the point of view of R.

FIG. 13. Time series data of a GRN's evaluation for associative memory. This trace describes the run time state changes in evaluating associative memory of a mammalian cell cycle network. (A) In the mammalian cell cycle network 2006, the genes used as UCS, NS/CS, and R are highlighted with blue, red, and cyan respectively. With these respective colors the states of UCS, NS/CS, and R in different plots are defined. A downward arrow in each plot shows the start of the activation of the corresponding stimuli. In each panel, we show the 10 past states of a stimulus to depict its state change upon the activation at time 0. (B) The resultant states of R, observed from activation of UCS and NS, respectively, before training: R gets activated with onset of UCS but NS cannot trigger R. (C) Pairing (training) experiment shows the successful activation of R. (D) After training, activation of the previously neutral stimulus causes R to be activated, confirming that the experience of paired stimuli has converted the NS node to a CS. (E) As further confirmation of stable causality established between CS and R by training, we first deactivate CS, to see if R gets deactivated, and then reactivate the CS to ensure that it can activate R again.

FIG. 14. Mapping between various tools and the most related cognitive concepts. A taxonomy mind-map of tools to analyze cognitive phenomena, broadly decomposed into deterministic and statistical. The deterministic toolset further consists of dynamical and algorithmic sub-categories, while the statistical set consists of the information-theoretic and least-action principles sub-categories.

FIG. 15. A single chamber, the control loop for a single Session, and the control loop for a whole experiment

FIG. 16. A minimal sample network of how associative memory is tested by our algorithm.

FIG. 17. An example of the type of data expected to be generated in section (3). Shown here coming from an analysis of ODE (continuous) models for breaking pharmacoresistance (labeled as memory) where each “attempt” is a stimulus that has been predicted to abolish the memory and showing predicted successful and unsuccessful cases (the network represented by yellow bars in the 2^{nd} row, and to some extent the network shown in the first row as blue bars).

FIG. 18. A cognitive view of associative learning as offered by the tools of dynamical systems. Each panel illustrates the flow together with the phase portrait of the GRN in the space of p and w2 (the w1 axis is ignored for conciseness, since it is not informative). Here, ‘response’ represents the concentration levels of p. The red and green curves in the top and bottom panels, respectively, depict representative trajectories. The red and green trajectories are each split over time across the horizontal panels in their respective rows, as depicted by grey dashed lines connecting the consecutive pieces whose endpoints are marked by colour filled circles. Note that the endpoint of one piece and the starting point of the following piece are of the same colour since they represent the same states. The overall initial state of the two trajectories (green filled circle) are the same. Also shown in each panel are the stable equilibrium and saddle points. The top panels show CS alone cannot evoke a response (red trajectory eventually reaches a low-response state in panel (c)). The bottom panels show that following an association of CS with US, CS alone can evoke a response (the green trajectory eventually reaches a high-response state in panel (f)). Notice that there are two attractors (hence two basins of attraction) when CS alone is applied (right panels). In the dynamical systems view, associative learning is about steering the internal state associated with CS (w2) into the basin of attraction associated with high value of p with the help of application of US. More specifically, a minimum value of w2 is necessary and sufficient to evoke a high response; this is termed the ‘learning threshold’ (the black dashed line in panels (a,c,f)). Here, associative learning is accomplished by w1 ‘shepherding’ w2 above the learning threshold.

FIG. 19. Associative memory (AM) for nicotine response. A human ovarian cancer cell line (HeLa) was cultured in DMEM cell culture media in a cell culture flow cell. The cells were treated with nicotine by introducing a DMEM/nicotine solution through the microfluidics of the cell culture device for a brief period, followed by continued flow of DMEM alone for a period of time before the flow of DMEM was stopped and the cells were left to rest in cell culture media. Ca⁺² flux was quantitatively measured using a standard fluorescence method. Pulsing with nicotine caused a decrease in fluorescence that continued to decline even after the flow of media was stopped. Upon retreatment of the same cell culture with fresh nicotine and DMEM, the decrease in fluorescence was replicated. Over 9 repetitions of nicotine pulsing, followed by DMEM alone and resting (for a total of 10 treatments in total), the decrease in fluorescence was nearly identical. After the 10th treatment with nicotine, the cells were pulsed with DMEM alone (without nicotine), and the decrease in fluorescence was observed again. After stopping the fluid flow, it was restarted-again without any nicotine present- and the decrease in fluorescence was replicated again. This response to fluid flow in the absence of nicotine was replicated 4 times. HeLa cells do not typically have this response simply by being exposed to fluid flow. In this experiment, associative memory was demonstrated by training fluid flow to replicate the biological response to nicotine. In this example, the UCS is nicotine, and the NS is fluid flow, which is converted to a CS after nicotine training based on the principle of associative memory.

### DETAILED DESCRIPTION

The following discussion is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the disclosure. Thus, embodiments of the disclosure are not intended to be limited to embodiments shown, but are to be accorded the widest scope consistent with the principles and features disclosed herein. The following detailed description is to be read with reference to the figures. The figures, which are not necessarily to scale, depict selected embodiments and are not intended to limit the scope of embodiments of the disclosure. Skilled artisans will recognize the examples provided herein have many useful alternatives and fall within the scope of embodiments of the disclosure.

### Definitions and Terminology

Disclosed are methods for exploiting memory properties of biological systems such as gene regulatory networks (GNRs). The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a therapeutic agent” and “a stimulus” should be interpreted to mean “one or more therapeutic agents” and “one or more stimuli,” respectively. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Methods for Modulating the Function of Biological Regulatory Networks in Health and Disease by Exploiting their Memory Properties

The disclosed subject matter relates to memory properties of biological systems such as gene regulatory networks (GNRs). In particular, the disclosed subject matter relates to methods and systems that may be utilized in order to treat diseases and disorders and in order to promote health.

In some embodiments, the disclosed methods relate to methods for treating a disease or disorder characterized by a biological system such as a gene regulatory network (GRN) in a subject in need thereof. The disease or disorder also is characterized by a therapeutic agent that triggers a response in the GRN and a corresponding therapeutic response to the disease or disorder in the subject when the therapeutic agent is administered to the subject. Optionally, the therapeutic agent may exhibit undesirable side effects when the therapeutic agent is administered to the subject, for example, at a standard therapeutic dose.

The disclosed treatment methods may include: (i) administering to a subject in need thereof a therapeutic agent and an inert agent, where the therapeutic agent triggers a response in the GRN and a corresponding therapeutic response in the subject when the therapeutic agent is administered to the subject without the inert agent, and where the inert agent does not trigger a response in the GRN and a corresponding therapeutic response in the subject when the inert agent is administered to the subject without the therapeutic agent; and (ii) subsequently repeating step (i) one or more times until the inert agent triggers a response in the GRN and a corresponding therapeutic response in the subject when the inert agent is administered to the subject without the therapeutic agent, thereby treating the disease or disorder characterized by the GRN in the subject by administering the inert agent. The disclosed treatment methods therefore may comprise a step of converting the inert agent into an agent that exhibits at least a temporal therapeutic effect. The disclosed methods further may comprise a step of (iii) administering the inert agent to the subject until the inert agent ceases to trigger a response in the GRN and a therapeutic response in the subject, for example, where the inert agent exhibits a temporal therapeutic effect that ceases after repeated administration to the subject (e.g., after the inert agent is administered to the subject 2, 3, 4, 5, 6, 7, 8, 9, or 10 times).

In the disclosed treatment methods, the disease or disorder to be treated is associated with a GRN and a therapeutic agent that triggers a response in the GRN. In some embodiments, the GRN is selected from the group consisting of “Aurora Kinase A in Neuroblastoma,” “CD4+ T Cell Differentiation and Plasticity,” “Human Gonadal Sex Determination,” “B cell differentiation,” and “Fanconi Anemia and Checkpoint Recovery.” In some embodiments of the disclosed treatment methods, suitable diseases or disorders may include but are not limited to cancer, metabolic diseases or disorders (e.g., diabetes), and developmental diseases or disorders.

In some embodiments, the disclosed treatment methods may be performed in order to reduce the effective therapeutic dose of a therapeutic agent in a subject in need thereof, for example, where a standard dose of a therapeutic agent results in undesirable side effects when the standard dose is administered to the subject. The methods may include: (i) administering to the subject the standard dose of a therapeutic agent and a dose of an inert agent, wherein the standard dose of the therapeutic agent triggers a response in the GRN and a corresponding therapeutic response in the subject (and corresponding side effects in the subject) when the standard dose of the therapeutic agent is administered to the subject without the dose of the inert agent, and wherein the dose of the inert agent does not trigger a response in the GRN and a corresponding therapeutic response in the subject (and corresponding side effects in the subject) when the dose of the inert agent is administered without the standard dose of the therapeutic agent; and (ii) subsequently repeating step (i) one or more times until the dose of the inert agent triggers a response in the GRN and a corresponding therapeutic response in the subject without corresponding side effects when the dose of the inert agent is administered to the subject without the standard dose of the therapeutic agent, thereby treating the disease or disorder characterized by the GRN in the subject. The disclosed treatment methods may include step (iii) administering to the subject a lower dose of the therapeutic agent and optionally a dose of the inert agent, wherein the subsequently administered lower dose of the therapeutic agent is lower than the previously administered stand dose of the therapeutic agent in step (i) and the lower dose of the therapeutic agent triggers a response in the GRN and a therapeutic response in the subject but the lower dose of the therapeutic agent does not trigger undesirable side effects in the subject (or the lower dose of the therapeutic agent only triggers reduced side effects in the subject), thereby treating the disease or disorder while mitigating the undesirable side effects in the subject. Optionally, the methods further may comprise step (iv) continuing to administer the lower dose of the therapeutic agent to the subject until the lower dose of the therapeutic agent ceases to trigger a response in the GRN and a corresponding therapeutic response in the subject.

The disclosed subject matter also includes systems and methods for determining whether a determining whether a gene regulatory network (GRN) exhibits memory, and components of the systems and methods.

In some embodiments, the disclosed systems comprise at least one hardware processor that is programmed to perform one or more of the following steps: (A) simulating administering to the GRN an unconditioned stimuli (UCS) and determining whether the UCS triggers a response by the GRN; and (i) if the UCS does not trigger a response by the GRN, then repeating step (A) using another different UCS until the UCS triggers a response by the GRN; or (ii) if/when the UCS triggers a response by the GRN, then allowing the GRN to relax and simulating administering the UCS to the GRN and determining whether the GRN exhibits UCS-based memory (UM), and if the GRN does not exhibit UM then proceeding to step (B) or if the GRN exhibits UM then optionally completing the method; (B) simulating administering to the GRN a combination of an unconditioned stimulus (UCS) and a neutral stimulus (NS) and determining whether the combination of the UCS and the NS triggers a response by the GRN; and (i) if the combination of the UCS and the NS does not trigger a response by the GRN, then repeating step (B) using a combination of the UCS and another different NS until the combination of the UCS and the NS triggers a response by the GRN; or (ii) if/when the combination of the UCS and the NS triggers a response by the GRN, then allowing the GRN to relax and simulating administering the combination of the UCS and the NS and determining whether the GRN exhibits pairing memory (PM), and if the GRN does not exhibit PM then proceeding to step (C) or step (D) and if the GRN exhibits PM then optionally completing the method; (C) simulating administering to the GRN an unconditioned stimuli (UCS) and determining whether the UCS triggers a response by the GRN; and (i) if the UCS does not trigger a response by the GRN, then repeating step (C) using another different UCS until the UCS triggers a response by the GRN; and (ii) if/when the UCS triggers a response by the GRN, then allowing the GRN to relax and simulating administering a NS to the GRN and determining whether the GRN exhibits transfer memory (TM), and if the GRN does not exhibit TM then proceeding to step (D) or if the GRN exhibits TM then optionally completing the method; (D) simulating administering to the GRN a combination of an unconditioned stimulus (UCS) and a neutral stimulus (NS) and determining whether the combination of the UCS and the NS triggers a response by the GRN; and (i) if the combination of the UCS and the NS does not trigger a response by the GRN, then repeating step (D) using a different combination of another different UCS and/or another different NS until the combination of the UCS and the NS triggers a response by the GRN; or (ii) if/when the combination of the UCS and the NS triggers a response by the GRN, then allowing the GRN to relax and simulating administering the NS and determining whether the GRN exhibits associative memory (AM), and if the GRN does not exhibit AM then proceeding to step (E), or if the GRN does exhibit AM then optionally completing the method or optionally proceeding to step (D) (iii); or (iii) if the GRN exhibits AM, then allowing the GRN to relax and simulating administering the NS and determining whether the GRN exhibits long recall AM (LRAM), and if the GRN does not exhibit LRAM, then determining that the GRN exhibits short recall AM (SRAM) and optionally repeating step (D) using a different combination of another different UCS and/or another different NS until the GRN exhibits LRAM; and (E) after performing step (D), allowing the GRN to relax and simulating administering the NS and determining whether the GRN exhibits consolidation memory (CM), and if the GRN exhibits CM optionally completing the method or if the GRN does not exhibit CM then determining that the GRN does not exhibit memory. The disclosed systems further may comprise software for programming the hardware processor to perform one or more of steps (A), (B), (C), (D), and (E).

The disclosed methods for determining whether a gene regulatory network (GRN) exhibits memory may comprise one or more of the following steps: (A) administering (or simulating administering) to the GRN an unconditioned stimuli (UCS) and determining whether the UCS triggers a response by the GRN; and (i) if the UCS does not trigger a response by the GRN, then repeating step (A) using another different UCS until the UCS triggers a response by the GRN; or (ii) if/when the UCS triggers a response by the GRN, then allowing the GRN to relax and administering (or simulating administering) the UCS to the GRN and determining whether the GRN exhibits UCS-based memory (UM), and if the GRN does not exhibit UM then proceeding to step (B) or if the GRN exhibits UM then optionally completing the method; (B) administering (or simulating administering) to the GRN a combination of an unconditioned stimulus (UCS) and a neutral stimulus (NS) and determining whether the combination of the UCS and the NS triggers a response by the GRN; and (i) if the combination of the UCS and the NS does not trigger a response by the GRN, then repeating step (B) using a combination of the UCS and another different NS until the combination of the UCS and the NS triggers a response by the GRN; or (ii) if/when the combination of the UCS and the NS triggers a response by the GRN, then allowing the GRN to relax and administering (or simulating administering) the combination of the UCS and the NS and determining whether the GRN exhibits pairing memory (PM), and if the GRN does not exhibit PM then proceeding to step (C) or step (D) and if the GRN exhibits PM then optionally completing the method; (C) administering (or simulating administering) to the GRN an unconditioned stimuli (UCS) and determining whether the UCS triggers a response by the GRN; and (i) if the UCS does not trigger a response by the GRN, then repeating step (C) using another different UCS until the UCS triggers a response by the GRN; and (ii) if/when the UCS triggers a response by the GRN, then allowing the GRN to relax and administering (or simulating administering) a NS to the GRN and determining whether the GRN exhibits transfer memory (TM), and if the GRN does not exhibit TM then proceeding to step (D) or if the GRN exhibits TM then optionally completing the method; (D) administering (or simulating administering) to the GRN a combination of an unconditioned stimulus (UCS) and a neutral stimulus (NS) and determining whether the combination of the UCS and the NS triggers a response by the GRN; and (i) if the combination of the UCS and the NS does not trigger a response by the GRN, then repeating step (D) using a different combination of another different UCS and/or another different NS until the combination of the UCS and the NS triggers a response by the GRN; or (ii) if/when the combination of the UCS and the NS triggers a response by the GRN, then allowing the GRN to relax and administering (or simulating administering) the NS and determining whether the GRN exhibits associative memory (AM), and if the GRN does not exhibit AM then proceeding to step (E), or if the GRN does exhibit AM then optionally completing the method or optionally proceeding to step (D) (iii); or (iii) if the GRN exhibits AM, then allowing the GRN to relax and administering (or simulating administering) the NS and determining whether the GRN exhibits long recall AM (LRAM), and if the GRN does not exhibit LRAM, then determining that the GRN exhibits short recall AM (SRAM) and optionally repeating step (D) using a different combination of another different UCS and/or another different NS until the GRN exhibits LRAM; and (E) after performing step (D), allowing the GRN to relax and administering (or simulating administering) the NS and determining whether the GRN exhibits consolidation memory (CM), and if the GRN exhibits CM optionally completing the method or if the GRN does not exhibit CM then determining that the GRN does not exhibit memory.

### EXAMPLES

The following examples are illustrative and should not be interpreted to limit the scope of the claimed subject matter.

### Example 1

### Title—Gene Regulatory Networks Exhibit Several Kinds of Memory: Quantification of Learning in Biological and Random Transcriptional Networks

Reference is made to the manuscript Biswas et al., “Gene Regulatory Networks Exhibit Several Kinds of Memory: Quantification of Learning in Biological and Random Transcription Networks,” iScience 2021 Mar. 19; 24 (3): 102131, published online 2021 Feb. 1. doi: 10.1016/j.isci.2021.102131, the content of which is incorporated herein by reference in its entirety.

Transcriptional networks are a fundamental regulatory mechanism in biology, enabling rich computational dynamics that link the levels of input genes to those of effectors (responses) in embryogenesis and adult physiology. Understanding how gene regulatory networks (GRNs) process information is thus of major interest for evolutionary developmental biology as well as biomedicine. An important knowledge gap concerns the ways in which GRN dynamics and responses change over time. Because GRNs guide both morphogenesis and transitions between health and disease states, it is critical to understand how long-term changes in network properties could arise, and how diverse subsequent GRN behaviors could be induced by specific histories of stimuli (transcriptional inputs). Hypothesizing that such networks could exhibit memory, we created a computational framework for defining and identifying diverse types of memory in candidate GRNs. We show that biological GRNs from a wide range of model systems are predicted to possess different types of memory, depending on the composition and timing of stimuli/response dynamics, and the extent to which they stably persist after transient input events. We show that the probability of finding a specific type of memory in a biological GRN is predictive of finding others. Crucially, some GRNs show the capacity for associative learning via classical conditioning. The ability of a network to change its structure as a function of signaling history, enabling new outcomes to be triggered by previously neutral stimuli, offers a new strategy for the biomedical use of powerful drugs with undesirable side effects, and for understanding the variability and time-dependent changes of drug action. We found evidence of natural selection favoring GRN memory, as observed from a comparison of the memory profiles of biological GRN models with associated randomized ensembles. Vertebrate GRNs overall have a stronger capacity for memory compared to invertebrate GRNs; moreover, the capacity for memory is most prevalent in differentiated metazoan cells. Taken together our data reveal a novel computational aspect of GRN function and suggest a control policy for networks focusing on regimes of experience, not transcriptional rewiring. This strategy may have significant implications for biomedical efforts to control complex in vivo dynamics without genomic editing or transgenes.

### Introduction

Gene regulatory networks (GRNs) are key drivers of embryogenesis, and their importance persists through all stages of life (1, 2). Understanding the dynamics of GRNs is thus of high priority not only for the study of evolutionary developmental biology (3, 4), but also for the prediction and management of disease states (5-7). Much work has gone into computational inference of GRN models (8, 9), and the development of algorithms for predicting their dynamics over time (10). However, the field has been largely focused on rewiring-modifying the inductive and repressive relationships between genes—to control outcome. This is often impractical in biomedical contexts, and even in amenable model systems it is often unclear what aspects of the network should be altered to result in desired system-level behavior of the network. Dynamical systems approaches have made great strides in understanding how GRNs settle on specific stable states (11, 12). However, there are significant knowledge gaps concerning temporal changes in GRN dynamics, their plasticity, and the ways in which they could be rationally controlled.

Thus, an important challenge in developmental biology and biomedicine is the identification of novel methods to control GRN dynamics without having to solve the difficult inverse problem (13) of inferring how to reach desired system-level states by manipulating individual node relationships, and without transgenes or genomic editing. A view of GRNs as a computational system, which converts activation levels of certain genes (inputs) to those of effector genes (outputs), suggests an alternative strategy: to control network behavior via inputs-spatiotemporally regulated patterns of stimuli. This approach is motivated by the advances of neuroscience, in which nervous systems and artificial neural networks learn from experience. Recent advances in the field of basal cognition (memory and learning in aneural and pre-neural organisms (14)) have revealed a broad class of systems, from molecular networks (15) to physiological networks in somatic organs (16, 17), that exhibit plasticity and history-based remodeling. Could GRNs likewise exhibit history-dependent behavior that could help understand variability and be exploited to control their function by modulating the temporal sequence of inputs?

Based on the remarkable flexibility observed at the anatomical and physiological levels (18-23), and the conceptual similarity between GRNs and neural networks (24-26), we hypothesized that GRNs may have the property of memory: altering their response to future events based on a specific history of stimuli. We hypothesized that this takes place via changes at the level of the dynamical system state space, not requiring changes in transcriptional relationships between genes, and that it would be a general property enriched in biological GRNs.

If true, this kind of GRN plasticity would have major implications along two lines. First, it would suggest novel developmental programs where dynamic gene expression could result from GRNs whose functional behavior was shaped by prior biochemical interactions and not genomically hardwired. Second, it would suggest a novel approach to biomedical interventions complementing gene therapy: drug strategies with temporally controlled delivery regimes could be designed to train GRNs to produce specific outcomes or to prevent disease states. Moreover, an understanding of GRN historicity could help explain the wide divergence of efficacy and side effects to drugs across patients and across even clonal model systems (27).

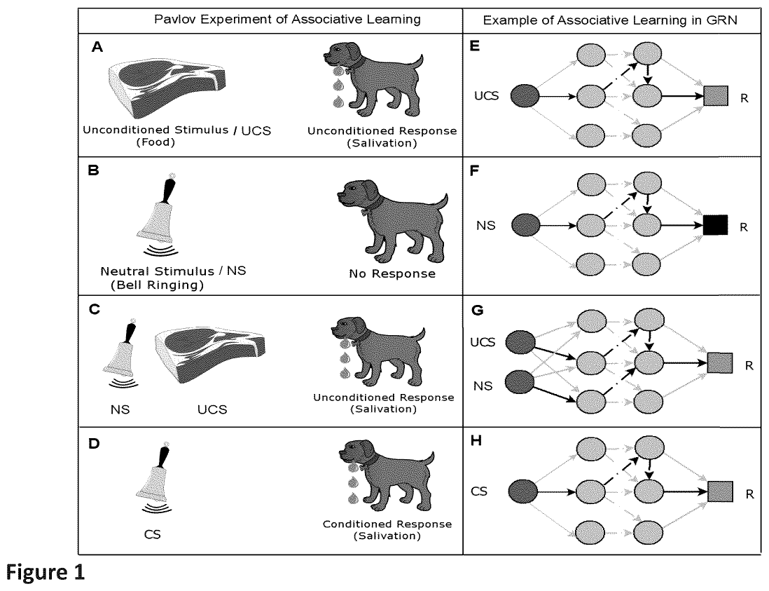

One especially intriguing possibility concerns associative learning (28, 29). The textbook experiment by Pavlov illustrates associative learning in a specific form known as “classical conditioning” (30, 31) (FIG. 1A-D). Here, initially, the dog naturally salivates when it smells food (FIG. 1A), termed the unconditioned stimulus (UCS). The dog does not salivate when it hears a bell ring (FIG. 1B), making the bell the neutral stimulus (NS). The smell of food and the sound of a bell are unrelated stimuli, and only one, the UCS, induces the dog's salivation (the response R). In this experiment, the dog is exposed to the UCS and NS at the same time repeatedly (FIG. 1C). Gradually, the dog learns to associate the NS with the UCS, to the point where it responds to the bell alone as if food is present, transforming the NS to the Conditioned Stimulus (CS) by producing the response R (FIG. 1D). Although associative learning is traditionally studied as a neural phenomenon, many different types of dynamical systems could instantiate it (14, 32-35) (FIG. 1E-H). Indeed, the original experiments of Pavlov showed associative and other kinds of learning within organ systems (36, 37), in addition to the well-known learning of the animal via its brain.

In biomedical contexts, drugs targeting specific network nodes are highly effective in laboratory studies but too toxic to use long-term in patients (38). We reasoned that if associative memory existed in GRNs, predictive algorithms could be developed to reveal which stimuli can be used to trigger responses via a paired training paradigm. In this case, the network would associate the effects (R) of a powerful but toxic drug (UCS) with a harmless one (NS, which would become the CS). It might then be possible (for at least some time) to treat the patient with the neutral drug (NS) to obtain the desired therapeutic response of the UCS without the side effects (FIG. 1E-H).

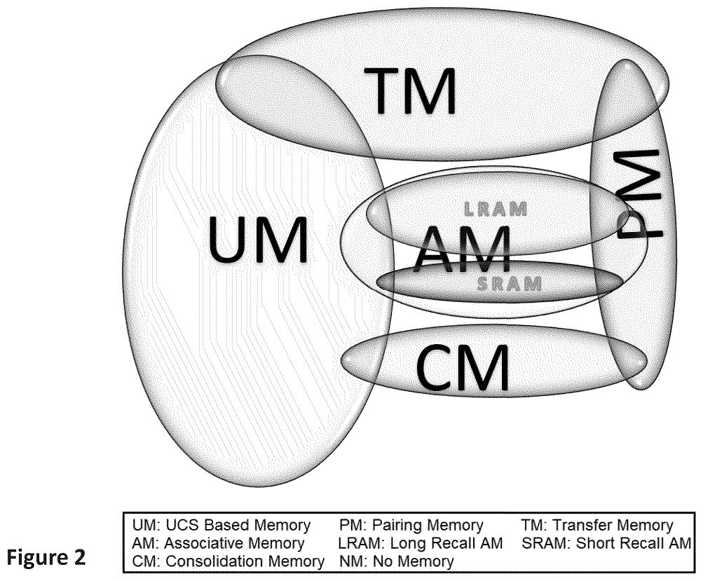

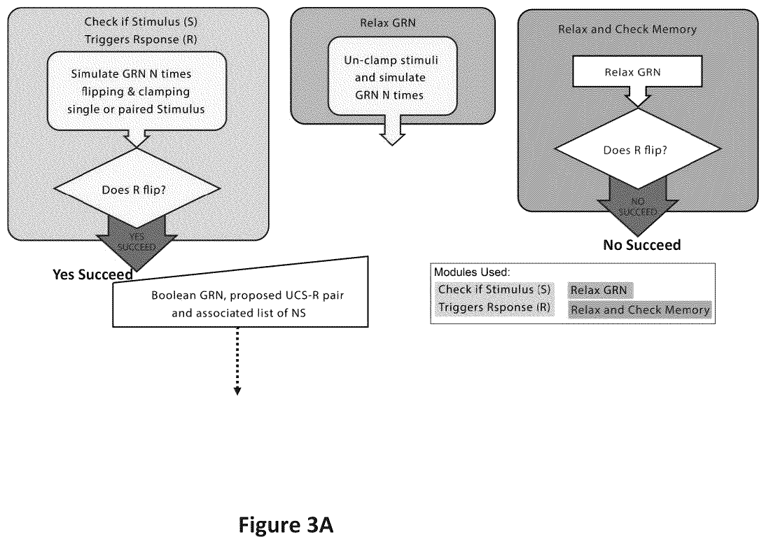

The presence of a kind of learning in GRNs has been suggested in specific cases (11, 25, 39, 40), but there has been no systematic study of memory across diverse GRNs or analysis of possible different kinds of memories that may exist and the relationships between them. It is also unknown whether memory is a property of all networks (e.g., random ones) or whether biological GRNs exhibit unique memory types. Here, we comparatively analyze the definitions of memory in the context of animal behavior and GRNs, providing a taxonomy of learning types appropriate for GRNs and other networks like protein pathways. We rigorously define the kinds of memory that could be present in GRNs (FIG. 2), and then produce an algorithm to systematically test any given GRN for the presence of different types of memory with different choices of network nodes as stimuli targets. Analyzing a database of known GRNs (Supplement 1, Table 1) from a wide range of biological taxa, we show that surprisingly, several kinds of memory can be found, including associative memory. We develop configuration models (randomized versions of each biological GRN) to demonstrate that the amount of memory found in a GRN is not governed solely by node number and edge density and that real biological GRNs are distinct in their types and degrees of memory compared to similar random networks. Comparing GRN data with analysis of configuration models revealed that true biological networks have disproportionately more memory (suggesting that evolution may favor networks with memory properties). We also identified statistical relationships between the likelihood of a given network exhibiting a particular kind of memory and two factors: what other memory types it may have, and what kind of cell/organism the GRN is from. Taken together, our results provide a novel way to understand and control GRN behavior, establishing a software framework for discovery of memory and thus actionable intervention strategies for biomedical, developmental, and synthetic biology settings.

### Results

Transcriptional networks can exhibit multiple kinds of memory. A GRN is a model of transcriptional control consisting of genes and their mutual regulations (8, 41). Each gene has a basal expression level that applies when the gene is neither regulated by any external stimuli nor influenced by other genes (through their encoded proteins). Basal expression levels change when a gene is activated via regulation, which then in turn may modulate others (42, 43).

We formally define “memory” in this context as a phenomenon describing the relationship between two sets of genes, namely “stimulus” and “response” that satisfies the following conditions: (i) the stimulus activates the response; and (ii) the response retains its activation state even after deactivation of the stimulus (the existence of history). The fundamental signature of memory is its temporality—a long-lasting and stimulus-specific change induced by a transient experience. We consider individual nodes of a Boolean GRN as the potential targets of external stimuli, and able to produce a response (output or effector nodes). For example, a specific transcript can be upregulated by some exogenous factor triggering its expression, and the appearance of a given gene product (e.g., secretion of an important hormone or growth factor) can be considered the circuit's response. For applications, we are especially interested in nodes which can be readily stimulated with small molecule drugs, and for response, we are interested in nodes that control key drivers of health and disease (e.g., the levels of calcium, pH, immune activation, cell differentiation, etc.). The challenge then, for any given network and response of interest, is to computationally identify the correct nodes that may serve as inputs, and a temporal stimulation regime for those stimulus node(s), that will result in desired changes in response activity over time.

Specifically, we consider two types of stimulus nodes, namely unconditional stimulus (UCS) and neutral stimulus (NS), and a single response node (R). The first type of stimulus, UCS, is capable of triggering R, and the second type, NS, is initially neutral to R but may be conditioned such that it now becomes a driver to flip R. In classical conditioning of a GRN, we pair the NS with the UCS and apply both repeatedly so that R can learn the association between the two stimuli. Later, we test in the absence of the UCS to see if R is driven by the NS alone (if true, NS can now be called a Conditioned Stimulus CS). The taxonomy of possible memory types in such systems, and their relationships, are schematized in FIG. 2, including: Associative Memory, UCS Based Memory, Pairing Memory, Transfer Memory, Associative Memory (including two of its sub-categories: Long Recall Associative Memory and Short Recall Associative Memory), and Consolidation Memory.

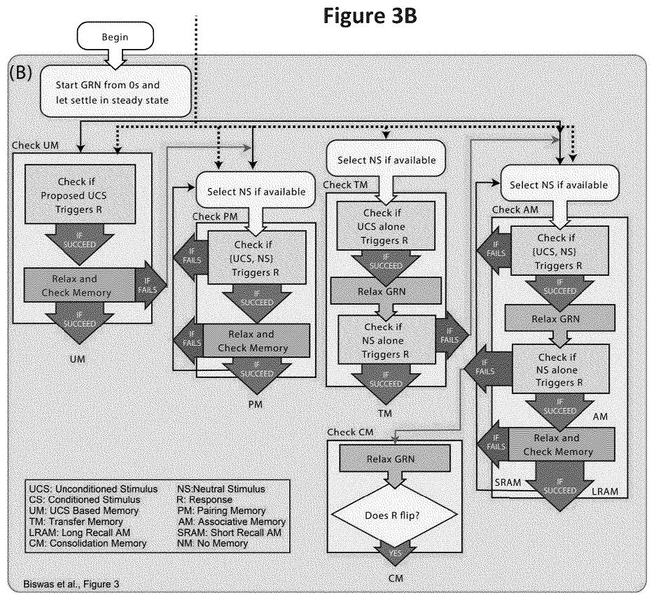

We tested (using the paradigm shown in FIG. 3) many models of a diverse range of biological systems obtained from the publicly available dataset Cell Collective (44). To measure the uniqueness of the memory profiles of real biological GRN models, we computed the memory profiles of thousands of Configuration Models created via a randomization of each of the biological GRNs; we also tested hundreds of Random Boolean Networks (RBNs) to study the prevalence and type of memory properties of networks in general.

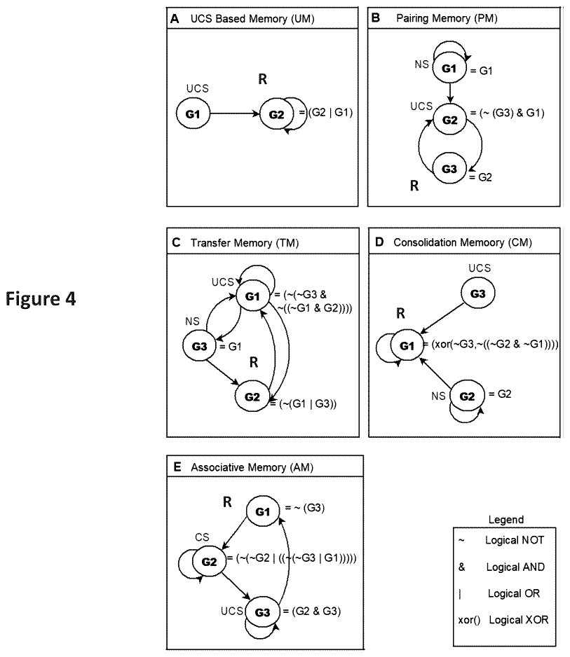

We first sought to discover minimal networks showing each kind of memory, to serve as prototypical examples and also to guide design of novel GRNs for synthetic biology applications that exploit transcriptional memory. At minimum a network needs 2 nodes (UCS and R) to form UM and 3 nodes (UCS, R and NS) to form any other type of memories. To test the topographies and motifs associated with each type of memory, we created thousands (10000 for each case) of RBNs and evaluated each memory using our toolkit (see Methods). The smallest networks discovered to be sufficient for each type of memory are shown in FIG. 4. We conclude that even fairly simple networks, readily accessible to synthetic biology construction, can give rise to memory functionality.

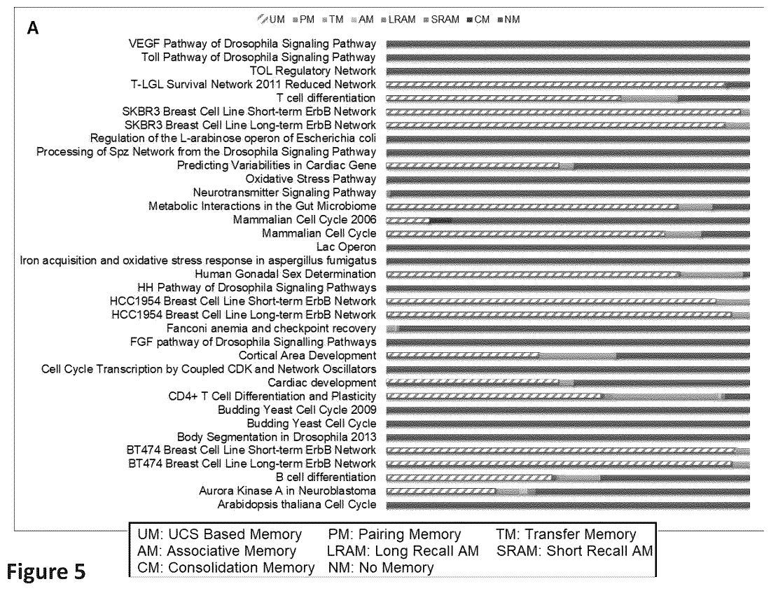

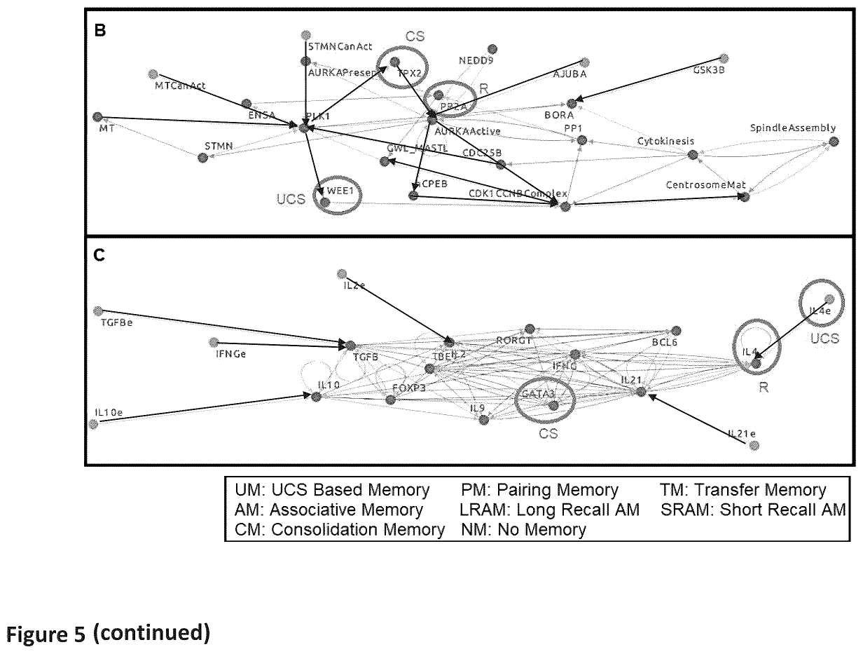

Biological GRNs possess various memory types: an analysis across taxa. We next tested 35 biological GRNs (all GRNs<25 nodes in size, from Cell Collective (44) for each kind of memory (FIG. 5A). These included GRNs at different strata of evolution (prokaryotes and eukaryotes), cancer, diverse metabolic processes in adult and embryonic stages of mammals, cellular signaling pathways in invertebrate and plants, etc. For each network, the prevalence of each type of memory was analyzed by assessing the number of different combinations of nodes that can serve as UCS-R-NS.

Three (3) out of 35 (8.57%) GRNs exhibited no feasible stimuli-response (UCS-R-NS) combinations with memory. For those GRNs with memories (rest 32 in 35), UM is the most prevalent type of memory, followed by TM. AM and PM memory types are somewhat rarer (only 5 out of 35 GRNs). AM appeared in “Aurora Kinase A in Neuroblastoma”, “CD4+ T Cell Differentiation and Plasticity”, “Human Gonadal Sex Determination”, “B cell differentiation”, and “Fanconi Anemia and Checkpoint Recovery” GRNs, among which the first 2 GRNs (highlighting a certain combination of stimuli-response for each) are shown in FIGS. 5B and C respectively. In each of the first 4 GRNs, AM and PM occurred together. For each GRN, the percentage of combinations where a certain memory appeared out of all feasible combinations are listed in Supplement 1, Table 2.

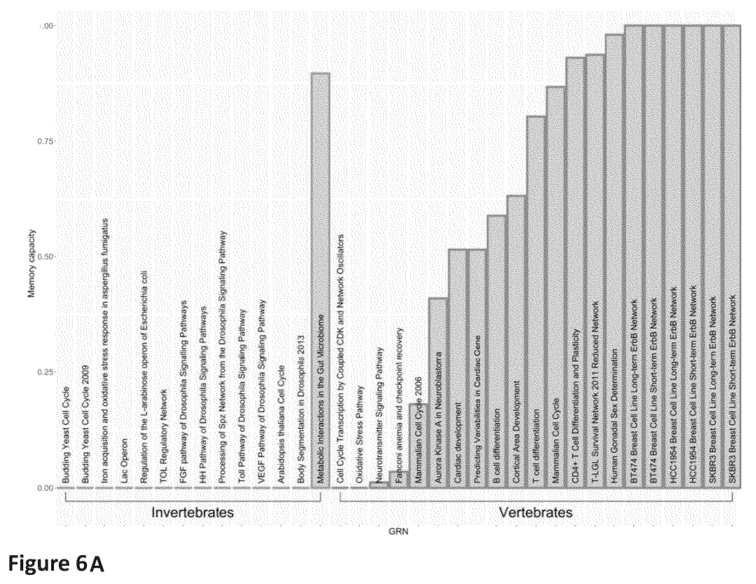

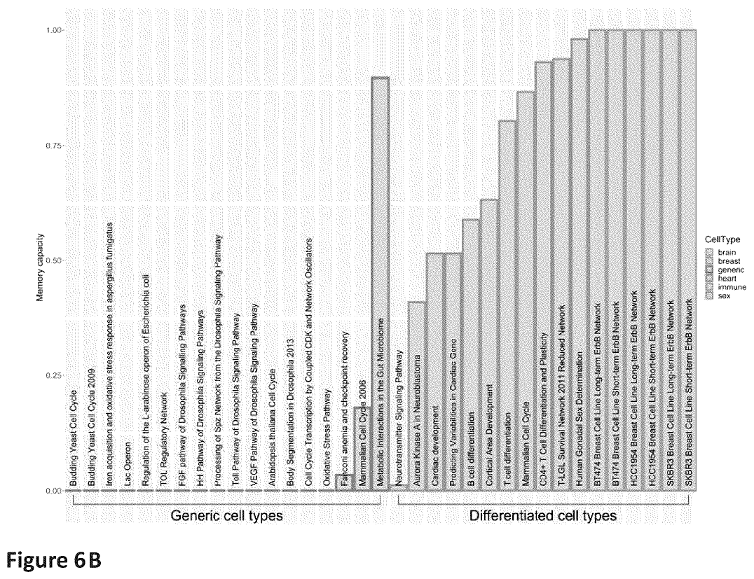

Is there any grouping of the different GRNs which reveals a pattern for when memory capacity is prevalent or not? We considered two simple categorizations of GRNs, one based on whether they belong to vertebrates or invertebrates, and the other based on whether they are associated with generic or metazoan differentiated cell types. We found that both the vertebrate/invertebrate distinction and the cell type features are excellent predictors of the existence of memory, as evidenced by their performance as classifiers (FIG. 6). Thus, we conclude that a diverse set of biological GRN structures exhibit various types of memory, which are especially highly represented among differentiated cells of vertebrate organisms.

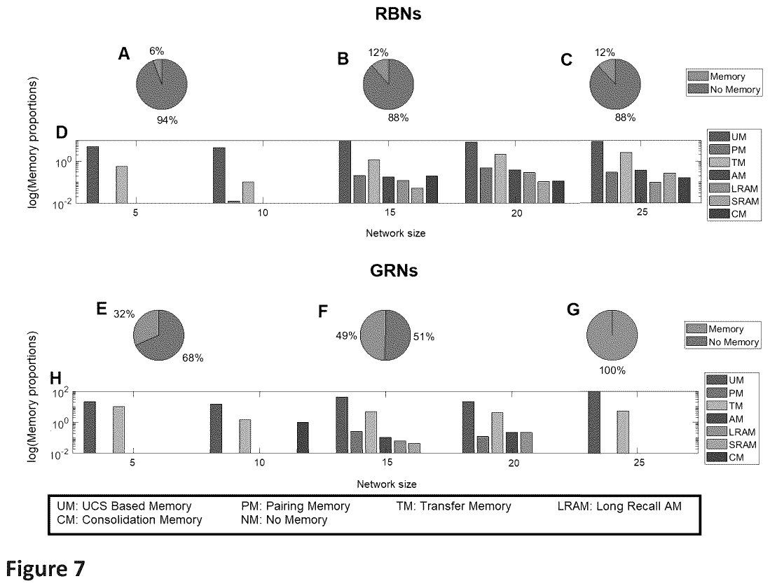

Memory type and frequency in the space of possible GRNs. Do larger networks in general have more memory capacity than smaller ones? In order to better understand the properties that underly memory in networks, we generated Random Boolean Networks (RBNs) to test different aspects of network structure. To determine how memory in RBNs changes with increasing network size, we created RBNs ranging in size from 5 to 25 nodes, with 100 RBNs generated for each size. We evaluated the pool of RBNs of each size separately to observe the change of average memory distribution with the increase in size. We found that that memory is less common in smaller RBNs (under 15 nodes in size, FIG. 7A, 7D) and restricted to UM and TM-type learning. The different types of memory start appearing in 15 node and larger RBNs. While UM dominates, all memory types were observed (FIG. 7B-D), with increasing amounts of the non-UM memory types at network sizes of 20 and 25 (FIG. 7C,D). Interestingly, in 15 and 20 node networks, LRAM is more common than SRAM, but in 25 node RBNs, SRAM dominates (FIG. 7B-D).

We then asked whether the same relationship between network size and likelihood of memory holds in biological networks. We grouped the 35 biological GRNs into 5 categories with network size 5-9 (2 GRNs), 10-14 (6 GRNs), 15-19 (14 GRNs), 20-24 (10 GRNs) and 25-25 (3 GRNs). We evaluated memory and present average memory distribution in the same manner as RBNs. We observed that GRNs have large amount of memories across networks, but, like RBNs, the percentage of networks with memory increases with network size. Availability and proportion of different types of memories in GRNs (FIG. 7E-H) are not entirely size dependent, although this relationship will become better quantified for biological networks when larger numbers of GRNs become available at different size ranges.

The memory profile of biological GRNs is unique. Do real biological networks' topologies offer more opportunities for memory dynamics than would be expected by chance in arbitrary networks of similar size and type? We generated 3500 “configuration models”-100 randomized versions for each biological GRN—and analyzed them for the presence and prevalence of each memory type. We then used statistical tests to compare these aggregate statistics to the memory profiles of the 35 actual biological networks, to determine whether GRNs of biological origin are in any way special with respect to memory capacity over what is provided by the generic properties of Boolean networks.

Given a certain type of memory in a GRN, we checked to see how the value fits into the probability distribution of the corresponding values of its ensemble. We calculated p-values (Supplement 1, Table 3) and conducted an outlier test (Supplement 1, Table 4). In each type of analysis, we obtained a matrix (35 GRNs each having 8 types of memory, including no memory). In the first case, each matrix element is a p-value [0,1]. We considered significance when p<0.05. In the second case, the value is binary (1: if the value is an outlier in its random ensemble; 0: otherwise). In either test, the percentage of success is relatively high for UM and TM compared to others.

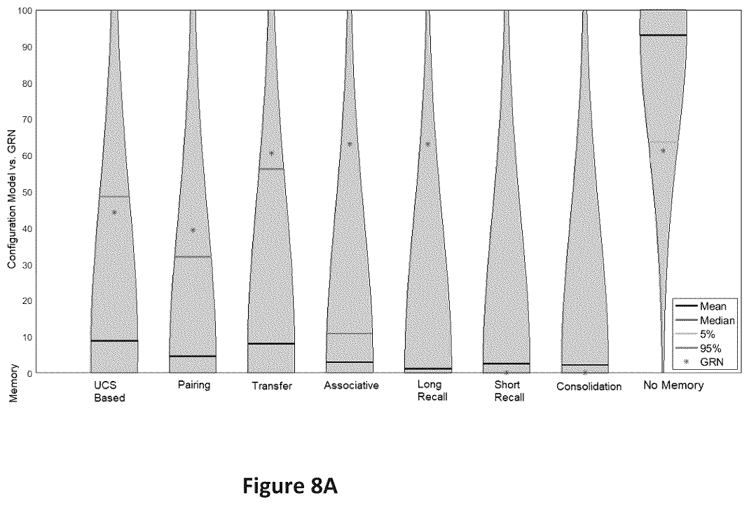

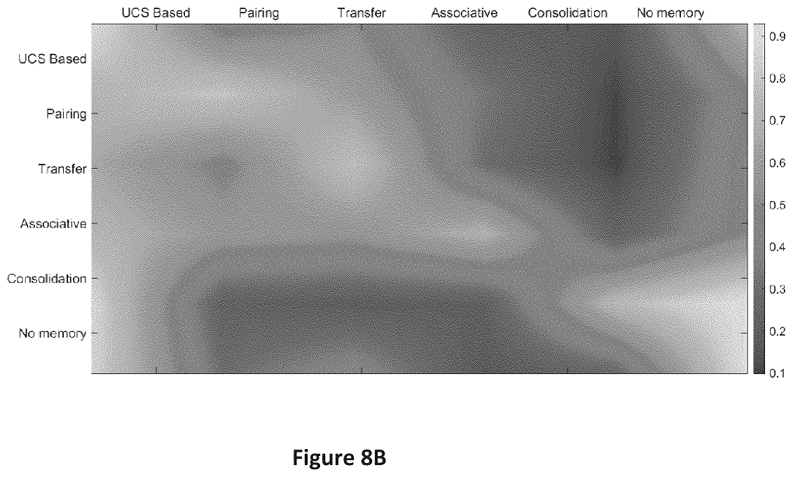

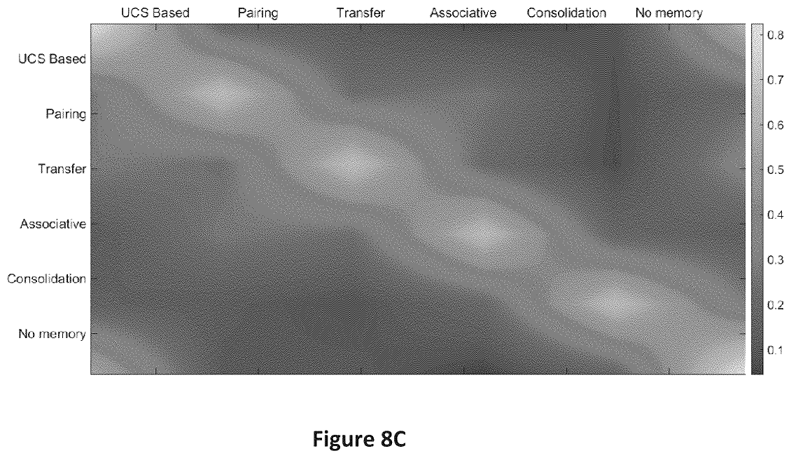

Further, we examined how each type of memories in a GRN fits into its random ensemble, visualizing the distribution of memories via violin plots (45). We found (FIG. 8A) that the incidence of memory-containing biological GRNs is generally unique with respect to possible GRNs, as it is outside the [5 95] percentile bars. Thus, we found that the data are not compatible with memory profiles in biological networks occurring solely as a consequence of the mathematical dynamics of Boolean networks (46). The fact that distribution of memories across real biological networks differs from that of randomized networks suggests that evolution has favored GRNs with specific memory properties. Our data do not distinguish between direct selection for memory in GRNs, or indirect selection in which memory is favored because it enables some other feature with selective advantage (e.g., plasticity of physiological response).

Since different kinds of memories have not before been rigorously defined for GRNs, or examined across the broad range of possible networks, it was not known whether memories tend occur in the space of GRN topologies independently, or whether certain GRN structures simultaneously predispose the network to multiple types of memories (perhaps distributed across different sets of CS/UCS nodes. Thus, we next sought to characterize the relationship between memories in a wide range of possible networks. Having generated a large number of configuration models, we asked whether the presence of one type of memory is statistically related to the likelihood of finding any other memories. We found that conditional entropy (specifying ordered correlation) between two types of memories in biological GRNs (FIG. 8B) is much higher than that of their randomized configuration models (FIG. 8C). Correlation between AM (especially LRAM), and any other memory type (leaving SRAM and CM) is especially significant. Biological GRNs show tight correlations between UM and TM. Moreover, in biological GRNs, PM is correlated both to UM and TM, but the correlation does not hold for the reverse direction, while CM has unidirectional correlation with UM. In the case of configuration models, the sub-categories of AM, named LRAM and SRAM showed correlation to AM. We conclude that the potential for forming different kinds of memories are not independent (that specific GRN architectures tend to support more than one kind of memory), and that the existence of some types of memory can be predicted solely based on the finding of other types of memory.

### Discussion

Numerous problems in biomedicine and fundamental life sciences face the inverse problem that affects all complex emergent systems: how do we control system-level behaviors by manipulating individual components? This problem is as salient for bioengineers and clinicians seeking to regulate gene expression cascades as for evolutionary developmental biologists seeking to understand how living systems efficiently regulate themselves (47, 48). An important direction in this field is the discovery of policies that use patterns of input (experiences) rather than hardware rewiring to achieve desired changes in network behavior. Is it possible to train gene regulatory networks, providing targeted patterns of stimuli to stably change their behavior at the dynamical system level, rather than rewiring network topology at the genetic or chromatin epigenetic levels? If so, this would take advantage of existing computational capabilities of the system and effectively offload much of the computational complexity inherent in trying to manage GRN function from the bottom up. Such approaches (49), if the GRN structures were amenable to them, would enable the experimenter, clinician, and indeed the system itself (in an evolutionary sense) to reap the same benefits as learning and training provide for neural systems. Thus, here we performed a systematic and rigorous analysis of memory in Boolean GRNs, an important model of gene regulation (50-52).

We first established a formalization of memory types for GRNs and implemented a suite of computational tests that reveal trainability in a given GRN. We next created and tested thousands of 2-node and 3-node networks to obtain the minimal networks exhibiting each type of memory. Then we tested different type of larger BNs from different sources. Our toolkit takes each network as input, generates the feasible UCS-R-NS combinations, evaluates the type of memory(s) in the current combination, counts the number of combinations for each type of memory (this counts combinations where no memory appeared) and returns these numbers to represent the memory landscape of the network. Overall, we tested 35 GRNs, 3500 configuration models (100 randomized models for each GRN) and 500 RBNs (100 each for networks of size 5, 10, 15, 20 and 25 nodes). We found a non-linear relationship of memory types with network size.

Different types of memory begin to appear in RBNs when networks reach 15 nodes in size. Larger networks of 25 nodes have stable quantities of memories and do not increase further. Thus, the structure of the GRN is more important that its mere size for implementing learning.

Prior work (11, 25) revealed associative memory and/or learning capabilities in different GRNs. We found the possibility of other types of memory beyond associative memory, and examined these dynamics broadly across a diverse set of GRNs. Using the data in Cell Collective, we tested 35 GRNs, 100 randomized models of each GRN (3500 in total) and 500 RBNs (100 of each size 5, 10, 15, 20 and 25). We observed that vertebrate GRNs have a much larger amount of memory than invertebrates. This is an important finding and may indicate that more complex developmental processes were evolutionarily favored with GRN architectures that exhibit more memory. Future work will examine additional GRNs as they become discovered within diverse taxa, to more fully appreciate the types of memory that exist across the tree of Life and the evolutionary significance of their distribution.

We further categorized the vertebrate GRN class into Cancer, Adult and Embryonic. We found a significant evidence of memories in Cancer and embryonic GRNs. The memory traces as the history of pathological and developmental states in cancer and embryos respectively may be stored as transcriptional regulations in GRN. AM has been identified in 5 GRNs out of 35 GRNs we tested. Among these GRNs, Aurora Kinase A in Neuroblastoma (vertebrate, cancer category) (53, 54), is the highest. Here, TPX2 (55) has appeared as a CS with a variety of genes or processes as UCS and R. CD4+ T Cell Differentiation and Plasticity (56), B cell differentiation (57) and Fanconi anemia and checkpoint recovery (58) (vertebrate, adult category) have AM. Human gonadal sex determination GRN of vertebrate-embryonic category also contains AM. Thus, AM represents 15% of our GRNs but is available in complex physiological, pathological and developmental regulatory processes.

Memories are more common in biological GRNs than in random networks. For instance, UM and TM are common in small GRNs and most GRNs contain these types of memory. Our results suggest that memory in a GRN strongly depends on the category of the GRN and the pathological and/or developmental processes in which they are involved, although many more GRNs filling out the space of processes will be useful in order to have a fuller picture of this relationship. Comparison of each GRN with its randomized configuration models indicated that GRN memory was an outlier compared in its randomized equivalent. Moreover, we found that only in real biological GRNs do different types of memory have distinct correlations between each other. AM, in is often highly correlated with UM and TM but not vice versa. Taken together, these analyses reveal several different ways in which biological networks are unique (and reflect richer properties than present simply by virtue of network dynamics in general (46, 59). Moreover, the specific associations between diverse memory types in biological GRNs form a complex and non-obvious relationship. These findings suggest the possibility that the evolutionary history of real biological organisms contained pressures (direct or indirect) favoring the existence of memory. Thus, an important area for future work is to identify GRN memory phenomena in vivo and ascertain their effects on selective advantage in terms of robustness, plasticity, and evolvability.

Numerous opportunities for subsequent work and for the interpretation of puzzling phenomena in biomedicine are suggested by these results. On the computational side, these analyses will next be extended to help understand the historicity of a wide variety of networks-continuous biological models (especially as well-parameterized biological ODE-type GRNs become discovered), protein pathways, and metabolic networks, as well as networks guiding the behavior of designed agents such as soft-body robots (60, 61). The existence of several different memory types could explain phenomena where combinations of drugs produce outcomes that are not predicted by chemical biology, treatments cease working, or well-tolerated compounds begin to have a different effect with time. Especially in the cancer and microbiome fields, these outcomes are typically thought to be due to population-level selection but could actually result from cellular or tissue-level learning within individual agents. GRN learning may also underlie some of the remarkable variability in drug efficacy and adverse effects that is observed across the population. An individual's response may be partially due to the GRN memories established over a lifetime of unique physiological experiences.

Immediate applications of our approach may include the use of associative memory to train tissues to respond to a neutral stimulus to mimic the effects of a potent drug that has too many side effects to use continuously. We will be testing this strategy in vitro and in vivo at the bench, targeting neuroblastoma and immune cell activation (FIG. 5). However, it is important to note that our methods are fully general and could be applied to identify learning in other types of important networks, from contact networks in epidemiology (62) to brain networks (63) to drug interaction networks (64). Thus, it is likely that the significance of finding trainability in network structures will extend well beyond biology. Overall, the discovery of memories in GRNs is a first step towards merging the approaches of network sciences with a cognitive science-based approach to regulation of complex systems (33, 65). It is likely that the discovery of memory, and perhaps future findings of other aspects of basal cognition in ubiquitous regulatory mechanisms, will provide important insight into the origin, self-regulation, and external control strategies over a broad class of dynamic systems in health sciences and technology.

### Materials and Methods

Models. Each GRN is represented as a standard Boolean Network (BN) model (66): a discrete dynamical system whose nodes represent the components of the system (e.g., genes or proteins) that can be in one of two states, namely 1 (ON) or 0 (OFF), and whose edges represent the regulatory interactions (activation/repression) among the nodes, dictating their states (67). The state of a BN is represented as a vector of the individual gene states, updated synchronously in discrete time-steps: the state of each gene at time t+1 is determined by a Boolean function of the states of its input genes at time t (68). A BN is simulated by initializing it with some state, then updating it to obtain the next state, and so on, for a specified number of time-steps. When a BN is simulated for a long enough time, it reaches an attractor state. An attractor may consist of a single BN state, known as a “point attractor”, or may consist of a set of states that the network cycles through, known as a “cyclic attractor.” A BN can have multiple attractors, and different inputs may lead to different attractors (68-71). In this work, we compute the memory profile of BNs in a manner that pays attention to its attractor states in order to avoid the effects of the transient dynamics on the analyses. This imposes a limitation on the size of networks considered because the larger the network, the longer it takes to reach an attractor. This transient length to reach an attractor depends on the Network Size (the number of nodes in the network) and the Edge Density defined as (Number of edges/Total number of possible edges). We found that the transient length (Supplement 2, Table 1) rises exponentially above 500 time-steps (a practical limit that we chose for this work) for networks of size larger than 25 with a biologically realistic edge density of 10% (Supplement 2, Table 2). As a result, we restricted ourselves to analysis of BNs of size<=25 to be able to exhaustively analyze all our networks.

Data: Biological and Synthetic Networks for Analysis. Our dataset consists of three kinds of BNs: 1) a set of 35 BN models of GRNs downloaded from an online model repository called Cell Collective (44), consisting of a maximum of 25 nodes each; 2) a set of 3500 BNs obtained by randomizing each GRN 100 times, known as “configuration models”; and 3) a set of 500 random Boolean networks (RBN). We generated a set of 100 configuration models for each biological GRN. For each configuration model we kept the number of nodes and the indegree distribution the same as the original GRN and randomized just the inputs to each node and the Boolean functions of each node. That is, each node in the configuration model has the same number of inputs, but the actual input nodes will be different compared to the original model. Similarly, each Boolean function in the configuration model has the same number of variables as the original but the Boolean operators are random, chosen from the set of elementary operators (AND, OR, NOT, etc).

To determine how the memory properties of networks vary with network size in general, we generated five sets of 100 RBNs each, of size 5, 10, 15, 20 and 25 nodes respectively. The edge density was set to max (10% of N², N−1), as the average edge density of the biological GRNs was found to be ˜10%. Unlike the configuration models, we generated an RBN by first randomly choosing unique source-target node pairs and assigning a directed edge between them such that the total number of edges satisfied the specified edge density, and then assigning random Boolean functions to each node. We generated a random Boolean function for a given node as follows. First, we considered the inputs of the node X that may consist of just one input (X itself or some other node,) or more than one input. In the case of the former, the Boolean function may take one of the following forms: ‘X=X’, ‘X=Y’ or ‘X=˜ Y’, where ‘˜’ represents logical NOT (invert) operation. If there are two or more inputs, such as (Y, Z), the Boolean function may take one of the following forms: (Y⊗Z), (˜Y⊗Z), (Y⊗˜Z) or (˜Y⊗˜Z), where ⊗ represents a Boolean operator randomly chosen from the list of Boolean operators (AND, OR and XOR). For more than two inputs, the Boolean functions would simply be larger compositions of the above. We then randomly applied NOT operation in the final or intermediate stages of the equation so that 50% of the nodes were affected.

Memory detection. We define different types of memories, characterized by a specific number and timing of the stimuli, as described below. To fully characterize the memory profile of a given BN, we exhaustively consider all “feasible” stimulus-response sets and enumerate all the memory types that each set elicits. By feasible combinations we mean the combination where UCS triggers R and NS does not trigger R. Generally, we report the amount of no memories counting the number of feasible combinations where any type of memory is unavailable. However, in 3 cases, (Arabidopsis thaliana Cell Cycle, Iron acquisition and oxidative stress response in Aspergillus fumigatus and Budding Yeast Cell Cycle 2009), we could not obtain any feasible combinations and thus considered the amount of “no memory” to be 100%. The set of all feasible stimulus-response combinations is a subset of all possible combinations, the cardinality of which is given by

$$P ( N , 3 ) = \frac{N !}{( N - 3 ) !} .$$

We compute a memory profile for each feasible combination by passing it through a series of detection steps (FIG. 3). We first let the BN settle on an attractor by initiating it with a state consisting of all “off” and simulating it for 500 time-steps. Then, we evaluate the memory of each combination via a sequence of steps picked from the following general recipe (the specific steps followed depends on the type of the memory being evaluated): 1) choose a stimulus set; 2) flip the state of the stimuli and fix them in that state, referred to as clamping; 3) simulate the BN for M time-steps; 4) record the state of R compared to its state prior to the clamping step; 5) unclamp the stimuli (allow them to update states), referred to as relaxation; 6) simulate the BN for M time-steps; 7) record the state of R compared to its state prior to relaxation; 8) choose a different stimulus set; 9) flip and clamp the stimuli; 10) simulate the BN for M time-steps; 11) record the state of R compared to its state prior to the clamping step 9; 12) relax the network; and 13) record the state of R. We deem a given stimulus-response combination as having elicited a specific type of memory if it satisfies the associated set of conditions:

UCS Based Memory (72): choose the stimulus set consisting of {UCS} in step 1, verify that R has flipped in step 3, and finally verify that R has not flipped in step 7. UM captures the idea that R may permanently remember changes in the activity of UCS.

Pairing Memory (PM): choose the stimulus set consisting of {UCS, NS} in step 1, verify that R has flipped in step 3, and finally verify that R has not flipped in step 7. PM captures the idea that R may permanently remember changes in the joint activities of UCS and NS. Even though the detection of PM is like AM, there are crucial differences (see AM definition below).

Transfer Memory (TM): choose the stimulus set consisting of {UCS} in step 1, verify that R has flipped in step 3, choose the stimulus set consisting of {NS} in step 8, and finally verify that R has flipped in step 11. TM captures the possibility that even though NS could not flip R initially, it may be able to do so after activating UCS, effectively transforming NS into CS.

Associative Memory (AM): choose the stimulus set consisting of {UCS, NS} in step 1, verify that R has flipped in step 3, choose the stimulus set consisting of {NS} in step 8, and finally verify that R has flipped in step 11. AM describes classical conditioning: after successful pairing of UCS and current NS, the NS is conditioned to become CS. This causes the NS to become CS and can be able to trigger R. In other words, we call it an AM if after successful pairing, NS can flip R.
a) Long Recall Associative Memory (LRAM): Following the AM steps, verify that R has not flipped in step 13 compared to its state prior to the relaxation step 12. LRAM captures the idea that R may permanently remember changes to the activity of CS.
b) Short Recall Associative Memory (SRAM): Following the AM steps, verify that R has flipped in step 13 compared to its state prior to the relaxation step 12. SRAM captures the idea that R may only transiently remember changes to the activity of CS.

Consolidation Memory (CM): choose the stimulus set consisting of {UCS, NS} in step 1, verify that R has flipped in step 3, choose the stimulus set consisting of {NS} in step 8, verify that R has not flipped in step 11, and finally verify that R has flipped compared to its state prior to the clamping step 9. CM captures the idea that even though associative conditioning may not immediately turn NS into CS, it may do so after relaxing the BN. Note that UM and PM are mutually exclusive, as are TM and {AM, CM} (see FIGS. 2,3 for details).

Mathematically, in an N node GRN, there may be

$${P}_{N}^{3}$$

such combinations. Here, we consider the current node as R if the R is stable over a certain period called Constancy Length during the relaxation phase of the network (see Supplement 2, Table 3). We coded the methodology in MATLAB 2019a.

### References for Example 1

1. Alvarez-Buylla E R, Balleza E, Benitez M, Espinosa-Soto C, & Padilla-Longoria P (2008) Gene regulatory network models: a dynamic and integrative approach to development. SEB Exp Biol Ser 61:113-139.
2. Huang S, Eichler G, Bar-Yam Y, & Ingber D E (2005) Cell fates as high-dimensional attractor states of a complex gene regulatory network. Phys Rev Lett 94 (12): 128701.
3. Peter I S & Davidson E H (2011) Evolution of gene regulatory networks controlling body plan development. Cell 144 (6): 970-985.
4. Davidson E H (2010) Emerging properties of animal gene regulatory networks. Nature 468 (7326): 911-920.
5. Singh A J, Ramsey S A, Filtz T M, & Kioussi C (2018) Differential gene regulatory networks in development and disease. Cell Mol Life Sci 75 (6): 1013-1025.
6. Qin G, Yang L, Ma Y, Liu J, & Huo Q (2019) The exploration of disease-specific gene regulatory networks in esophageal carcinoma and stomach adenocarcinoma. BMC Bioinformatics 20 (Suppl 22): 717.
7. Fazilaty H, et al. (2019) A gene regulatory network to control EMT programs in development and disease. Nat Commun 10 (1): 5115.
8. De Jong H (2002) Modeling and simulation of genetic regulatory systems: a literature review. Journal of computational biology 9 (1): 67-103.
9. Delgado F M & Gómez-Vela F (2019) Computational methods for Gene Regulatory Networks reconstruction and analysis: A review. Artificial intelligence in medicine 95:133-145.
10. Schlitt T & Brazma A (2007) Current approaches to gene regulatory network modelling. BMC bioinformatics 8 (S6): S9.
11. Herrera-Delgado E, Perez-Carrasco R, Briscoe J, & Sollich P (2018) Memory functions reveal structural properties of gene regulatory networks. PLOS computational biology 14 (2): e1006003.
12. Zagorski M, et al. (2017) Decoding of position in the developing neural tube from antiparallel morphogen gradients. Science 356 (6345): 1379-1383.
13. Lobo D, Solano M, Bubenik G A, & Levin M (2014) A linear-encoding model explains the variability of the target morphology in regeneration. Journal of the Royal Society, Interface/the Royal Society 11 (92): 20130918.
14. Baluška F & Levin M (2016) On Having No Head: Cognition throughout Biological Systems. Front Psychol 7:902.
15. Szabó Á, Vattay G, & Kondor D (2012) A cell signaling model as a trainable neural nanonetwork. Nano Communication Networks 3 (1): 57-64.
16. Turner C H, Robling A G, Duncan R L, & Burr D B (2002) Do bone cells behave like a neuronal network? Calcif. Tissue Int. 70 (6): 435-442.
17. Goel P & Mehta A (2013) Learning theories reveal loss of pancreatic electrical connectivity in diabetes as an adaptive response. PLOS One 8 (8): e70366.
18. Blackiston D J & Levin M (2013) Ectopic eyes outside the head in Xenopus tadpoles provide sensory data for light-mediated learning. The Journal of experimental biology 216 (Pt 6): 1031-1040.
19. Levin M (2014) Endogenous bioelectrical networks store non-genetic patterning information during development and regeneration. The Journal of Physiology 592 (11): 2295-2305.
20. Emmons-Bell M, et al. (2019) Regenerative Adaptation To Electrochemical Perturbation In Planaria: A Molecular Analysis Of Physiological Plasticity. iScience in press.
21. Sullivan K G, Emmons-Bell M, & Levin M (2016) Physiological inputs regulate species-specific anatomy during embryogenesis and regeneration. Commun Integr Biol 9 (4): e1192733.
22 Schreier H I, Soen Y, & Brenner N (2017) Exploratory adaptation in large random networks. Nat Commun 8:14826.
23. Soen Y, Knafo M, & Elgart M (2015) A principle of organization which facilitates broad Lamarckian-like adaptations by improvisation. Biol Direct 10:68.
24 Watson R A, Wagner G P, Pavlicev M, Weinreich D M, & Mills R (2014) The evolution of phenotypic correlations and “developmental memory”. Evolution 68 (4): 1124-1138.
25. Sorek M, Balaban N Q, & Loewenstein Y (2013) Stochasticity, bistability and the wisdom of crowds: a model for associative learning in genetic regulatory networks. PLOS computational biology 9 (8): e1003179.
26. Watson R A, Buckley C L, Mills R, & Davies A (2010) Associative memory in gene regulation networks. Artificial Life Conference XII, pp 194-201.
27. Durant F, et al. (2017) Long-Term, Stochastic Editing of Regenerative Anatomy via Targeting Endogenous Bioelectric Gradients. Biophys J 112 (10): 2231-2243.
28 Palm G (1980) On associative memory. Biological cybernetics 36 (1): 19-31.
29 Kohonen T (2012) Self-organization and associative memory (Springer Science & Business Media).
30. Rescorla R A (1967) Pavlovian conditioning and its proper control procedures. Psychological review 74 (1): 71.
31. Lee T I & Young R A (2013) Transcriptional regulation and its misregulation in disease. Cell 152 (6): 1237-1251.
32. Manicka S & Levin M (2019) Modeling somatic computation with non-neural bioelectric networks. Sci Rep 9 (1): 18612.
33. Manicka S & Levin M (2019) The Cognitive Lens: a primer on conceptual tools for analysing information processing in developmental and regenerative morphogenesis. Philos Trans R Soc Lond B Biol Sci 374 (1774): 20180369.
34. Fernando C T, et al. (2009) Molecular circuits for associative learning in single-celled organisms. Journal of the Royal Society Interface 6 (34): 463-469.
35. McGregor S, Vasas V, Husbands P, & Fernando C (2012) Evolution of associative learning in chemical networks. PLOS computational biology 8 (11): e1002739.
36. Gantt W H (1981) Organ-system responsibility, schizokinesis, and autokinesis in behavior. Pavlov J Biol Sci 16 (2): 64-66.
37. Gantt W H (1974) Autokinesis, schizokinesis, centrokinesis and organ-system responsibility: concepts and definition. Pavlov J Biol Sci 9 (4): 187-191.
38. Frey N, et al. (2019) Stevens-Johnson Syndrome and Toxic Epidermal Necrolysis in Association with Commonly Prescribed Drugs in Outpatient Care Other than Anti-Epileptic Drugs and Antibiotics: A Population-Based Case-Control Study. Drug Saf 42 (1): 55-66.
39. Tagkopoulos I, Liu Y C, & Tavazoie S (2008) Predictive behavior within microbial genetic networks. Science 320 (5881): 1313-1317.
40. Fernando C T, et al. (2009) Molecular circuits for associative learning in single-celled organisms. J R Soc Interface 6 (34): 463-469.
41. Blais A & Dynlacht B D (2005) Constructing transcriptional regulatory networks. Genes Dev 19 (13): 1499-1511.
42. Samal A & Jain S (2008) The regulatory network of E. coli metabolism as a Boolean dynamical system exhibits both homeostasis and flexibility of response. BMC Syst Biol 2:21.
43. Macneil L T & Walhout A J (2011) Gene regulatory networks and the role of robustness and stochasticity in the control of gene expression. Genome Res 21 (5): 645-657.
44. Helikar T, et al. (2012) The cell collective: toward an open and collaborative approach to systems biology. BMC systems biology 6 (1): 96.
45. Hoffmann H (2015) violin.m—Simple violin plot using matlab default kernel density estimation. INRES (University of Bonn), Katzenburgweg 5, 53115 Germany, hhoffmann@uni-bonn.de.).
46. Kauffman S A (1993) The origins of order: self organization and selection in evolution (Oxford University Press, New York) pp xviii, 709.
47. Crommelinck M, Feltz B, & Goujon P (2006) Self-organization and emergence in life sciences (Springer).
48. Karsenti E (2008) Self-organization in cell biology: a brief history. Nature reviews Molecular cell biology 9 (3): 255-262.
49. Pezzulo G & Levin M (2016) Top-down models in biology: explanation and control of complex living systems above the molecular level. J R Soc Interface 13 (124).
50. Lähdesmäki H, Shmulevich I, & Yli-Harja O (2003) On learning gene regulatory networks under the Boolean network model. Machine learning 52 (1-2): 147-167.
51. Martin S, Zhang Z, Martino A, & Faulon J-L (2007) Boolean dynamics of genetic regulatory networks inferred from microarray time series data. Bioinformatics 23 (7): 866-874.
52. Thomas P, Popović N, & Grima R (2014) Phenotypic switching in gene regulatory networks. Proceedings of the National Academy of Sciences 111 (19): 6994-6999.
53. Carmena M, Ruchaud S, & Earnshaw W C (2009) Making the Auroras glow: regulation of Aurora A and B kinase function by interacting proteins. Curr Opin Cell Biol 21 (6): 796-805.

54 Dahlhaus M, et al. (2016) Boolean modeling identifies Greatwall/MASTL as an important regulator in the AURKA network of neuroblastoma. Cancer Lett 371 (1): 79-89.
55. Kufer T A, et al. (2002) Human TPX2 is required for targeting Aurora-A kinase to the spindle. J Cell Biol 158 (4): 617-623.
56. Martinez-Sanchez M E, Mendoza L, Villarreal C, & Alvarez-Buylla E R (2015) A Minimal Regulatory Network of Extrinsic and Intrinsic Factors Recovers Observed Patterns of CD4+ T Cell Differentiation and Plasticity. PLOS Comput Biol 11 (6): e1004324.
57. Méndez A & Mendoza L (2016) A network model to describe the terminal differentiation of B cells. PLOS computational biology 12 (1): e1004696.
58. Rodríguez A, et al. (2015) Fanconi anemia cells with unrepaired DNA damage activate components of the checkpoint recovery process. Theoretical Biology and Medical Modelling 12 (1): 19.
59. Kauffman S A (1995) At home in the universe: the search for laws of self-organization and complexity (Oxford University Press, New York) pp viii, 321.
60. Auerbach J E & Bongard J C (2011) Evolving Complete Robots with CPPN-NEAT: The Utility of Recurrent Connections. Gecco-2011: Proceedings of the 13th Annual Genetic and Evolutionary Computation Conference: 1475-1482.
61. Bongard J & Lipson H (2007) Automated reverse engineering of nonlinear dynamical systems. Proc Natl Acad Sci USA 104 (24): 9943-9948.
62. Perra N, Gonçalves B, Pastor-Satorras R, & Vespignani A (2012) Activity driven modeling of time varying networks. Scientific reports 2:469.
63. Bassett D S & Sporns O (2017) Network neuroscience. Nature neuroscience 20 (3): 353.
64. Barabási A-L, Gulbahce N, & Loscalzo J (2011) Network medicine: a network-based approach to human disease. Nature reviews genetics 12 (1): 56-68.
65. Pezzulo G & Levin M (2015) Re-membering the body: applications of computational neuroscience to the top-down control of regeneration of limbs and other complex organs. Integr Biol (Camb) 7 (12): 1487-1517.
66. Herrmann F, Gross A, Zhou D, Kestler H A, & Kuhl M (2012) A boolean model of the cardiac gene regulatory network determining first and second heart field identity. PLOS One 7 (10): e46798.
67. Kauffman S, Peterson C, Samuelsson Br, & Troein C (2003) Random Boolean network models and the yeast transcriptional network. PNAS 100 (25).
68. Shmulevich I & Kauffman S A (2004) Activities and sensitivities in boolean network models. Phys Rev Lett 93 (4): 048701.
69. Serraa R, et al. (2007) Interacting Random Boolean Networks. Proceedings of ECCS07: European Conference on Complex Systems.
70. Xiao Y (2009) A Tutorial on Analysis and Simulation of Boolean Gene Regulatory Network Models. Current Genomics 10:511-525.
71. Veliz-Cuba A, Aguilar B, Hinkelmann F, & Laubenbacher R (2014) Steady state analysis of Boolean molecular network models via model reduction and computational algebra. BMC Bioinformatics.
72. Pietak A, Bischof J, LaPalme J, Morokuma J, & Levin M (2019) Neural control of body-plan axis in regenerating planaria. PLOS computational biology 15 (4): e1006904.

### Example 2

### Title: Re-Training Molecular Networks: a New Path Toward the Biomedicine of Cancer and Regeneration Revealed by a Basal Cognition Approach

### Executive Summary

We will develop theory and perform biological experiments to test the hypothesis that evolutionarily ancient (pre-neural) cellular mechanisms, such as molecular networks could exhibit learning (a basic aspect of primitive cognition). We will leverage the tools of behavior science, using experiences (specific temporal regimes of stimulation), to control outcomes in gene regulatory networks (GRNs), with major advantages over traditional molecular rewiring (purely mechanist) approaches. One example is associative learning, where an effective drug, too strong to be used in patients, is paired with a neutral one, forming a kind of Pavlovian conditioning in the molecular pathway that will enable the same desired response to be triggered by the neutral drug alone (enabling effective repurposing of numerous drugs throughout the pharmaceutical industry). Other applications include understanding pharmacoresistance as a kind of behavioral habituation, and developing methods for predicting what kinds of drug stimulation would reverse it. Our goal is to show how the use of a basal cognition framework in Genetics can lead to novel therapeutically-relevant interventions. We will perform a combination of computational modeling and experiments to show a unique proof-of-principle strategy based on a “mind everywhere” framework to control complex system-level outcomes of great significance to biomedicine. This will avail the field of the study of Intelligence with an exciting and tractable new model system for basal cognition (memory and learning in molecular networks), and will provide a link for that emerging field to impactful, practical outcomes. Our progress will engage the biomedical community's immense intellectual and funding resources in the question of unconventional substrates for intelligence, greatly enhancing the ability of our community to make progress. In order to push the risk-averse pharmaceutical industry to engage with the basal cognition community, proof-of-concept data must be obtained; this project is designed to break the catch-22 by seeding a critical set of results that will unlock partnerships with biotech.

Our specific aims are to produce a device+software that not only answer a specific biological question, but form a versatile platform for future advances by many other groups, enabling the discovery of training protocols for any type of cell, for other biomedical purposes. The big question we address is: how can the tools of computational behavior science be brought to bear on molecular networks to solve key open problems in physiology and medicine? We hypothesize that the basic paradigm of genetics must be augmented with the tools of behavioral science in order to make full use of the plasticity of the genomically-encoded hardware of cells. This is important for two reasons. First, it will provide a proof of concept of how to use basal cognition approaches for a practical purpose beyond basic science and philosophy of mind—a fascinating extension of cognitive concepts to genetics. Second, it will give rise to new biomedical strategies, alleviating human suffering and bringing the biotechnology industry in as stakeholders in the field of basal cognition.

Humanity has been training animals for millennia, without knowing anything about what is in their heads or how it works. This highly efficient approach works because we correctly identified animals as learning agents, which allows us to offload a lot of the computational complexity of any task onto the system itself, without micromanaging it mechanistically (bottom-up control). Molecular pathways have not heretofore been exploited as that kind of learning system, resulting in powerful limitations to modern medicine despite the deluge of molecular data being produced. We will train molecular networks instead of genetically rewiring them to: 1) advance our understanding of novel embodiments of (primitive) minds, and 2) show how this approach has important, practical benefits for biomedicine.

We will address key needs and knowledge gaps by 1) producing a new device and computer software that will 2) uncover effective mechanisms to address problems of drug toxicity, pharmacoresistance, sensitization, and unpredictability in cellular systems (with a focus on cancer physiology), thus 3) addressing a specific pressing biomedical need. Our project will specifically test the hypothesis that associative conditioning (and several other learning types) exist as a practically exploitable phenomenon in GRNs. We anticipate specific outputs of software, publications, presentations, and partnerships with the biotech industry that will have strong impact via outcomes that include: 1) a novel understanding of unconventional (non-neural) substrates of primitive mind, 2) better understanding of the relationship between dynamical systems and cognitive approaches (soft emergence), and 3) engagement of the massive resources of the pharmaceutical industry into the field of basal cognition via drug pathway training as a way of repurposing and improving the breadth of indications for old and new drugs. The implications for Genetics of memory studied in molecular networks range from fundamental understanding of evolvability and the relationship between genome and function, to applications in molecular medicine.

### Project Details

### Statement of Significance

This project is important because 1) it will deliver a practical, powerful new model system to advance the understanding of scaling of mind from humble molecular origins, and 2) it will establish a milestone in the understanding of basal cognition by fusing advances in this emerging field with progress in a pressing biomedical problem. The latter is an essential step because it would show practical utility for questions that previously have been largely marginalized by those who focus entirely on classical (advanced brain) neuroscience systems and workers in molecular biology who focus almost exclusively on reductive approaches. Success of this project would give rise to a powerful new synergy of two fields: molecular medicine and basal cognition, not only pushing questions of substrates of mind beyond philosophy and into tractable empirical work, but also providing the basal cognition community with important new allies. The biomedical and pharmaceutical industries have very deep pools of human talent and financial resources, which could be used to exponentially increase the impact of work in unconventional approaches to various types and scales of minds, if early philanthropic investment in the disclosed work de-risked the field by producing a critical set of proof-of-principle results and methods.

Our project has high relevance and significance to a number of constituents. Workers in pharmaceutical and biomedical fields (both commercial and academic) would be able to use this new approach to improve the utility of existing drugs and repurpose novel ones. They could readily augment their drug discovery and testing pipelines to go beyond highly limiting “pick the best dose and keep it constant” method, using our new method to create and identify much more powerful timed presentation strategies (importantly, we will provide not only a new conceptual strategy but actual software and a physical device to enable and facilitate adoption). Human patients would benefit from the ability to associate harmless drugs with powerful ones that have undesirable side-effects, and other improvements in healthcare that will result once we understand how to exploit the innate intelligence of the body's physiological control circuits. Basic scientists in molecular biology and genetics will be able to apply other tools from the behavioral and cognitive sciences to their work, going beyond limiting mechanistic tools to exploit the novel “software-like” properties of their favorite pathways and networks.

Our goal is to promote human flourishing by establishing a roadmap for a new kind of biomedical strategy that rests on recognizing and working with a new kind of intelligence—the primitive intelligence within cells. The project is a tool-building effort, with impact well beyond our specific applications; we want to add a cognitive dimension to the existing emphasis on mechanistic approaches to control of complex systems. It is designed to produce the enabling technology and proof-of-principle data which will make it possible for academic laboratories and industry (bio-pharma world-wide) to pursue a whole new way to intervene in disease. We seek to provide a new context—a new way of thinking about the problem of cell dysregulation, that will greatly promote innovation as others take up this approach and apply it to a huge diversity of processes and purposes: “biomedical interventions leveraging the collective intelligence of different levels of organization of living systems”. The first practical tools in this area will also have implications for bioengineering (creating useful synthetic living machines), machine learning (by showing a new kind of non-neuromorphic architecture which can be readily implemented in devices and improved), and other fields beyond biology in which all kinds of networks need to be controlled.

This project is fundamentally about identifying primitive cognitive capacities in areas of science normally thought to be paradigmatic cases of mechanism. Thus, it serves as a proof of principle for unifying two approaches that are often thought to be incompatible: is a given system a “mere mechanism” or does it have agency? Prior work in the philosophy of science and mind has claimed that these are compatible, but we will test a unique, practical example of how to identify and exploit a degree of cognition in a canonical mechanist framework. Our recent analyses suggest that pathway networks should be able to “learn” from experience, and can thus be trained in ways readily recognizable to workers in behaviorist or cognitive science. Our project will demonstrate how a mechanistic framework (the GRN formalism) can be integrated with the perspective of unconventional intelligences, in a way that exploits the advantageous qualities of each approach. It is an example of how to find primitive Minds in systems that were not yet known to be substrates for cognitive capacity such as learning.

Finally, the project is about validating these ideas empirically, in a way that demonstrates their utility in biomedicine. It would significantly revise thinking in the fields of genetics and molecular biology by showing them the possibility, and practical value, of adding a cognitive perspective to current mechanistic models. A new frontier exists at the interface of reductive and holistic approaches to networks. A synthesis has been sought for many decades by both philosophers and those who seek to predict and control complex systems. We will implement a novel research program to significantly advance this effort.

We should point out that it is not claimed here that this training paradigm is the only, or the ultimately best, way of approaching biomedical goals. We view it as a strong complement to today's strategies based on rewiring, with advantages over past views of the problem, but are completely open to the fact that future work might identify an even better way to address these complex systems' behaviors. In any case, our work will seek a unification, using soft emergence as a way to think about how dynamical systems models and memory models can both be true, complementary descriptions of a multi-scale problem of prediction and control. One specific hypothesis we will test is that the differences between these approaches can be visualized as learning being able to operate in a simpler, coarse-grained “reward space” that the more complex, high-dimensional space that strategies in dynamical systems micro-management need to traverse in order to be successful.

Upon completion, this project will result in new insights into the learning-like plasticity of molecular biology mechanisms within cells, and the availability of software, device design specs, and protocols that many members of several communities could use to embark on a wide range of new studies. This project will lead to broader impacts that strongly transform both the field of basal cognition (by providing a new community with many useful resources) and that of molecular and genetic medicine (by providing a new way to address disease states using already-available compounds). The work will potentiate the investments already made into drug discovery, genomics, and molecular biology, in addition to opening additional opportunities in applying cognitive approaches to many other contexts. The implications for Genetics, of memory studied in molecular networks, range from fundamental understanding of evolvability and the relationship between genome and function, to applications in molecular medicine.

Our computational, quantitative analysis predicts a wide range of clinically-important behaviors in existing network models, which can be exploited if we treat these models as a primitive cognitive agent (a simple biological system that can be trained with appropriate behavior shaping experiences, not only hardware rewiring). One of the most remarkable is associative learning (Pavlovian conditioning). Much as a dog may learn to salivate when hearing a bell (a previously neutral stimulus) if the bell and meat are presented together a few times, some gene regulatory networks should activate a desired response when a neutral node is triggered if that node had been previously triggered together with another node that is alone sufficient to cause the response. This is significant because it means, for example, that the many drugs that are effective but too strong to be used in human patients, could be repurposed if one could condition a neutral, harmless drug with a few presentations together, and subsequently (for some period of time) use the neutral stimulus alone to trigger the desired response without having to use the toxic agent (e.g., oxphos inhibitors, DNP, rapamycin, acetylcholinesterase inhibitors, systemic steroids, etc.). This could allow us to repurpose huge numbers of compounds which otherwise “failed” clinically due to toxicity (or due to pharmacoresistance, which our paradigm also shows how to overcome). Additional impacts of computationally understanding memory in pathways include the abrogation of pharmacoresistance over time, and personalization/prediction of drug efficacy and safety and prediction of failure in static dose regimes. However, current molecular medicine approaches are firmly entrenched in a view of pathways as mechanisms that must be controlled “bottom up” and a search for one constant “correct drug dose” for each patient. We will test at the bench several novel hypotheses and create a “discovery engine” platform that will enable the community to identify ways to train networks that are too complex to micromanage bottom-up for desired system-level behavior. This work will implement the essential de-risking and proof-of-concept discovery that is necessary before traditional funding sources (NIH, biopharma, etc.) move into this area.

The project is also about understanding the “many to one” transition in cognitive science. All Selves are made of parts, starting with components specified by Genetics; how do individual parts work together to give rise to an emergent entity that has memories (and goals, preferences, etc.) that belongs to a higher-level agent and not to any of the parts alone? This has been studied by neuroscientists in the context of cells, and by those working on swarm intelligence in the context of animal or robotic collectives, but still remains a major knowledge gap. We show how a collection of molecules in a network can work together as a special kind of dynamical system which can process a history of experiences (inputs, stimuli) and gain an associative or other kind of memory that belongs to no individual molecule but to the system as a whole. Our work will produce and analyze an extremely tractable “minimal system” in which to begin to understand how larger kinds of Minds emerge from the activity of their components.

### General Introduction and Perspective

Humanity has been training animals to perform complex tasks for millennia, without knowing anything about what is in their heads or how it works. This highly efficient approach works because we correctly identified animals as learning agents, which allows us to offload a lot of the computational complexity of any task onto the system itself, without micromanaging it from the bottom up. What other systems might this powerful strategy apply to? Molecular pathways have not heretofore been exploited as that kind of system, resulting in powerful limitations to modern medicine despite the deluge of molecular data being produced.

We will train molecular networks instead of genetically rewiring them, and use this to: 1) advance our understanding of novel embodiments of (primitive) minds, and 2) show how this approach has important, practical benefits for biomedicine. Our preliminary computational results [1] suggest that molecular (gene regulatory, protein pathway, and metabolic) networks are not mechanisms with fixed behavior, but rather can learn from experience. That is, their future behavior is a function of what inputs they have experienced in the past, and molecular networks can be treated as if they were “neural networks”, with all of the powerful functional implications of such an isomorphism. This means that the advances of cognitive neuroscience and behavioral science could be brought to bear to identify specific regimes of stimulation of molecular pathways (via pulsed drug, light, fluid flow or other modalities) that result in desired behavior of the network without needing to change the cellular hardware via genomic editing or gene therapy. We will train molecular networks instead of rewiring them, and use this to: 1) advance our understanding of novel embodiments of (primitive) minds, and 2) show how this approach has important, practical benefits for biomedicine (for example, enabling the use of a myriad drugs that “failed” in simplistic assays with a constant, not time-dependent, exposure method).

In a preferred embodiment, nicotine is administered to a subject through a fluid flow device, wherein the nicotine triggers a response in the GRN and a corresponding therapeutic response in the subject when the nicotine is administered to the subject without fluid flow, and wherein the fluid flow does not trigger a response in the GRN and a corresponding therapeutic response in the subject when the fluid flow is administered to the subject without nicotine; and subsequently repeating the nicotine with fluid flow treatment one or more times until the fluid flow triggers a response in the GRN and a corresponding therapeutic response in the subject when the fluid flow is administered to the subject without the nicotine, thereby treating the disease or disorder characterized by the GRN in the subject by administering the fluid flow.

The current molecular medicine approach is highly mechanist; despite the understanding of the importance of complexity, emergence, noise, and environment, it is still the case that molecular pathways are largely investigated and controlled bottom-up, pursuing ever higher-resolution views of molecular function, hoping to identify specific changes that can be forced at the molecular level. This approach limits the impact of genetics and genome editing, because aside from single-gene diseases and a few other simpler cases, it is generally unclear what parts of the genome to edit (and how) to get complex, desirable outcomes at the system level. This task is akin to programming computers by physically rewiring them. In the 1940's and 1950's, this is how programming was done, but computer science quickly learned that we can make massive progress (giving us the information technology revolution) if we focus on reprogrammability of the hardware, not rewiring. We now control computer devices via experiences (stimuli, signals-produced by a keyboard or similar “sensory” input node in the circuit network), which is an incredibly powerful tool. Advances in our lab and others' suggest that biological tissues are also plastic and reprogrammable; treating them exclusively as “clockwork” mechanisms (when in reality they are cellular collectives with swarm intelligence that can be exploited) is limiting progress and can be complemented by a different approach to unlock important capabilities.

In order to really understand the behavior and capabilities of gene-regulatory and other networks, it is essential to determine their innate computational capabilities, and ascertain what type of intervention approach would give the most control for the least effort needed. It is commonly assumed that GRNs, a paradigmatic case of molecular mechanism, have to be managed bottom-up—as purely mechanical systems. Because the idea of GRNs being an agent capable of learning is novel and surprising in most communities, we first address a set of conceptual issues.

We will test whether the correct level of agency with which to treat this (or any other) system cannot be determined by armchair philosophy but must be established by experiments that reveal which kind of model and strategy provides the most efficient predictive and control capability over the system. In this view, agency is a continuum and the optimal position of a system on this spectrum is determined empirically. A standard methodology in science is to avoid attributing agency to a given system unless absolutely necessary. The mainstream view (e.g., Morgan's Canon) is that it's too easy to anthropomorphize systems with only apparent cognitive powers, in favor of models focused on mechanistic, lower levels of description that eschew any kind of teleology or mental capacity [2,3]. We seek to complement the rich history of philosophical debates on reductionism and mechanism with an empirical, engineering approach that identifies and exploits the primitive intelligence components of pathways like gene-regulatory networks (GRNs).

One of the key Big Questions spanning philosophy, the science of Intelligence, and engineering is that of agency: what kinds of material embodiments can (or must) be treated as Selves with preferences, memories, goals, etc.? This is important not only to foundational issues of philosophy of mind and ethics, but also to scientific efforts in artificial life, machine learning, exobiology, etc. The emerging field of “Basal Cognition” [4,5] expands traditional brain-focused approaches to encompass the phylogenetic origins of intelligence and its capacities. It seeks to develop tools for recognizing agency (and characterizing its capabilities) in novel forms, which may be very different in scale and kind from the familiar intelligences of higher animals. This framework holds that primitive cognitive functions (plasticity, learning, anticipation, etc.) can be embodied by many different kinds of processes, not only the familiar context of animal brains. What kinds of unconventional media support primitive forms of mind and how would we recognize them?

On this view, it is just as bad to under-estimate the level of mentality of a system as to over-estimate it. Specifically, under-estimating the capacity of a system for plasticity, learning, having preferences, representation, and intelligent problem-solving greatly reduces the toolkit of techniques we can use to understand and control its behavior. As a simple example, consider the task of getting a pigeon to correctly distinguish videos of dance vs. those of martial arts. If one approaches the system bottom-up, one has to implement ways to interface to individual neurons in the animal's brain to read the visual input, distinguish the videos correctly, and then control other neurons to force the behavior of walking up to a button and pressing it. This may someday be possible, but not in our lifetimes. In contrast, one can simply train the pigeon [6]. This highly efficient trick works because we understood something about the kinds of stimuli that can be used to leverage the animal's innate learning capacities. What other systems might this remarkably powerful strategy apply to?

A gradualist approach considers the many ancient contexts in which life had to perform problem-solving before advanced mammalian brains appeared. On this view, it is incorrect to look for a clear bright line that demarcates “true” cognition (such as that of humans, great apes, etc.) from metaphorical “as if cognition” (fictitiously applied to other life forms). Instead of a binary dichotomy, we envision a continuum of phylogenetic advancement in information-processing capacity which has phase transitions to new capabilities but is nevertheless a continuous process that is not devoid of proto-cognitive capacity before complex brains appear. This framework asks “how much” and “what kind of” cognition any given system might manifest if we understood how to exploit it. Our other work along these lines suggsts a multi-axis option space that enables direct comparison of the proto-agency of all sorts of systems of varied material implementations and origins [7, 8].

This has two advantages. First, it takes evolution seriously, including recent advances on the phylogenetic origins of cognitive capacities and the high conservation of both molecular mechanisms and algorithms between their humble somatic origins and advanced modern brains [4, 5, 9]. This allows us to deploy tools that have been successfully used in familiar animals to study plasticity and learning, in novel contexts such as control of molecular pathways. Second, it provides a clear path forward requiring empirical approaches to prediction and control, to specify the appropriate (not unique, but optimal) level of agency for any given system. This is akin to Dennett's “Intentional Stance” [10, 11], but with an emphasis on practical experimental approaches that have many applications to fields such as genetics, biomedicine, artificial intelligence. It is essential to develop these applications not only to gain basic insights, but to provide empirical evidence that basal cognition is an important field and thus attract talented workers from other areas into the search for an understanding of diverse “mind as it can be”.

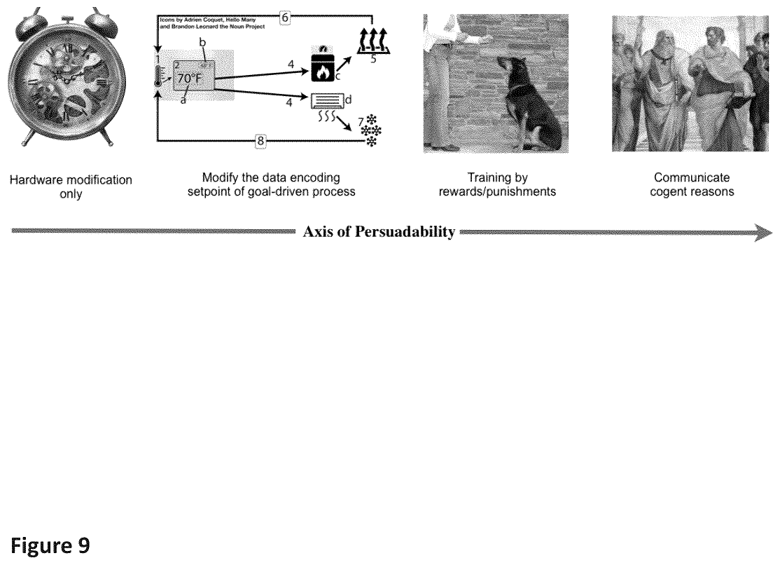

Our framework, for the surprising hypothesis that a genetic network can learn, is an “axis of persuadability”: a (multi-dimensional) continuum on which any system can be placed, with respect to what kind of strategy is optimal for prediction and control (FIG. 9). On the far left are the simplest physical systems, e.g. mechanical clocks. These cannot be persuaded, argued with, or even rewarded/punished-only physical hardware-level “rewiring” is possible if one wants to change their behavior. On the far right are human beings (and perhaps others to be discovered) whose behavior can be radically changed by a communication that encodes a rational argument that changes the motivation, planning, values, and commitment of the agent receiving this. Some of these systems are so complex that they can even fall prey to a “thought that breaks the thinker” (e.g., existential or skeptical arguments that can make one depressed or even suicidal, Gödel paradoxes, etc.)—massive changes can be made in those systems by a very low-energy signal because it is treated as information in the context of a complex host computational machinery. Between these extremes lies a rich panoply of intermediate agents, which can be controlled by signals, stimuli, training, etc. They can have some degree of plasticity, memory (change of future behavior caused by past events), various types of simple or complex learning, anticipation/prediction, etc. Some may have preferences, which avails the experimenter of the technique of rewards and punishments—a more sophisticated control method than rewiring, but not as sophisticated as persuasion (the latter requires the system to be a logical agent, able to comprehend and be moved by arguments, not merely triggered by signals).

This is not meant to be a scala naturae that aligns with any kind of “direction” of evolutionary progress-evolution is free to move in any direction in this option space of cognitive capacity; instead, this scheme provides a way to formalize (for a pragmatic, engineering approach) the major transitions in cognitive capacity that can be exploited for increased insight and control. The goal of the scientist is to find the optimal position for a given system. Too far to the right, and one ends up attributing hopes and dreams to thermostats or simple Als in a way that does not help with prediction and control. Too far to the left, and one loses the benefits of top-down control in favor of intractable micromanagement. Molecular control mechanisms such as GRNs have always been assumed to be the mechanistic, clockwork-like systems and treated as such (via micromanagement of network topology via genetic modification). Such a priori assumptions are unwarranted and entail a huge opportunity cost for the fundamental understanding of the phylogeny of intelligence and for biomedicine. They need to be tested empirically; our hypothesis is that regulatory pathways are in fact somewhere on the middle of the persuadability spectrum which, if true, opens the door to an entirely novel and powerful set of strategies for their manipulation (and for building novel proto-cognitive agents).

Our goal in this project is two-fold. First, we seek to establish a practical “killer application” (in computer parlance) that clearly demonstrates proof-of-principle of key ideas in the basal cognition field. We want to show how a non-teleophobic, non-binary approach to the search for mind in unconventional media leads to practical advances that quantifiably recommend this view over competing strategies (e.g., a default to mechanistic, reductionism and inappropriate deployment of Occam's razor). Second, we seek to solve a pressing problem in molecular medicine, which will complement mainstream work on genomic editing, and serve as an enabling technology for transformative advances in regenerative medicine.

### Specific Overview: Training Molecular Networks

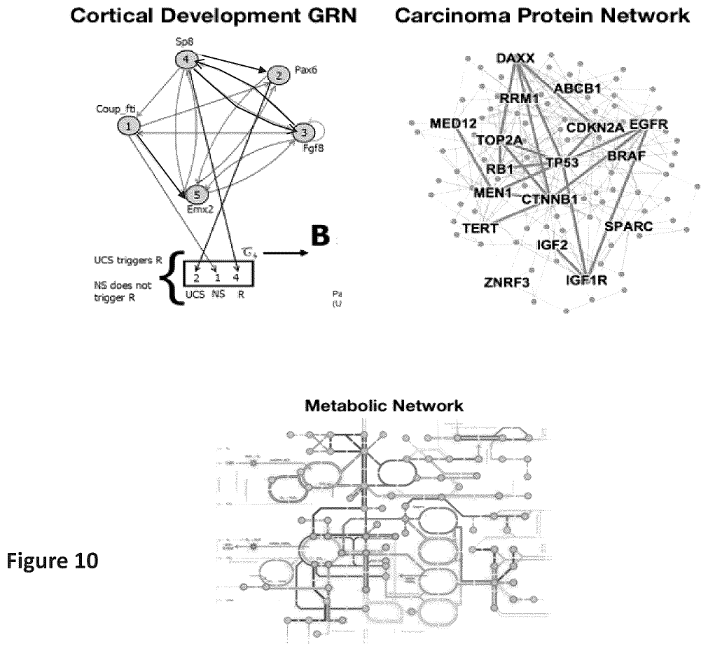

A key formalism in modern molecular biology and medicine is that of a network: gene-regulatory networks, protein networks, and metabolic networks consist of nodes connected by functional relationships (e.g. activation/repression) in some sort of topology (FIG. 10). For example, gene regulatory networks (GRNs) are key drivers of embryogenesis, and their importance for guiding cell behavior and physiology persists through all stages of life [12, 13]. Understanding the dynamics of GRNs is of high priority not only for the study of developmental biology [14, 15], but also for the prediction and management of numerous disease states [16-18].

The molecular network paradigm is an ideal example of the mechanist approach: it is hoped that by learning to manage the connections of molecular networks, all the apparent intelligence [19, 20] of morphogenetic homeostasis (regulative development, regeneration, etc.) will be explained by this very straightforward, deterministic type of system. Of course, sophisticated approaches also include stochastic components (noise), biomechanical forces, etc. but the basic assumption is that directly modifying the hardware—the network topology—is the path to system-level control over morphogenesis and disease.

While these paradigms are clearly useful, they are limited in scope because of the inverse problem: the difficulty of inferring what changes need to be made to the subunits, in order to drive desired changes in large-scale, system-level behavior [19, 21]. What changes must be made to the simple, local rules that individual termites follow, if one wanted them to make a nest with 2 chimneys instead of one? What changes must be made to the rules of the Game of Life cellular automaton if one wanted a glider that moved in a different path? What genes should be up- or down-regulated in a cell in order to create a hand or an eye with an appropriate anatomy? All of these are the same problem: the difficulty of inferring low-level changes that will drive desired large-scale, system-level states. Deterministic chaos and complexity theory have made it very clear why bottom-up control of even simple systems (e.g., 3-body problem) can be practically impossible. Evolution solved this problem by utilizing biological subsystems that do not have to be controlled by solving the inverse problem [21], but rather by training and experience [22].

In molecular medicine, this limitation may give rise to a “genomics winter” paralleling the AI winter, a period of stagnation after most of its tractable problems had been solved and new techniques were not yet in hand that lasted for more than two decades. [23]. In the next few years, the genetics community will solve the mechanics problems of genomic editing and gene therapy (being able to cleanly modify DNA in vivo) and stem cell biology (being able to produce any cell type from a parent stem cell). But then, beyond the low-hanging fruit of single gene and single cell diseases, how would we know which genes to edit to re-grow a limb or repair a craniofacial birth defect, or how to assemble individual stem cell progeny into a hand or a synthetic living machine with a desired structural and functional spec? Making good on the promises of regenerative medicine, and fully deploying the power of existing genetic and cell biology technology requires complementing the current mechanist paradigm with an information-focused approach that exploits collective intelligence of cellular and subcellular components to deploy techniques from the middle portion of the persuadability scale: training, reinforcement, and other approaches taken from behaviorist and cognitivist toolkits that were previously reserved for animals with brains.

Evolution discovered long ago that bottom-up mechanical control is insufficient for the kind of plasticity needed to effectively deal with a challenging world. It solves the inverse problem on the geological timescale via a massively-parallel genetic search algorithm, while solving it on the scale of an individual's lifetime via a combination of bottom-up emergence and top-down homeostatic plasticity. For example, tadpoles with eyes moved to their tails can see—not requiring generations of selection to adapt to this body configuration [24, 25]. Tadpoles with their faces artificially rearranged still make normal frog faces as each organ moves through un-natural paths to make the proper target morphology [26]. Thus, evolvability and highly adaptive anatomical remodeling work because cells and tissues exploit massive plasticity, as their activity is guided by both past experience and homeostatic setpoints [19, 27-29]. In the same way that a planarian body can be permanently shifted to a 2-headed regenerative form by transient external changes to its bioelectric pattern memory [30-32], a molecular network has the capacity to dynamically respond to environmental stimuli to stably change how it reacts in the future. We can harness that innate plasticity/adaptability, guiding a system to shift to desirable configurations by experiences, instead of trying to force it by bottom-up micromanagement.

### Preliminary Data: De-Risking a Focus on Cellular Plasticity

We have recently developed theory around the concept that flexible problem-solving in anatomical morphospace is an ancient evolutionary capacity that served as a precursor to brain-specific behavioral plasticity observed in more advanced life forms [9, 19, 20, 33-39]. This served as the background for functional approaches to identify these capacities (like training) in developmental mechanisms (like GRNs) and a focus on deriving novel predictions and capabilities from our models of physiological plasticity. The following demonstrate that we have a track record of success in components relevant to the experimental validation of these ideas:

1) We identified novel roles for neurotransmitters [40-44] and ion channels [45-51] as ancient, pre-neural machinery involved in cells making decisions during embryonic patterning. 2) We have shown non-neural bioelectricity as a molecular mechanism that underlies the ability of all cells, neurons and others, to form collectives that process information that scales to the goals of multicellular entities—a kind of collective intelligence [52-55]. 3) We have demonstrated the ability to computationally model the plasticity of cells in a way that directly informs the design of molecular/biophysical interventions that over-ride genomic defaults such as mutations in the Notch gene [56] or in KRAS oncoproteins by providing cells in vivo with transient physiological experiences [57-60]. This work shows that our computational modeling [47, 61-67] and machine learning work [68-73] is tightly integrated with, and drives, experimental validation [47, 74-76]. 4) Our more recent work has both revealed how to re-write (without genetic rewiring) the target morphology setpoints of regenerating organs [32, 34]. Another prior example of our pushing cellular plasticity past its genomically-encoded hardware default is the creation of novel synthetic organisms (“Xenobots”) from wild-type skin cells, with new structure and behavior despite their wild-type genome [77]. 5) The disclosed project has biomedical implications, and our work has in the past successfully targeted biomedical endpoints such as inducing limb regeneration [78, 79] and tumor normalization [57-60, 80, 81], as well as stem and other cell type manipulation in human cells [82-97]. Two biotech companies are currently investing in our work in limb regeneration and cancer normalization.

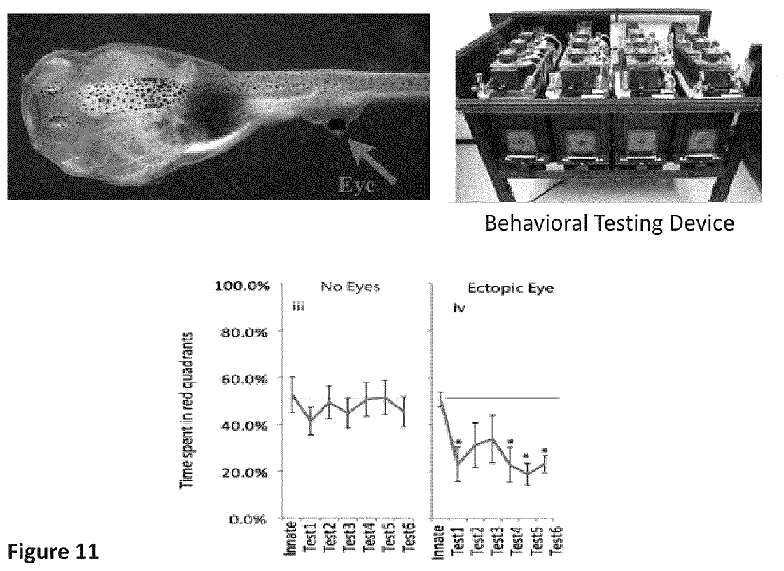

Finally, a part of our work concerns creating an integrated computer-controlled cell training device. We have significant experience in this area, having designed, built, and deployed the first automated training and testing device for planaria and tadpoles [98], which we used to study memory during brain regeneration [99] and the plasticity of vision in animals with eyes in aberrant locations [24, 25, 100] (FIG. 11). The experience gained in creating this multi-modal, real-time training platform will be useful as discussed below.

### Training Networks: Rationale

We chose the molecular pathway control problem in order to 1) to characterize a potential molecular substrate of learning, 2) show a proof-of-principle of how to exploit proto-agency for practical purposes, and 3) solve an important class of inverse problems for molecular medicine. GRNs are a paradigm case of a mechanistic framework in the biosciences (and thus, impactful if we show how basal cognition plays a role even here), and one which is facing limitations that will not be solved by big data or increasingly high-resolution (single-molecule) profiling approaches. Examples of time-dependent properties in contexts with massive unmet biomedical need are the many agonist drugs with desensitization/down-regulation potential of their receptors, anti-epileptics, chemotherapy agents, GPCR drugs, diuretics, antidepressants, neuropsychiatric drugs, etc. Once a particular network is inferred for a process of interest (e.g., neural tube development, blood pressure control, metabolism, immune system function, cancer suppression, etc.), how can we: 1) predict what events will cause long-lasting changes (disease states)? 2) reverse those states? 3) predict and reverse pharmacoresistance, where a given drug works well for a while but then ceases to be effective? 4) predict and reverse sensitization, where a given drug is well tolerated for a while but cannot be used continuously because intolerable side effects appear? 5) predict why individuals have different responses to the same therapy—to personalize and anticipate the diversity of efficacy and side effects?

Importantly, the ideal solution to these problems will not be only gene therapy: even in the very rare cases where the correct system-level outcome can be produced by making just one change in a protein structure or promoter, implementation of such changes in the many cells and tissus of a patient faces massive barriers of safety and efficacy. The ideal solution would be a judicious pulsed strategy of stimulation-using drugs (or other modalities) to trigger nodes with the appropriate timing that implements an experience to this proto-cognitive agent that causes it to learn a different behavior or motivates it with positive/negative reinforcement to a different dynamic profile (indeed, from the perspective of dynamical system theory, one way to understand learning by such networks is experience-dependent shifting into different stable attractors [22, 101], But is this possible for networks-don't networks provide static behavior that cannot be changed without physically re-wiring the connections and nodes (by altering proteins and promoter sequences)? We found that even gene regulatory networks should be trainable. Our analyses [1] help to de-risk this approach, showing that it is very likely that pathways should exhibit the hypothesized degree of plasticity and that we have the computational tools to characterize and exploit it.

### Preliminary Data on Training Networks: Computational Results

Much work has gone into computational inference of GRN models [102, 103], and the development of algorithms for predicting their dynamics over time [104]. However, the field has been largely focused on rewiring-modifying the inductive and repressive relationships between genes—to control outcome. This can be difficult to control in biomedical contexts, and even in amenable model systems, it is often unclear what aspects of the network should be altered to result in desired system-level behavior of the network. Dynamical systems approaches have made great strides in understanding how GRNs settle on specific stable states [105, 106]. However, significant knowledge gaps remain concerning temporal changes in GRN dynamics, their plasticity, and the ways in which their behavior could be controlled for specific outcomes via inputs not requiring re-wiring.

Thus, an important challenge in developmental biology, synthetic biology, and biomedicine is the identification of novel methods to control GRN dynamics without having to solve the difficult inverse problem [21] of inferring how to reach desired system-level states by manipulating individual node relationships, and without transgenes or genomic editing. A view of GRNs as a computational system, which converts activation levels of certain genes (inputs) to those of effector genes (outputs), with layers of other nodes between them, suggests an alternative strategy: to control network behavior via inputs-spatiotemporally regulated patterns of stimuli that could remodel the landscape of attractors corresponding to a system's “memory”. A broad class of systems, from molecular networks [107] to physiological networks in somatic organs [108, 109] exhibit plasticity and history-based remodeling of stable dynamical states. Could GRNs likewise exhibit history-dependence that could help understand variability of cellular responses, and be exploited to control their function by modulating the temporal sequence of inputs? This is a different approach from existing conceptions of memory as changes at the epigenetic and protein levels [110-113].

Several prior studies have suggested memory phenomena in network models [114-124]. However, there has been no systematization of the kinds of memories that such networks could possibly exhibit. We sought to rigorously define several types of memory (loosely analogous to those found in the behavioral science of neural networks), provide an algorithm with which any future network model can be evaluated for interesting memory dynamics (to make predictions for experiment), and compare existing models of important biological networks to those of random networks.

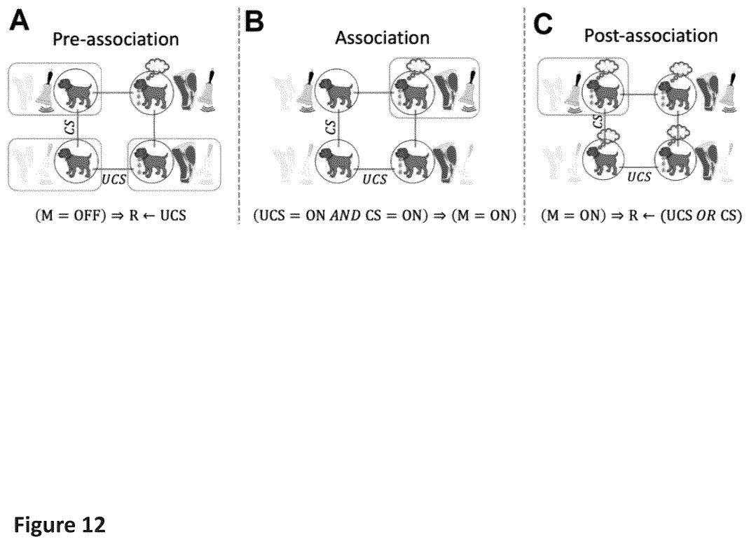

One especially intriguing possibility concerns associative learning [125, 126]. The textbook experiment by Pavlov illustrates associative learning in a specific form known as “classical conditioning” [127, 128] (FIG. 12). Initially, the dog naturally salivates when it smells food, termed the unconditioned stimulus (UCS) and does not salivate when it hears a bell ring, making the bell the neutral stimulus (NS). The smell of food and the sound of a bell are unrelated stimuli, and only one, the UCS, induces the dog's salivation (the response R). In this experiment, the dog is exposed to the UCS and NS at the same time repeatedly. Gradually, the dog learns to associate the NS with the UCS, to the point where it responds to the bell alone as if food is present, functionally transforming the NS to a Conditioned Stimulus (CS) which can now produce the response R. Although associative learning is traditionally studied as a neural phenomenon, many different types of dynamical systems can instantiate it [9, 22, 101, 129, 130]. Indeed, the original experiments of Pavlov showed associative and other kinds of learning within his dogs' organ systems [131, 132], in addition to the well-known learning of the animal via its brain.

In biomedical contexts, some drugs targeting specific network nodes are highly effective in laboratory studies but too toxic to use long-term in patients [133]. If associative memory existed in GRNs, predictive algorithms could be developed to reveal which stimuli can be used to trigger desired responses via a paired “training” paradigm. In this case, the network would associate the effects (R) of a powerful but toxic drug (UCS) with a harmless one (NS, which would become the CS). It might then be possible to treat the patient with the neutral drug (NS) to obtain the desired therapeutic response of the UCS without the side effects. This is just one example of a number of strategies that can be developed for rational control of GRN function, once the memory properties of GRNs of interest were characterized.

To achieve this, we systematized the notion of memory in dynamical models of GRNs and similar types of networks, by rigorously defining and categorizing several kinds of memory in this formalism. We then developed algorithms to analyze the plasticity of response to specific patterns of node activations over time. We first focused on a well-known class of dynamical models known as Boolean networks (BN) that was pioneered by Stuart Kauffman [134] and Rene Thomas [135] as simple coarse-grained models of GRNs. The nodes (variables) in a BN are binary, representing repression or activation. Gene states are updated over time due to interactions with other genes and their transcripts, as described by the Boolean functions associated with each node. The Boolean operators defining the relations among the genes are AND, OR, NOT, and XOR. Boolean models have proven useful in gaining dynamical insight into numerous phenomena, such as criticality [136], cell signaling [137], pattern formation and control [138], cancer reprogramming [139], drug resistance [140] and even memory in plants [141]; the Cell Collective model database [142] that we utilize in this work contains many more such published examples. For comprehensive reviews of BNs, including aspects of how they are inferred, analyzed and used to make predictions, see [143-146]. While our published work concerns Boolean GRNs, we have now extended this analysis to continuous (ordinary differential equation) models, and discovered the same phenomena (manuscript in prep.), confirming that our findings are not just a feature of the Boolean formalism.

We hypothesized that GRNs in general may be capable of diverse new kinds of memory, in that their response to future node activation events would change to implement desired network behavior, and that an algorithm could discover the necessary sequence of stimuli to make this occur predictably. Such long-term change in behavior due to experience (memory) could occur via changes at the level of the dynamical system state space, not requiring changes in inductive/repressive relationships between genes (rewiring the connectivity). We specifically hypothesized that such historicity would be an inherent property of networks but would be significantly enriched in real biological GRNs. It is important to note that the memory being tested here takes place within the lifetime of a single, constant GRN—not a process of evolutionary selection or population learning.

Long-term changes in GRNs' dynamical system states would be analogous to intrinsic plasticity in neuroscience, which functions alongside synaptic plasticity (rewiring that changes the connection weights between nodes). There is increasing biological evidence that learning and memory happen at the level of single neurons, and that memory could be stored in their dynamic activities as intrinsic plasticity due to the dynamics of bioelectric circuits [147-154]. The theoretical foundations of such plasticity-free learning have been explored [155, 156]. Thus, the existence of plasticity-free memory in GRNs would have major implications along several lines. First, it would suggest developmental programs where dynamic gene expression could result from GRNs whose functional behavior was shaped by prior biochemical interactions and not genomically hardwired. Second, it would suggest a new approach to biomedical interventions complementing gene therapy: drug strategies with temporally controlled delivery regimes could be designed to train GRNs to produce specific outcomes, shape their responses to drug and other interventions in the future, disrupt cancer cells' adaptation to therapeutics, or prevent disease states from arising in specific circumstances. Moreover, an understanding of GRNs' long-term modification by prior physiological experiences could help explain the wide divergence of drug efficacy and side effects across patients and even across clonal model systems [32].

The presence of a kind of learning in GRNs has been suggested in specific cases [105, 155, 157-162]; we performed the first systematic study of memory across diverse GRNs or analysis of possible different kinds of memories that may exist and the relationships between them. We comparatively analyzed the definitions of memory in the context of animal behavior, mapping them onto possible GRN dynamics, providing a taxonomy of learning types appropriate for GRNs and other networks like protein pathways, all without any changes to weights or mechanisms. We rigorously defined the kinds of memory that could be present in GRNs and produced an algorithm to systematically test any given GRN for the presence of different types of memory with different choices of network nodes as stimuli targets.

Analyzing a database of known GRNs from a wide range of biological taxa, we showed that surprisingly, several kinds of memory can be found, including associative memory. We also analyzed randomized versions of each biological GRN to demonstrate that the amount of memory found in a GRN is not governed solely by node number and edge density, and that real biological GRNs have more memory incidence and capacity compared to similar random networks. Comparing GRN data with analysis of randomized models revealed that the biological networks have disproportionately more memory (suggesting that biological evolution may have favored networks with memory properties, although this conclusion is in no way necessary or required for our training experiments, as they involve only real biological networks and are compatible with any degree of trainability in random networks). We also identified statistical relationships between the likelihood of a given network exhibiting a particular kind of memory and other memory types it may have, suggesting that memory types tend to occur together.

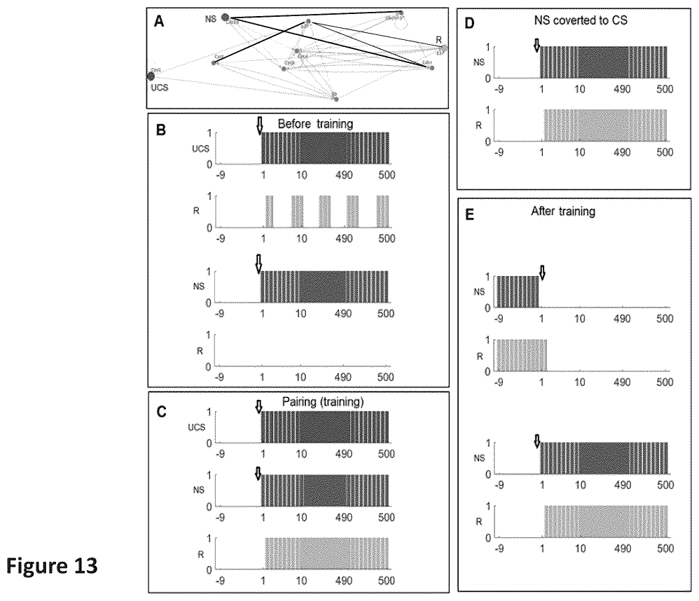

Our algorithm tests any arbitrary network model for the ability to learn in 7 distinct training paradigms. Fundamentally, each network model (A) contains a set of nodes, each of which can be up- or down-regulated by specific drug stimuli (or genetic approaches, if desired). Some of these are UCS (unconditional stimulus) nodes because their activation immediately (by default) causes a Response (up- or down-regulation of some node of biomedical significance—a specific protein, or metabolic state, or physiological readout). Other nodes are Neutral at first (do not have any effect on Response), but are candidate CS (conditioned stimuli)—our algorithm tries training the model using various timed application of stimuli on the different nodes to discover which can be efficiently used to change how the system responds to inputs in the future (FIG. 13). We predicted that evolution would have already exploited proto-cognitive functions in pathways because these would have been necessary for the observed degree of plasticity, robustness, and reprogrammability we observe in developmental genetics contexts [7-9, 19, 20, 22, 38, 101]. In other words, our conceptual framework for unconventional intelligence made specific predictions which we computationally confirmed using existing (published) network models, and now will be validated at the bench.

Our computational analysis [1] showed two important things. 1) Biological networks are predicted to have remarkable trainability in several different paradigms (some of which map cleanly onto known training techniques in behaviorist and cognitivist theory, and some of which are novel and unique to network models as far as is known to date). 2) Random networks show much less of a capacity for learning, consistent with our hypothesis that evolution (either directly or indirectly) favors this property and has selected for it. Basically, our approach is to treat the nodes in such networks as stimuli or response elements. Stimulating a node means providing an up- or down-regulating influence (e.g., a pulse of a drug that activates a specific protein). The response will be anything that we seek to predict and control (e.g., cell migration, metabolic state, etc.). Our goal was test a given model to answer this question: which nodes should we stimulate, in what pattern, to induce desired behavior (mapping of inputs to response output) in the future?

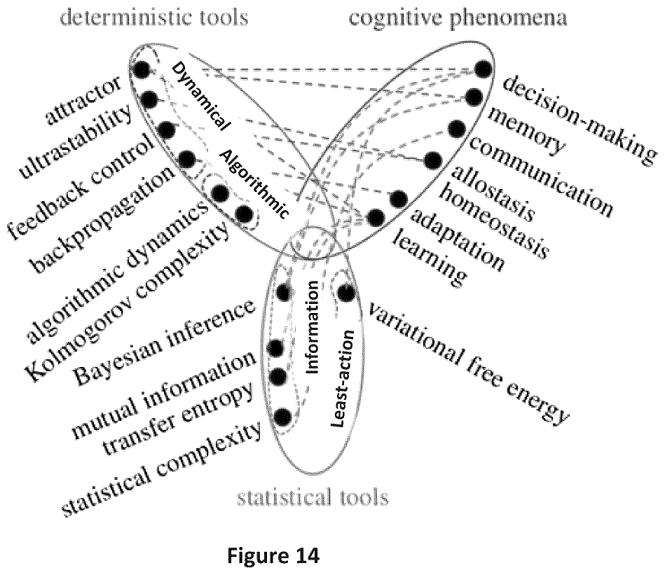

The current state of affairs is as follows. Molecular medicine and developmental genetics have made the most progress with pathway steady-state outcomes, not time-dependent behavior. Biomedicine treatments target symptoms, which reappear when the drug is withdrawn because the system has not fundamentally been shifted into a new state, only low-level response elements temporarily silenced. Many drugs stop working after initial efficacy (pharmacoresistance), some become intolerable over time (sensitization), and in general it is very difficult to predict which will work and which will fail (and for which patients). Computational properties of GRNs and pathways are not well-understood either in development or in disease/physiology contexts. We have will examine unification of several ways to see complex systems, as each has specific advantages for their control [22] (FIG. 14). We created and published the formalism and a v1.0 of software for detecting learning in molecular networks [1]. The next key step is to validate this approach (achieve improved prediction and control) at the bench, in a biomedically-relevant context by using a training paradigm. Success would draw a vast community of academics and workers in pharmaceutical R&D into new and productive collaborations, unleashing new resources, and establishing the foundation for new biomedical interventions.

### Research Plan

Our action plan is focused around the following key steps, which are necessary to establish a platform that will facilitate learning-based intervention discovery for various areas of biomedicine and give insight on how basal components of cognition arise in the humblest origins of biology: (1) Build a device for high-throughput, multiplexed computer controlled systems to provide any desired stimulus rhythm for cells and simultaneously monitor their response. (2) Improve the memory-detection software, and develop machine learning algorithms to help shape optimal training paradigms based on real-time cell response data. (3) Screen a variety of training types, drugs, and cell targets to test our hypothesis and establish both its ideal early applications and its likely limitations. (4) Narrow down to a clinically-relevant example and show unequivocally that this approach works.

### Section (1) Building a Memory Screening Platform for Cells and Tissues

Goal: Our major goal in this project is to demonstrate training of molecular networks in real living cells. Thus, the first step is the production of a platform, to be disseminated widely in the academic community and industry, that facilitates the identification of drug treatment regimes (time-dependent experiences) in arbitrary cells or tissues that induce the desired outcomes. The goal is not merely to answer a specific narrow scientific question but to catalyze the process of discovery of novel applications of these ideas by providing a system to the community. This “robot scientist” [163-166] will enable high-throughput experiments in which it stimulates the sample and records responses, looking for signs of learning. It will test and refine specific training protocols for a chosen biological sample and a pathway of interest. Specifically, it will perform behavior shaping experiments, varying hyperparameters (like duration and amount of stimulus, training regime, etc.) and use machine learning to refine (parametrize) a model of the relevant regulatory pathway. Unlike the complementary approach discussed below, this system does not need a good model of the pathway as input, and thus is applicable to numerous cases where a fully specified network model is not available.

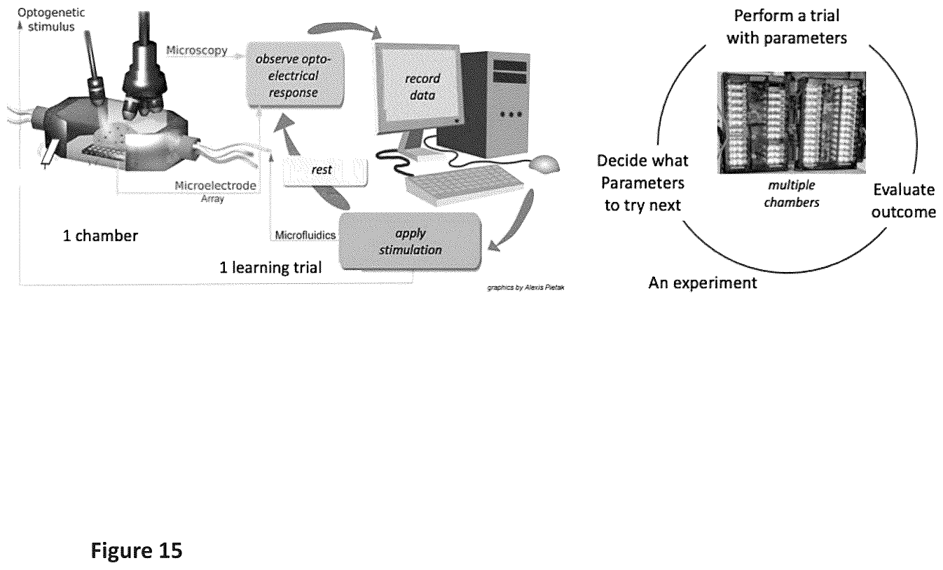

Approach: The logic of the platform is as follows. One biological replicate consists of an environment (e.g., a petri dish) with a Sample of cells to be trained. One training Session consists of a several-day trial during which the sample is continuously stimulated by pulsed drug treatments (delivered by a mesofluidic mechanism such as the one we have already created at Wyss institute) and the response monitored by fluorescent and/or biochemical sampling. The data encompassing how response of the network to the drug stimuli at the end of the Session differs from that at the beginning of the Session (degree of training achieved) are fed to the machine learning component. These Sessions occur in parallel in a multiplexed fashion (96- or 24-well plates, depending on cell or tissue context) in parallel, which provides biological and technical replicates for robust learning assessment. An Experiment consists of a sequence of Sessions, each performed with a new set of samples, as the machine learning component alters the design of the stimulation to try in each Session to improve the training based on the past Sessions' data. The full device will be built by end of year 2, but in the first year, we will build a manual version of this that will be sufficient to perform trials with known pathways as discussed below while the high-throughput and machine learning components are being worked out. A single such chamber, the control loop for a single Session, and the control loop for a whole Experiment (C) are shown in FIG. 15:

We have begun establishing an engineering specification for the device (for example that needs to exchange up to 2 ml of media in under 30 seconds in gentle, distributed flow that doesn't disturb the cells), the optical imaging system, and the real-time control response specifications (running on a fast CPU with a real-time operating system). We believe the relevant time scale for cellular pathways to be in the minutes scale, but will engineer the system to go down to 100 millisecond resolution in case it becomes necessary.

A typical experiment looking for associative learning might be performed as follows. First a Session is performed to check that neutral drug application does not trigger the response. Then a Session is performed to check that the potent drug indeed triggers the response. Then an experiment containing many Sessions is performed, and in each session: the system stimulates the cells with paired in-flows with potent and neutral drug for some number of exposures, then stops the potent drug and only stimulates with the neutral drug. It observes whether the association has been formed (whether the neutral drug alone causes the response), records the information, and repeats the Session using different choices of neutral drug, and different values for concentration of each drug, dosing pulse width, rest, number of pulses, etc. searching for the optimal combinations. The Response is checked optically (for fluorescent readouts such as tagged proteins or cell shape/number), electrically (using the microarray), or chemically (using reporter electrodes). Another typical experiment addressing pharmacoresistance would measure response over time during repeat stimulations by a drug, and then ask what kind of stimulation regimes most potently avoids the habituation.

Typical experiments will include for example: (1) associating oxphos inhibitors with very low dose aspirin or routine supplement compounds, (2) associating steroids with very low dose aspirin or routine supplement compounds, (3) abrogating pharmacoresistance for a typical anti-epileptic, 4) abrogating pharmacoresistance for nicotine, 5) abrogating sensitization, and others. The specific choice of drugs will be made in concert with our pharmaceutical partners for optimal impact on human patients, because once operational, this system will be able to test a wide variety of drugs for many indications against diverse types of cells and organ culture.

The machine learning component, operating as a Boltzmann machine as we used previously to fit functional serotonergic network data [167], will parameterize an internal model of any given pathway so as to optimally fit the observed stimulus-response data; this model will then be used to design progressively more-efficient interventions, as we did in [1] using human-generated models.

The major hypothesis to be tested in this section is that a suitable automated stimulation and recording platform, together with a machine learning core, can identify parameters for a successful training regime that demonstrates associative conditioning, abrogation of pharmacoresistance, and abrogation of sensitization. Success will be determined by the same kind of association, habituation, and sensitization curves of stimuli vs. response that are familiar to workers in classic behavior science.

Potential difficulties and their mitigation: The device will be built in modules (culture chambers, computer-controlled drug wash-in/wash-out, computerized live imaging and electrode measurements, real-time process control), by one of several possible machine shops. We will discuss in detail and get quotes from Boston Engineering (who worked with us on the automated training device) and Draper Labs. Together with the electrical, chemical, and optical engineering expertise available in our group and the state-of-the-art facilities at the Wyss Institute, we will be able to build and integrate a working, real-time device for treating cells with drugs on a timed regime and observing the effects via optical, electrical, and chemical readouts. We have extensive experience debugging and modifying such integrated systems. None of the individual components require new science-only established system integration techniques.

The machine learning will be done in-house, as we have several high-level experts in the Levin lab. We will choose tools and approaches that are compatible with the overall volume of data we will gather. For example, if the Boltzmann machine does not perform well, we can readily shift to a neural network approach [71, 77, 168-171] or evolutionary computation, in which we have a track record of experience.

### Section (2) Create Computational Methods for Predicting Memory and its Control in GRNs

Goal: While the section above discusses the creation of a device for unbiased training of cells and tissues that does not require any knowledge of specific pathways, this section seeks to improve our software for analysis of memory in existing models. Many models are known of networks governing important pathways, and many more are being discovered, reconstructed, and published all the time. Here, we will produce new theory showing how dynamical systems models and memory models can be simultaneously valid, complementary descriptions of a multi-scale problem of prediction and control. We will improve capabilities of the software, which will result in a powerful tool that everyone in the community will be able to use to predict different types of memory behavior in their networks of interest, and test-drive candidate stimulation protocols in silico to predict what kind of regimes would be ideal for improving functionality while reducing undesirable aspects of treatment with the usual single, constant dosing paradigm.

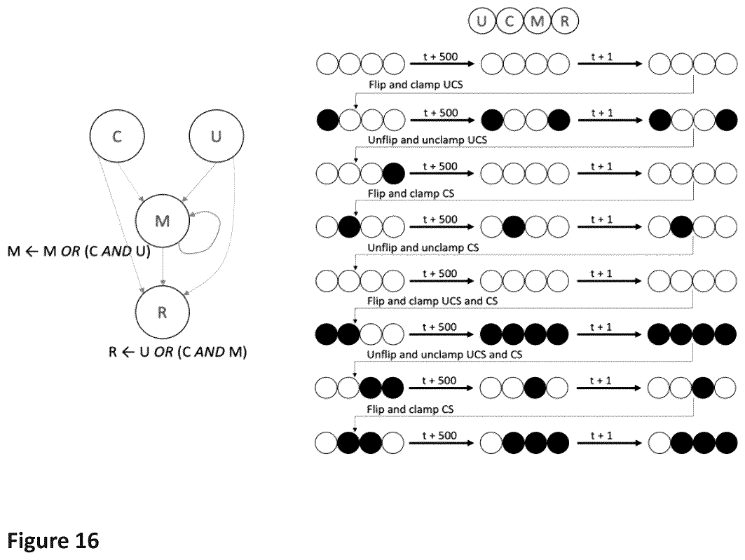

Approach: It may be helpful to first show an example, using a very minimal sample network, of how a kind of memory (in this case, associative) is tested by our algorithm (FIG. 16). The detection of associate memory (AM) in a Boolean model follows five steps: A) initialization; B) verifying that the UCS alone is able to trigger R; C) verifying that the neutral stimulus alone is unable to trigger R; D) conditioning the neutral stimulus; and finally E) verifying that the CS is able to trigger R post-conditioning. These steps are described in detail below. A) In the initialization step, the network is started with a state of all zeros and synchronously updated for 500 steps. At the end of the updates, this network settles on a fixed point state of all-zeros states (indicated by this state repeating itself after one step following the end of the previous updates). B) In this step, the final state from step (A) is taken and the UCS is flipped and clamped in that state. At the end of 500 update steps, this network settles on a fixed-point state with R flipped. The network is then relaxed, where the UCS is restored to the original (unflipped) state, is unclamped and then updated for 500 steps. This network enters a fixed-point state at the end of the updates. C) In this step, the final state following relaxation is taken and the CS is flipped and clamped in that state. At the end of 500 update steps, this network settles on a fixed-point state with R remaining in the same original state (not flipped). The network is then relaxed, where the CS is restored to the original (unflipped) state, is unclamped and then updated for 500 steps. This network enters a fixed-point state at the end of the updates. D) In this step, the final state following relaxation is taken and both the UCS and CS are flipped and clamped in that state. At the end of 500 update steps, this network settles on a fixed-point state with R flipped. The network is then relaxed, where the UCS and CS are restored to their original (unflipped) states, unclamped and then updated for 500 steps. This network enters a fixed-point state at the end of the updates. E) In this final step, the final state following relaxation is taken and the CS is flipped and clamped in that state. At the end of 500 update steps, this network settles on a fixed-point state with R flipped, thus showing that the previously neutral CS is now conditioned to trigger R.

We will next improve the software as follows. The existing code is able to analyze Boolean models for 7 types of memory. The next functionality to implement: 1) fully extend the modeling of training to ODE (continuous, ordinary differential equation) models, 2) extend from single-cell networks to coupled networks modeling a multi-cellular tissue, 3) extend those models to once encompassing functional microbiome interactions (i.e., allow interactions of distinct networks belonging to host and symbiont/parasite, as some behaviors may be a function of the bacteria, or synergistic interactions with the patient's cells), and most powerfully of all, 4) a generalized evolutionary search system which can discover treatment regimes to induce as close as possible any desired behavior in a network. We will also produce 5) a comprehensive graphic user interface (GUI) that will make easy for users to specify one of many training paradigms to look for, 6) a flexible pattern-description language (akin to REGEXP) that would enable users to specify what pathway behavior they are seeking to induce or counteract, and 7) a visualization system that illustrates the principal components of the state space of the network in order to reveal the most powerful kinds of memories and how the network can be made to attain them.

The major hypotheses to be tested here are that a software strategy can be defined which takes a known network (GRN, protein, or metabolic) specification (via the standard Systems Biology Markup Language) as input, and outputs a full profiling of the kinds of memories of which it is capable, including the stimulation regimes for each one (which nodes and how strongly, how often, and with what timing). A subsidiary hypothesis is that this can be generalized to a search process in which a stimulation sequence is identified that can abrogate habituation or sensitization (if such exists), and to make other predictions like deterministic chaos (extreme sensitivity to initial conditions), map out the possible memories in a dynamical systems state space portrait, etc. We will specifically include models of association of drugs in place conditioning [172, 173] and learned association [174], and pharmacoresistance in circuits involving GPCR drugs, antiepileptics, SSRIs, and cancer chemotherapies [175-177]. Especially good examples are likely to come from the ERK signaling pathway, where excellent prior work has identified numerous feedback loops and modeled them in significant quantitative detail [178, 179].

Another important effort in this section is to incorporate our models with existing work on dynamical systems approaches [180, 181]. The existing methods for evaluating the reachability of attractors, such as developed in [182, 183] will be added to the software. We will also produce a visualization module that can reveal the existing possible memories in networks as attractors in their state space, and reveal how training stimuli can shift the system among such stable states (as well as how the different kinds of memories we identified map onto changes in dynamical system paths). We will pay special attention to limit cycles, and develop further the mapping we begun in [22] between specific concepts in dynamical systems theory and those in learning. In particular, it will be critical to extend our framework to include periodicity (limit cycles)—the theory will have to be extended to include a rigorous definition of node state over time that may not be flat but could be cycling or even meandering around a specific trajectory. The dynamical systems approaches will be important here, but we will also take advantage of work in the neuroscience of learning circuits, which likewise are not static but can represent memories as recurrent loops of activity. The variability as a function of time could be best treated as noise (coarse-grained away) or as stochastic elements which actually help the function of the system (e.g., stochastic resonance-like effects). All of these tools will be integrated toward control of outcome.

While our current software from [1] simulates dynamical learning exclusively, the new version developed in this section will be more inclusive, enabling scenarios in which receptor modification and other chromatin epigenetic or post-translational events work together with the dynamical system memory to enable learning in mixed scenarios of “synaptic plasticity” between the node links and true dynamical system memories. This will enable application to a wider set of cases in the sections discussed below. For example, we may find that even examples of pharmacoresistance that involve wiring changes, such as down-regulation of their receptors (traditional plasticity, not dynamical memories) [175-177] can be reversed by interventions that are only training regimes and not themselves require rewiring. In this section, we will attempt to find such interventions using exploration of models of habituation and sensitization integrated with our training simulator. We will likely also incorporate information theory metrics, which have been proven useful in understanding desensitization of receptors [184], as the most powerful insights would result from unifying three perspectives (information theory, learning, and dynamical systems theory) on the same phenomenon.

We also will perform an in-depth study (via simulation and statistical analysis) of what factors are predicted to control the duration of memories, the tendency of certain memory types to co-occur, the specificity achievable by new instructive associations, and the effects of cross-talk among different networks. This will be done in the current suite of Boolean and ODE models and compared with results in a much larger set of randomized models as controls.

Potential difficulties and their mitigation: The results of the software applied to real networks will be utilized in other experiments disclosed herein, to help guide the choice of cell type and drug targets. We anticipate no difficulties in capabilities 1-3,5-7 as described above, as they do not involve many unknowns. There are decisions to be made (for example, how to handle network nodes that are cycling, not static, and how to quantify the effect of drug interventions as up- and down-regulation of target nodes, but these are already being worked on now and can always be adapted on-the-fly as data appear. Item #4 is the most open-ended one; if the genetic algorithm approach turns out not to be efficient at identifying stimulus regimes for achieving specific behaviors (e.g., preventing habituation), we will turn to an artificial neural network adversarial approach [185], where an ANN is rewarded for the ability to predict and manipulate a target regulatory GRN (an in silico simulation of real world evolution where parasites and commensals evolve to gain control over hosts by hijacking their physiological networks, as we have described between bacteria and planarian regeneration [186, 187].

### Section (3) Screen Cells, Drugs, and Memory Types to Map Out the Terra Incognita of Cognition in GRNs

Goal: Here, we will deploy the system discussed in section (1) (first manually, and then in an automated, high-throughput parallelized fashion), together with the guidance of the software from section (2) (applied to the most relevant networks) to characterize examples of associative conditioning in gene-regulatory networks, and show examples of using computationally-derived stimulus regimes to prevent pharmacoresistance and sensitization. The goal is to identify potentially clinically-relevant cases to serve as our flagship examples of this approach. We will also be looking for additional features of primitive cognition in these cells, including the ability to anticipate timed stimuli [188-193], endogenous rhythms [194-199], etc. The goal of this section is a broad survey of cell types and pathways, to understand how widespread the different types of learning are. We will search here beyond GRNs, to analyze examples of protein and metabolic networks.

Approach: The system will be tested with a variety of human cell lines (including epithelial cells, macrophages, iPSc-derived neurons, pancreatic beta cells, and tumor cell lines), and neural and non-neural organoids as we have published on in the past [82-87, 95, 96]. Initial examples include searching for ways to prevent or reverse habituation (pharmacoresistance) to chemotherapy drugs in acquired androgen resistance syndrome in testicular tumor lines, sensitization to corticosteroids in epithelial cells, anticipation of sugar pulsing in pancreatic cells, and functional association of the powerful drug Rapamycin to low-level aspirin.

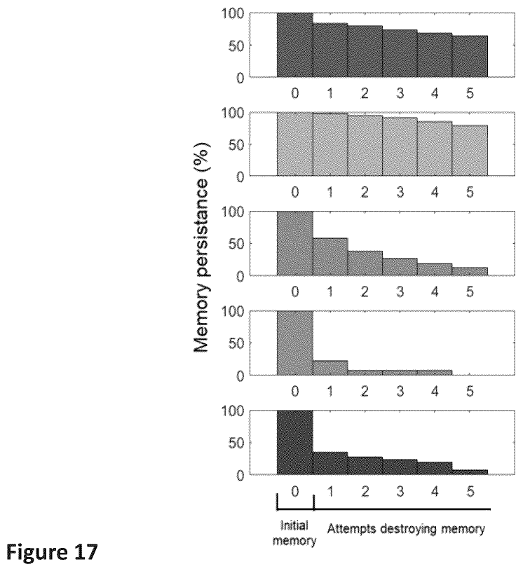

We will make a priority list for biological targets and cell types, to be used in section (1) and also to be the most important networks to analyze with the new code in section (2). An example of the kind of data we expect, in this case coming from an analysis of ODE (continuous) models for breaking pharmacoresistance (labeled as memory) in the graph is shown in FIG. 17—each “attempt” is a stimulus that has been predicted to abolish the memory, and this graph shows the predicted successful and unsuccessful cases (the network represented by yellow bars in the 2^{nd} row, and to some extent the network shown in the first row as blue bars).

We will first use human-designed training regimes guided by software analysis using our existing code and knowledge of published pathways for cancer, drug addiction, and immune system function. In each case, we first validate candidate nodes as UCS (able to cause response R), and NS (not causing response R when triggered, even after a UCS has been seen). We take measurements of all 3 nodes as a function of time, to note any unexpected effects or limit cycles (periodic behavior), and use those to revise the model or the time scale at which we are simulating. As the new code (section (2)) and the screening platform device (section (1)) come online, we will increasingly rely on them to guide experiments. We will initially search broadly through several areas and then drill down as soon as we find a couple of promising examples. We will also include an example of microbiome, such as interaction of pathogenic and non-pathogenic E. coli [200, 201] with human neural organoids and macrophages [83, 202-204], to train the combined system for improved tolerance and cooperation using inflammation markers as readouts. Success will consist of identifying two different, highly reproducible examples of association, reduction of habituation, and reduction of sensitization.

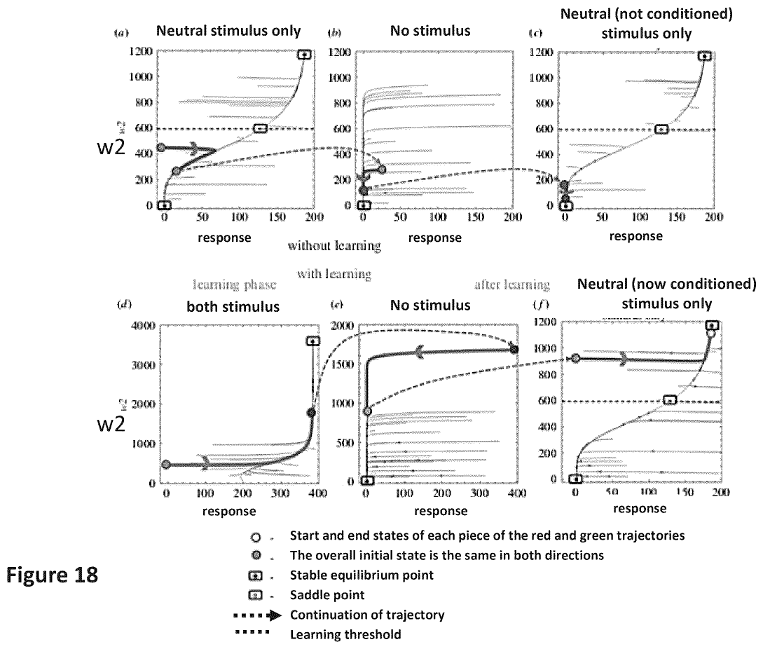

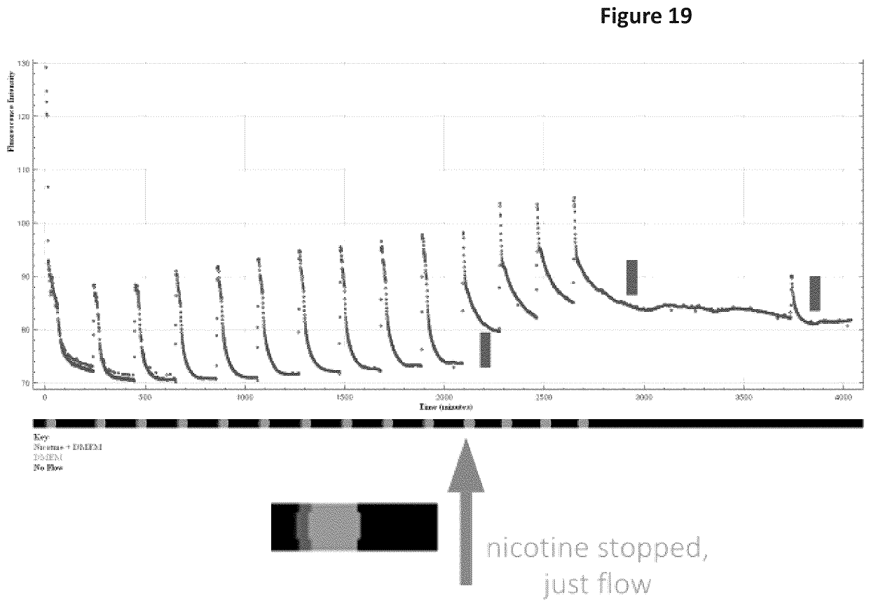

For each of these systems, we will attempt to illustrate the effects we observe as both instances of learning and as dynamical systems portraits, to get a solid characterization of the success of each approach in predicting the capabilities of each model. Examples (from toy model networks) are shown in FIG. 18, to indicate the kind of insight that can come from this effort.

Potential difficulties and their mitigation: Trouble-shooting of the device, and debugging of the software, will be accomplished in section (1) and section (2) respectively. There is expected to be some work to optimize the growth of the various cell lines and titering of the drugs in each system, but this is relatively straightforward and we have many resources in the Harvard Medical School area for almost any conceivable cell model system. Another possible barrier will be the fact that some Response nodes of interest will not have a convenient electrophysiological, biochemical, or other readout. In this case, we will engineer the cells by producing a fluorescent fusion protein or a fluorescent sensor that reports the status of the response node (or use something like the FUCCI proliferation system, if the output of interest is a cell-level phenotype such as mitosis).

If it turns out that association, or management of pharmacoresistance or sensitization are hard to achieve, we will test other combinations of drugs for CS, UCS, and R, as well as cell types. While the question of whether these learning phenomena exist in cells is the central issue to be answered in this project, and thus represents a major unknown to be learned via this work, we believe (based on our published analyses of network models and the many examples of cellular plasticity we cite above) that it's very unlikely that given the many available options for drugs and cells that we will not be able to identify good examples. One other key finding bears on this question. We reported [1] that biological networks have much more well-developed memories than similar randomized models. Thus, it appears that either directly or indirectly, evolution favors trainability in its networks, which helps lower the risk inherent in looking for memory [155, 205-207] in living cells.

Section (4) Present the Biopharma Community with a Clinically-Relevant “Killer App”

Goal: The goal in this final section will be to narrow down the broad survey of section (3) into a powerful, well-characterized set of examples of how learning in networks can be exploited for a clinically-relevant context. The most impactful outcome would be the equivalent of a “killer app” in computer science-one that is so compelling in achieving a novel capability that it focuses attention of the community to adopt the approach, software, and screening device. Clinical data are already beginning to give support for our hypothesis, showing that only specific nodes work well as stimuli with which to modulate long-term properties of pathways (which our work will identify) [174, 208-210].

Approach: We will prioritize examples (from section (3)) of memory that are the strongest, most reproducible, and most relevant, characterizing them in great detail to identify a set of drugs, pulsing regimes, and outcomes that would be usable in human patients. It may turn out to be associative learning, but may also be better as examples of breaking pharmacoresistance (in the case of cancer cells adapting to chemotherapeutic agents for example). We will, in consultation with our industry partners (e.g, Juvenescence, who is currently funding our limb regeneration efforts), obtain the necessary dataset that would enable us, at the end of this project, to form a commercial partnership that would pay for large-scale in vivo (mammalian animal system, likely rabbits) preclinical testing and ultimately a clinical trial. The details will necessarily be worked out along the way, but we will follow well-established roadmaps to go from computational prediction and in vitro data to preclinical and then clinical validation. Excellent candidates are likely from among GPCR drugs, antiepileptics, SSRIs, and cancer chemotherapeutics, and pathways like ERK and Wnt signaling [211, 212].

We will characterize important aspects including: 1) what is the right time scale for training—how often does the patient need to change drug state and is it compatible with the metabolics of the drug, 2) what is the duration of the training-how long does the memory last before needing to be re-trained, 3) what side-effects can be expected with a given implementation. All of this will be predicted based on the in vitro work, and we will set up the roadmap toward pre-clinical testing (in rodent or similar models) with our collaborators (such testing is part of the next steps after this grant is completed, and by then should be very fundable by NIH or disease foundations).

Rather than picking a definitive list of drugs (and networks to train) in advance, our project plan for this section includes an explicit consultation kickoff meeting, where we will consult with members of our main bio-pharma partner (Juvenescence LTD, who is funding our limb regeneration startup company, and Takeda Inc.), members of our Allen Center advisory board with clinical and biomedical expertise (Callum MacRae), members of the Wyss Institute senior leadership (Donald Ingber, Angelika Fretzen), and others. We plan a specific kickoff event where a group of about 10-15 leaders from relevant areas of biology and medicine will meet with our team to discuss and guide choice of initial cell types, drug stimulus, and response options that are the most relevant to patients and also represent the most likely to contain examples of learning from stimulus experience.

Specific success will include the demonstration of a biomedically relevant pathway and drug treatment regime which achieves a level of control over system behavior that was not previously demonstrated. The highest level of success will involve drug repurposing, which will have the greatest impact-showing how to use a drug that is already human-approved in a new indication, by software-guided timed applications. Also successful would be examples of how to use drugs in pulsing regimes that “failed” in traditional, constant-dose use. Another important part of this phase will be licensing the design of the screening device to a company (such as Fisher or Perkin-Elmer) so that large-scale production can make it available to all researchers world-wide.

Potential difficulties and their mitigation: Here, we will examine biopharma as an additional step over and above bench-work academic proof. There are many considerations that come into play, including effects on nodes other than the node of interest, potential toxicity, etc. All of this will be decided together with our molecular medicine collaborators (see letters of support) to identify the most likely flagship applications, once the data of sections (1), (2), and (3) show us the best low-hanging fruit.

A potential difficulty would be if the most effective timing was on the scale of minutes, which would be difficult to achieve in human patients by traditional dosing. If use cases justify it, we would work with biotech (including contacts at Wyss) on applications involving implantable drug pumps, which are an available technology for implementing more rapid pulsing protocols. Another question is the final cost of the screening device to end-users; we can't know this yet, but given the cost of confocal microscopes and cell screening robots that are widely used, we don't believe it will be unattainable to most labs. Central facilities would also will be able to run searches for high-impact outcomes as a service.

### Rigor, Reproducibility

Our project will be performed in accordance with the best practices of research as defined for example by NIH policies. All work will be rigorously analyzed by a professional statistician (who will also have input at the very beginning into experimental protocol design).

### Commitment to Open Science

Our group has a commitment to democratizing and making transparent the scientific enterprise, for example via the “Science at Home” initiative that PI Levin is spearheading (see for example www.the-scientist.com/news-opinion/opinion-use-the-pandemic-to-expand-the-lab-to-the-home-67677). The protocols, schematics, and data arising from this work will be published in open access journals, and provided to the community via preprints whenever possible (we have been moving toward open preprints; see examples of some of our preprints here: ase.tufts.edu/biology/labs/levin/publications/preprints.htm). Software will be disseminated via our website (as we already do with our other software: ase.tufts.edu/biology/labs/levin/resources/software.htm), and molecular reagents will be made available via repositories such as Addgene. Whenever practical, we will pre-register clear milestones and hypotheses on platforms such as OSF (and we are open to discussions of other pre-registration models).

### CONCLUSION

Numerous problems in biomedicine and fundamental life sciences face the inverse problem that affects all complex emergent systems: how do we control system-level behaviors by manipulating individual components? This problem is as salient for bioengineers and clinicians seeking to regulate gene expression cascades as for evolutionary developmental biologists seeking to understand how living systems efficiently regulate themselves to ensure adaptive robustness [213, 214]. An important direction in this field is the discovery of strategies that exploit patterns of input (experiences), rather than hardware rewiring, to achieve desired changes in network behavior or explain the modification of pathway properties faster than occurs during evolution. This requires the development of algorithms to identify specific patterns of stimuli that exert stable, long-term changes in behavior, thus characterizing endogenous memory properties of the system. Such insight would shed important light on evolution and the mapping between the Genome and the highly functional forms that function in a complex world.

Showing that we can train molecular pathways would take advantage of existing computational capabilities of the system and effectively offload much of the computational complexity inherent in trying to manage GRN function from the bottom up. Such approaches [19], if the GRN structures were amenable to them, would enable the experimenter, clinician, and indeed the biological system itself to reap the same benefits as training provides for neural systems. This approach was motivated by the advances of neuroscience, which reveal how nervous systems and artificial neural networks learn from experience. Recent advances in the field of basal cognition (memory in aneural and pre-neural organisms [9]) have revealed a broad class of systems, from molecular networks [107] to physiological networks in somatic organs [108, 109], that exhibit plasticity and history-based remodeling. Based on the remarkable flexibility observed at the anatomical and physiological levels [25, 215-219], and the conceptual similarity between GRNs and neural networks [114, 220, 221], we established a formalization of memory types for GRNs and implemented a suite of computational tests that revealed trainability in a range of biological GRNs. Importantly, evolution discovered this very early, as even unicellular networks (such as gut microbiome networks) were analyzed to have larger memory capabilities than random networks.

Our work will advance both, the science of molecular control and the understanding of basal intelligence, via a practical synthesis. We will achieve this through a combination of computational modeling, automation, and real-time cellular physiology. Our work includes a broad survey of cellular learning capacities, and a drill-down to a few flagship examples which will galvanize a merger between the basal cognition and the genetics/biomedical communities, to the great benefit of both.

### Theory of Change

We seek answers to questions that have occupied many profound thinkers in the past: how to solve the inverse problem for complex networks (and how does evolution solve it, for internal control of cell networks by other endogenous cell networks), and how to understand the basic properties of learning mechanisms that rely on physical plasticity or dynamical systems properties. We will leverage the rich history of work on these profound topics, advancing the state of the art with a new, experiment-focused approach.

The dominant paradigm in the study of GRNs by academics has been that of dynamical systems theory. There will be a tendency of the field to see our results entirely from that perspective. Thus, our publications (and the analysis of section (2)) will make very clear how the existing work in dynamical systems can be used to infer and characterize possible network memories as attractors, but at the same time how protocols rooted in training methods facilitate the identification of stimuli that shift the system into desirable regimes. One of the members of our team has extensive experience applying dynamical systems theory tools to cognitive questions (e.g., [222, 223]), and he and I have already published both conceptual and novel computational work discussing the relationship between those two approaches [22, 101]. It is important to note that despite decades of dynamical systems analysis of gene networks, no one has yet demonstrated training of these systems in the way that we suggest. Thus, it is clear that approaching these problems from the perspective of learning is suggesting novel, testable hypotheses. One limitation of the dynamical systems approach is that it requires one to have a complete, parametrized knowledge of the network (which is often not possible with relevant biological systems—there are simply too many real parameters to be practical to measure; and even then, its complexity may preclude a full analysis). In contrast, the proto-cognitive paradigm (and our disclosed device) will enable workers to screen and test out training regimes in their favorite cells and pathways without needing complete information (much as we can train animals without a full understanding of neuroscience). The power of our approach is that it searches a simpler (large-scale, behavioral) space which is exactly the most difficult part for the microphysicalist approach to reach. Our goal is to unify these two approaches, showing the relative advantages of each, and demonstrating to the biotech community how they can be practically used.

It is also important to point out that we approach this problem with significant humility, recognizing its complex nature and the possibility that we may turn out to be wrong about important aspects, which will cause us to revise the approach and the theory. While we are optimistic and excited about the potential for progress, we are not naïve about the many difficulties, both conceptual and practical, that will have to be overcome. This realistic appraisal drove the design of the budget-such an effort cannot realistically be achieved by small-scale incremental work. The TWCF's unique commitment to novel, interdisciplinary ideas is going to be necessary to enable all of the activity that together will advance understanding.

One potentially challenging area will be the linkage between the theoretical and the empirical work. It is an advantage of our approach that we will be able to try training even those pathways for which we do not yet have a good analysis (the device in section (1) will have the throughput to enable testing of a significant number of likely candidates). Thus, in the absolute worst case, what we should end up with is a set of empirical advances in real cells and a good theoretical understanding of very simplified network cases. In the optimal case, we will be able to do both in the same networks.

Importantly however, we are not going to be purists about dynamical memories. While our recent paper [1] studied the case of only dynamical memory (no plasticity at the hardware level) to show that it is possible, our real models (section (2)) will certainly also be developed to include scenarios in which receptor modification and other post-translational events work together with the dynamical system memory to enable learning.

Future work will also focus on developing a better understanding of the processes that drive networks to develop this capacity, integrating our models into evolutionary simulations of physiological and anatomical control mechanisms. We will, in particular, be interested in the forces that promote or suppress learning plasticity in regulatory pathways, and how these can be exploited by an organism's own subsystems for evolvability and robustness (e.g., regeneration) as well as by conspecifics, parasites, and collective (hive) dynamics.

### References for Example 2

[1] Biswas, S., Manicka, S., Hoel, E. & Levin, M. 2021 Gene Regulatory Networks Exhibit Several Kinds of Memory: Quantification of Memory in Biological and Random Transcriptional Networks. iScience, 102131. (DOI:doi.org/10.1016/j.isci.2021.102131).
[2] Epstein, R. 1984 The Principle of Parsimony and Some Applications in Psychology. J. Mind Behav. 5, 119-130.
[3] Morgan, C. L. 1903 Other minds than ours. In An Introduction to Comparative Psychology (ed. W. Scott), pp. 59-.
[4] Levin, M., Keijzer, F., Lyon, P. & Arendt, D. 2021 Uncovering cognitive similarities and differences, conservation and innovation. Philos Trans R Soc Lond B Biol Sci 376, 20200458. (DOI:10.1098/rstb.2020.0458).
[5] Lyon, P., Keijzer, F., Arendt, D. & Levin, M. 2021 Reframing cognition: getting down to biological basics. Philos Trans R Soc Lond B Biol Sci 376, 20190750. (DOI:10.1098/rstb.2019.0750).
[6] Qadri, M. A. & Cook, R. G. 2017 Pigeons and humans use action and pose information to categorize complex human behaviors. Vision Res. 131, 16-25. (DOI:10.1016/j.visres.2016.09.011).
[7] Levin, M. 2020 Life, death, and self: Fundamental questions of primitive cognition viewed through the lens of body plasticity and synthetic organisms. Biochemical and Biophysical Research Communications. (DOI: doi.org/10.1016/j.bbrc.2020.10.077).
[8] Levin, M. 2019 The Computational Boundary of a “Self”: Developmental Bioelectricity Drives Multicellularity and Scale-Free Cognition. Front Psychol 10. (DOI:10.3389/fpsyg.2019.02688).
[9] Baluška, F. & Levin, M. 2016 On Having No Head: Cognition throughout Biological Systems. Front Psychol 7, 902. (DOI:10.3389/fpsyg.2016.00902).
[10] Mar, R. A., Kelley, W. M., Heatherton, T. F. & Macrae, C. N. 2007 Detecting agency from the biological motion of veridical vs animated agents. Soc Cogn Affect Neurosci 2, 199-205. (DOI:10.1093/scan/nsm011).
[11] Dennett, D. 1987 The intentional stance. Cambridge, Mass, MIT Press.
[12] Alvarez-Buylla, E. R., Balleza, E., Benitez, M., Espinosa-Soto, C. & Padilla-Longoria, P. 2008 Gene regulatory network models: a dynamic and integrative approach to development. SEB Exp Biol Ser 61, 113-139.
[13] Huang, S., Eichler, G., Bar-Yam, Y. & Ingber, D. E. 2005 Cell fates as high-dimensional attractor states of a complex gene regulatory network. Phys Rev Lett 94, 128701.
[14] Peter, I. S. & Davidson, E. H. 2011 Evolution of gene regulatory networks controlling body plan development. Cell 144, 970-985. (DOI:10.1016/j.cell.2011.02.017).
[15] Davidson, E. H. 2010 Emerging properties of animal gene regulatory networks. Nature 468, 911-920. (DOI:10.1038/nature09645).
[16] Singh, A. J., Ramsey, S. A., Filtz, T. M. & Kioussi, C. 2018 Differential gene regulatory networks in development and disease. Cell Mol Life Sci 75, 1013-1025. (DOI:10.1007/s00018-017-2679-6).
[17] Qin, G., Yang, L., Ma, Y., Liu, J. & Huo, Q. 2019 The exploration of disease-specific gene regulatory networks in esophageal carcinoma and stomach adenocarcinoma. BMC Bioinformatics 20, 717. (DOI:10.1186/s12859-019-3230-6).
[18] Fazilaty, H., Rago, L., Kass Youssef, K., Ocana, O. H., Garcia-Asencio, F., Arcas, A., Galceran, J. & Nieto, M. A. 2019 A gene regulatory network to control EMT programs in development and disease. Nat Commun 10, 5115. (DOI:10.1038/s41467-019-13091-8).
[19] Pezzulo, G. & Levin, M. 2016 Top-down models in biology: explanation and control of complex living systems above the molecular level. J R Soc Interface 13. (DOI:10.1098/rsif.2016.0555).
[20] Pezzulo, G. & Levin, M. 2015 Re-membering the body: applications of computational neuroscience to the top-down control of regeneration of limbs and other complex organs. Integr Biol (Camb) 7, 1487-1517. (DOI:10.1039/c5ib00221d).
[21] Lobo, D., Solano, M., Bubenik, G. A. & Levin, M. 2014 A linear-encoding model explains the variability of the target morphology in regeneration. Journal of the Royal Society, Interface/the Royal Society 11, 20130918. (DOI:10.1098/rsif.2013.0918).
[22] Manicka, S. & Levin, M. 2019 The Cognitive Lens: a primer on conceptual tools for analysing information processing in developmental and regenerative morphogenesis. Philos Trans R Soc Lond B Biol Sci 374, 20180369. (DOI:10.1098/rstb.2018.0369).
[23] Crevier, D. 1993 AI: the tumultuous history of the search for artificial intelligence. New York, NY, BasicBooks; xiv, 386 p. p.
[24] Blackiston, D. J., Vien, K. & Levin, M. 2017 Serotonergic stimulation induces nerve growth and promotes visual learning via posterior eye grafts in a vertebrate model of induced sensory plasticity. npj Regenerative Medicine 2, 8. (DOI:10.1038/s41536-017-0012-5).
[25] Blackiston, D. J. & Levin, M. 2013 Ectopic eyes outside the head in Xenopus tadpoles provide sensory data for light-mediated learning. The Journal of experimental biology 216, 1031-1040. (DOI:10.1242/jeb.074963).
[26] Vandenberg, L. N., Adams, D. S. & Levin, M. 2012 Normalized shape and location of perturbed craniofacial structures in the Xenopus tadpole reveal an innate ability to achieve correct morphology. Developmental Dynamics 241, 863-878. (DOI:10.1002/dvdy.23770).
[27] Harris, A. K. 2018 The need for a concept of shape homeostasis. Biosystems 173, 65-72. (DOI:10.1016/j.biosystems.2018.09.012).
[28] Noble, D. 2012 A theory of biological relativity: no privileged level of causation. Interface Focus 2, 55-64. (DOI: Doi 10.1098/Rsfs.2011.0067).
[29] Noble, D. 2010 Biophysics and systems biology. Philos Trans A Math Phys Eng Sci 368, 1125-1139. (DOI:10.1098/rsta.2009.0245).
[30] Levin, M., Pietak, A. M. & Bischof, J. 2018 Planarian regeneration as a model of anatomical homeostasis: Recent progress in biophysical and computational approaches. Semin Cell Dev Biol 87, 125-144. (DOI:10.1016/j.semcdb.2018.04.003).
[31] Durant, F., Lobo, D., Hammelman, J. & Levin, M. 2016 Physiological controls of large-scale patterning in planarian regeneration: a molecular and computational perspective on growth and form. Regeneration (Oxf) 3, 78-102. (DOI:10.1002/reg2.54).
[32] Durant, F., Morokuma, J., Fields, C., Williams, K., Adams, D. S. & Levin, M.
2017 Long-Term, Stochastic Editing of Regenerative Anatomy via Targeting Endogenous Bioelectric Gradients. Biophysical Journal 112, 2231-2243. (DOI:10.1016/j.bpj.2017.04.011).
[33] Fields, C., Bischof, J. & Levin, M. 2020 Morphological Coordination: A Common Ancestral Function Unifying Neural and Non-Neural Signaling. Physiology (Bethesda) 35, 16-30. (DOI:10.1152/physiol.00027.2019).
[34] Pezzulo, G., Lapalme, J., Durant, F. & Levin, M. 2021 Bistability of Somatic Pattern Memories: Stochastic Outcomes in Bioelectric Circuits Underlying Regeneration. Philosophical Proceedings of the Royal Society B 376, 20190765.
[35] Pezzulo, G. 2020 Disorders of morphogenesis as disorders of inference: Comment on “Morphogenesis as Bayesian inference: A variational approach to pattern formation and control in complex biological systems” by Michael Levin et al. Phys Life Rev. (DOI:10.1016/j.plrev.2020.06.006).
[36] Pezzulo, G. & Levin, M. 2017 Embodying Markov blankets: Comment on “Answering Schrödinger's question: A free-energy formulation” by Maxwell James Désormeau Ramstead et al. Phys Life Rev 24, 32-36. (DOI: doi.org/10.1016/j.plrev.2017.11.020).
[37] Levin, M., Pezzulo, G., and Finkelstein, J. M. 2017 Endogenous Bioelectric Signaling Networks: Exploiting Voltage Gradients for Control of Growth and Form. Annual Review of Biomedical Engineering 19, 353-387. (DOI: DOI:10.1146/annurev-bioeng-071114-040647).
[38] Friston, K., Levin, M., Sengupta, B. & Pezzulo, G. 2015 Knowing one's place: a free-energy approach to pattern regulation. J R Soc Interface 12. (DOI:10.1098/rsif.2014.1383).
[39] Vallverdu, J., Castro, O., Mayne, R., Talanov, M., Levin, M., Baluska, F., Gunji, Y., Dussutour, A., Zenil, H. & Adamatzky, A. 2018 Slime mould: The fundamental mechanisms of biological cognition. Biosystems 165, 57-70. (DOI:10.1016/j.biosystems.2017.12.011).
[40] Fukumoto, T., Kema, I. P. & Levin, M. 2005 Serotonin signaling is a very early step in patterning of the left-right axis in chick and frog embryos. Curr Biol 15, 794-803.
[41] Fukumoto, T., Blakely, R. & Levin, M. 2005 Serotonin transporter function is an early step in left-right patterning in chick and frog embryos. Dev Neurosci 27, 349-363.
[42] Fukumoto, T., Kema, I., Nazarenko, D. & Levin, M. 2003 Serotonin is a novel very early signaling mechanism in left-right asymmetry. Developmental Biology 259, 490a.
[43] Vandenberg, L. N., Lemire, J. M. & Levin, M. 2012 Serotonin has early, cilia-independent roles in Xenopus left-right patterning. Disease models & mechanisms 6, 261-268. (DOI:10.1242/dmm.010256).
[44] Sullivan, K. G. & Levin, M. 2016 Neurotransmitter signaling pathways required for normal development in Xenopus laevis embryos: a pharmacological survey screen. J Anat 229, 483-502. (DOI:10.1111/joa.12467).
[45] Morokuma, J., Blackiston, D. & Levin, M. 2008 KCNQ1 and KCNE1 K+ channel components are involved in early left-right patterning in Xenopus laevis embryos. Cell Physiol Biochem 21, 357-372.
[46] Atsuta, Y., Tomizawa, R. R., Levin, M. & Tabin, C. J. 2019 L-type voltage-gated Ca2+ channel CaV1.2 regulates chondrogenesis during limb development. Proceedings of the National Academy of Sciences, 201908981. (DOI:10.1073/pnas.1908981116).
[47] Pai, V. P., Cervera, J., Mafe, S., Willocq, V., Lederer, E. K. & Levin, M. 2020 HCN2 Channel-Induced Rescue of Brain Teratogenesis via Local and Long-Range Bioelectric Repair. Front Cell Neurosci 14. (DOI:10.3389/fncel.2020.00136).
[48] Mclaughlin, K. A. & Levin, M. 2018 Bioelectric signaling in regeneration: Mechanisms of ionic controls of growth and form. Dev Biol 433, 177-189. (DOI:10.1016/j.ydbio.2017.08.032).
[49] Pitcairn, E., Harris, H., Epiney, J., Pai, V. P., Lemire, J. M., Ye, B., Shi, N. Q., Levin, M. & McLaughlin, K. A. 2017 Coordinating heart morphogenesis: A novel role for Hyperpolarization-activated cyclic nucleotide-gated (HCN) channels during cardiogenesis in Xenopus laevis. Communicative & Biology Integrative 10, e1309488. (DOI:10.1080/19420889.2017.1309488).
[50] Pai, V. P., Willocq, V., Pitcairn, E. J., Lemire, J. M., Pare, J. F., Shi, N. Q., McLaughlin, K. A. & Levin, M. 2017 HCN4 ion channel function is required for early events that regulate anatomical left-right patterning in a nodal and lefty asymmetric gene expression-independent manner. Biology Open 6, 1445-1457. (DOI:10.1242/bio.025957).
[51] Blackiston, D. J., Mclaughlin, K. A. & Levin, M. 2009 Bioelectric controls of cell proliferation: ion channels, membrane voltage and the cell cycle. Cell Cycle 8, 3519-3528.
[52] Heylighen, F. 2013 Self-organization in Communicating Groups: The Emergence of Coordination, Shared References and Collective Intelligence. Complexity Perspectives on Language, Communication and Society, 117-149. (DOI: Book_Doi 10.1007/978-3-642-32817-6).
[53] Deisboeck, T. S. & Couzin, I. D. 2009 Collective behavior in cancer cell populations. BioEssays 31, 190-197. (DOI:10.1002/bies.200800084).
[54] Couzin, I. D. 2009 Collective cognition in animal groups. Trends Cogn Sci 13, 36-43. (DOI: S1364-6613 (08) 00252-0 [pii] 10.1016/j.tics.2008.10.002).
[55] Couzin, I. 2007 Collective minds. Nature 445, 715. (DOI:445715a [pii] 10.1038/445715a).
[56] Pai, V. P., Lemire, J. M., Pare, J. F., Lin, G., Chen, Y. & Levin, M. 2015 Endogenous Gradients of Resting Potential Instructively Pattern Embryonic Neural Tissue via Notch Signaling and Regulation of Proliferation. The Journal of Neuroscience 35, 4366-4385. (DOI:10.1523/JNEUROSCI.1877-14.2015).
[57] Chernet, B. T., Adams, D. S., Lobikin, M. & Levin, M. 2016 Use of genetically encoded, light-gated ion translocators to control tumorigenesis. Oncotarget 7, 19575-19588. (DOI:10.18632/oncotarget.8036).
[58] Chernet, B. T., Fields, C. & Levin, M. 2015 Long-range gap junctional signaling controls oncogene-mediated tumorigenesis in Xenopus laevis embryos. Front Physiol 5, 519. (DOI:10.3389/fphys.2014.00519).
[59] Chernet, B. T. & Levin, M. 2014 Transmembrane voltage potential of somatic cells controls oncogene-mediated tumorigenesis at long-range. Oncotarget 5, 3287-3306.
[60] Chernet, B. T. & Levin, M. 2013 Transmembrane voltage potential is an essential cellular parameter for the detection and control of tumor development in a Xenopus model. Disease models & mechanisms 6, 595-607. (DOI:10.1242/dmm.010835).
[61] Cervera, J., Pietak, A., Levin, M. & Mafe, S. 2018 Bioelectrical coupling in multicellular domains regulated by gap junctions: A conceptual approach. Bioelectrochemistry 123, 45-61. (DOI:10.1016/j.bioelechem.2018.04.013).
[62] Pietak, A. & Levin, M. 2017 Bioelectric gene and reaction networks: computational modelling of genetic, biochemical and bioelectrical dynamics in pattern regulation. J R Soc Interface 14. (DOI:10.1098/rsif.2017.0425).
[63] Pietak, A. & Levin, M. 2016 Exploring Instructive Physiological Signaling with the Bioelectric Tissue Simulation Engine (BETSE). Frontiers in Bioengineering and Biotechnology 4. (DOI:10.3389/fbioe.2016.00055).
[64] Cervera, J., Meseguer, S., Levin, M. & Mafe, S. 2020 Bioelectrical model of head-tail patterning based on cell ion channels and intercellular gap junctions. Bioelectrochemistry 132, 107410. (DOI:10.1016/j.bioelechem.2019.107410).
[65] Cervera, J., Levin, M. & Mafe, S. 2020 Bioelectrical Coupling of Single-Cell States in Multicellular Systems. The Journal of Physical Chemistry Letters, 3234-3241. (DOI:10.1021/acs.jpclett.0c00641).
[66] Cervera, J., Pai, V. P., Levin, M. & Mafe, S. 2019 From non-excitable single-cell to multicellular bioelectrical states supported by ion channels and gap junction proteins: Electrical potentials as distributed controllers. Prog Biophys Mol Biol 149, 39-53. (DOI:10.1016/j.pbiomolbio.2019.06.004).
[67] Cervera, J., Manzanares, J. A., Mafe, S. & Levin, M. 2019 Synchronization of Bioelectric Oscillations in Networks of Nonexcitable Cells: From Single-Cell to Multicellular States. J Phys Chem B 123, 3924-3934. (DOI:10.1021/acs.jpcb.9b01717).
[68] Lobo, D., Malone, T. J. & Levin, M. 2013 Planform: an application and database of graph-encoded planarian regenerative experiments. Bioinformatics. (DOI:10.1093/bioinformatics/btt088).
[69] Lobo, D., Feldman, E. B., Shah, M., Malone, T. J. & Levin, M. 2014 Limbform: a functional ontology-based database of limb regeneration experiments. Bioinformatics 30, 3598-3600. (DOI:10.1093/bioinformatics/btu582).
[70] Lobo, D., Feldman, E. B., Shah, M., Malone, T. J. & Levin, M. 2014 A bioinformatics expert system linking functional data to anatomical outcomes in limb regeneration. Regeneration, n/a-n/a. (DOI:10.1002/reg2.13).
[71] Lobo, D. & Levin, M. 2015 Inferring Regulatory Networks from Experimental Morphological Phenotypes: A Computational Method Reverse-Engineers Planarian Regeneration. PLOS computational biology 11, e1004295. (DOI:10.1371/journal.pcbi.1004295).
[72] Lobo, D., Hammelman, J. & Levin, M. 2016 MoCha: Molecular Characterization of Unknown Pathways. J. Comput. Biol. 23, 291-297. (DOI:10.1089/cmb.2015.0211).
[73] Hammelman, J., Lobo, D. & Levin, M. 2016 Artificial Neural Networks as Models of Robustness in Development and Regeneration: Stability of Memory During Morphological Remodeling. Artificial Neural Network Modelling 628, 45-65. (DOI:10.1007/978-3-319-28495-8_3).
[74] Lobo, D., Morokuma, J. & Levin, M. 2016 Computational discovery and in vivo validation of hnf4 as a regulatory gene in planarian regeneration. Bioinformatics 32, 2681-2685. (DOI:10.1093/bioinformatics/btw299).
[75] Pai, V. P., Pietak, A., Willocq, V., Ye, B., Shi, N. Q. & Levin, M. 2018 HCN2 Rescues brain defects by enforcing endogenous voltage pre-patterns. Nature Communications 9. (DOI:10.1038/s41467-018-03334-5).
[76] Durant, F., Bischof, J., Fields, C., Morokuma, J., LaPalme, J., Hoi, A. & Levin, M. 2019 The Role of Early Bioelectric Signals in the Regeneration of Planarian Anterior/Posterior Polarity. Biophys J 116, 948-961. (DOI:10.1016/j.bpj.2019.01.029).
[77] Kriegman, S., Blackiston, D., Levin, M. & Bongard, J. 2020 A scalable pipeline for designing reconfigurable organisms. Proc Natl Acad Sci USA 117, 1853-1859. (DOI:10.1073/pnas.1910837117).
[78] Tseng, A. S., Beane, W. S., Lemire, J. M., Masi, A. & Levin, M. 2010 Induction of vertebrate regeneration by a transient sodium current. J Neurosci 30, 13192-13200. (DOI:30/39/13192 [pii] 10.1523/JNEUROSCI.3315-10.2010).
[79] Herrera-Rincon, C., Golding, A. S., Moran, K. M., Harrison, C., Martyniuk, C. J., Guay, J. A., Zaltsman, J., Carabello, H., Kaplan, D. L. & Levin, M. 2018 Brief Local Application of Progesterone via a Wearable Bioreactor Induces Long-Term Regenerative Response in Adult Xenopus Hindlimb. Cell Rep 25, 1593-+. (DOI:10.1016/j.celrep.2018.10.010).
[80] Chernet, B. & Levin, M. 2013 Endogenous Voltage Potentials and the Microenvironment: Bioelectric Signals that Reveal, Induce and Normalize Cancer. J Clin Exp Oncol Suppl 1. (DOI:10.4172/2324-9110.S1-002).
[81] Lobikin, M., Chernet, B., Lobo, D. & Levin, M. 2012 Resting potential, oncogene-induced tumorigenesis, and metastasis: the bioelectric basis of cancer in vivo. Physical biology 9, 065002. (DOI:10.1088/1478-3975/9/6/065002).
[82] Rouleau, N., Cairns, D. M., Rusk, W., Levin, M. & Kaplan, D. L. 2021 Learning and synapric plasticity in 3D bioengineered neural tissues. in review.
[83] Rouleau, N., Bonzanni, M., Erndt-Marino, J. D., Sievert, K., Ramirez, C. G., Rusk, W., Levin, M. & Kaplan, D. L. 2020 A 3D Tissue Model of Traumatic Brain Injury with Excitotoxicity That Is Inhibited by Chronic Exposure to Gabapentinoids. Biomolecules 10. (DOI:10.3390/biom10081196).
[84] Bonzanni, M., Rouleau, N., Levin, M. & Kaplan, D. L. 2020 Optogenetically induced cellular habituation in non-neuronal 1 cells. PLOS One 15, e0227230. (DOI:10.1371/journal.pone.0227230).
[85] Bonzanni, M., Payne, S. L., Adelfio, M., Kaplan, D. L., Levin, M. & Oudin, M. J. 2020 Defined extracellular ionic solutions to study and manipulate the cellular resting membrane potential. Biol Open 9. (DOI:10.1242/bio.048553).
[86] Sundelacruz, S., Moody, A. T., Levin, M. & Kaplan, D. L. 2019 Membrane Potential Depolarization Alters Calcium Flux and Phosphate Signaling During Osteogenic Differentiation of Human Mesenchymal Stem Cells. Bioelectricity 1, 56-66. (DOI:10.1089/bioe.2018.0005).
[87] Bonzanni, M., Rouleau, N., Levin, M. & Kaplan, D. L. 2019 On the Generalization of Habituation: How Discrete Biological Systems Respond to Repetitive Stimuli: A Novel Model of Habituation That Is Independent of Any Biological System. BioEssays 41, e1900028. (DOI:10.1002/bies.201900028).
[88] Cairns, D. M., Giordano, J. E., Conte, S., Levin, M. & Kaplan, D. L. 2018 Ivermectin Promotes Peripheral Nerve Regeneration during Wound Healing. ACS Omega 3, 12392-12402. (DOI:10.1021/acsomega.8b01451).
[89] Thurber, A. E., Nelson, M., Frost, C. L., Levin, M., Brackenbury, W. J. & Kaplan, D. L. 2017 IK channel activation increases tumor growth and induces differential behavioral responses in two breast epithelial cell lines. Oncotarget 8, 42382-42397. (DOI:10.18632/oncotarget.16389).
[90] Pai, V. P., Martyniuk, C. J., Echeverri, K., Sundelacruz, S., Kaplan, D. L. & Levin, M. 2016 Genome-wide analysis reveals conserved transcriptional responses downstream of resting potential change in Xenopus embryos, axolotl regeneration, and human mesenchymal cell differentiation. Regeneration (Oxf) 3, 3-25. (DOI:10.1002/reg2.48).
[91] Li, C., Levin, M. & Kaplan, D. L. 2016 Bioelectric modulation of macrophage polarization. Sci Rep 6, 21044. (DOI:10.1038/srep21044).
[92] Sundelacruz, S., Levin, M. & Kaplan, D. L. 2015 Comparison of the depolarization response of human mesenchymal stem cells from different donors. Sci Rep 5, 18279. (DOI:10.1038/srep18279).
[93] Ozkucur, N., Quinn, K. P., Pang, J. C., Du, C., Georgakoudi, I., Miller, E., Levin, M. & Kaplan, D. L. 2015 Membrane potential depolarization causes alterations in neuron arrangement and connectivity in cocultures. Brain Behav 5, 24-38. (DOI:10.1002/brb3.295).
[94] Lobikin, M., Pare, J. F., Kaplan, D. L. & Levin, M. 2015 Selective depolarization of transmembrane potential alters muscle patterning and muscle cell localization in Xenopus laevis embryos. Int J Dev Biol 59, 303-311. (DOI:10.1387/ijdb.150198 ml).
[95] Sundelacruz, S., Li, C., Choi, Y. J., Levin, M. & Kaplan, D. L. 2013 Bioelectric modulation of wound healing in a 3D in vitro model of tissue-engineered bone. Biomaterials 34, 6695-6705. (DOI: S0142-9612 (13) 00616-9 [pii] 10.1016/j.biomaterials.2013.05.040).
[96] Sundelacruz, S., Levin, M. & Kaplan, D. L. 2013 Depolarization alters phenotype, maintains plasticity of predifferentiated mesenchymal stem cells. Tissue engineering. Part A 19, 1889-1908. (DOI:10.1089/ten.tea.2012.0425.rev).
[97] Lan, J.-Y., Williams, C., Levin, M. & Black, L., III. 2014 Depolarization of Cellular Resting Membrane Potential Promotes Neonatal Cardiomyocyte Proliferation In Vitro. Cel. Mol. Bioeng., 1-14. (DOI:10.1007/s12195-014-0346-7).
[98] Blackiston, D., Shomrat, T., Nicolas, C. L., Granata, C. & Levin, M. 2010 A second-generation device for automated training and quantitative behavior analyses of molecularly-tractable model organisms. PLOS One 5, e14370. (DOI:10.1371/journal.pone.0014370).
[99] Shomrat, T. & Levin, M. 2013 An automated training paradigm reveals long-term memory in planarians and its persistence through head regeneration. The Journal of experimental biology 216, 3799-3810. (DOI:10.1242/jeb.087809).
[100] Blackiston, D. J., Anderson, G. M., Rahman, N., Bieck, C. & Levin, M. 2015 A novel method for inducing nerve growth via modulation of host resting potential: gap junction-mediated and serotonergic signaling mechanisms. Neurotherapeutics 12, 170-184. (DOI:10.1007/s13311-014-0317-7).
[101] Manicka, S. & Levin, M. 2019 Modeling somatic computation with non-neural bioelectric networks. Sci Rep 9, 18612. (DOI:10.1038/s41598-019-54859-8).
[102] De Jong, H. 2002 Modeling and simulation of genetic regulatory systems: a literature review. Journal of computational biology 9, 67-103.
[103] Delgado, F. M. & Gómez-Vela, F. 2019 Computational methods for Gene Regulatory Networks reconstruction and analysis: A review. Artificial intelligence in medicine 95, 133-145.
[104] Schlitt, T. & Brazma, A. 2007 Current approaches to gene regulatory network modelling. BMC bioinformatics 8, S9.
[105] Herrera-Delgado, E., Perez-Carrasco, R., Briscoe, J. & Sollich, P. 2018 Memory functions reveal structural properties of gene regulatory networks. PLOS computational biology 14, e1006003. (DOI:10.1371/journal.pcbi.1006003).
[106] Zagorski, M., Tabata, Y., Brandenberg, N., Lutolf, M. P., Tkacik, G., Bollenbach, T., Briscoe, J. & Kicheva, A. 2017 Decoding of position in the developing neural tube from antiparallel morphogen gradients. Science 356, 1379-1383. (DOI:10.1126/science.aam5887).
[107] Szabó, Á., Vattay, G. & Kondor, D. 2012 A cell signaling model as a trainable neural nanonetwork. Nano Communication Networks 3, 57-64.
[108] Turner, C. H., Robling, A. G., Duncan, R. L. & Burr, D. B. 2002 Do bone cells behave like a neuronal network? Calcified Tissue International 70, 435-442.
[109] Goel, P. & Mehta, A. 2013 Learning theories reveal loss of pancreatic electrical connectivity in diabetes as an adaptive response. PLOS One 8, e70366. (DOI:10.1371/journal.pone.0070366).
[110] Nashun, B., Hill, P. W. & Hajkova, P. 2015 Reprogramming of cell fate: epigenetic memory and the erasure of memories past. The EMBO journal 34, 1296-1308. (DOI:10.15252/embj.201490649).
[111] Quintin, J., Cheng, S. C., van der Meer, J. W. & Netea, M. G. 2014 Innate immune memory: towards a better understanding of host defense mechanisms. Curr. Opin. Immunol. 29C, 1-7. (DOI:10.1016/j.coi.2014.02.006).
[112] Corre, G., Stockholm, D., Arnaud, O., Kaneko, G., Vinuelas, J., Yamagata, Y., Neildez-Nguyen, T. M., Kupiec, J. J., Beslon, G., Gandrillon, O., et al. 2014 Stochastic fluctuations and distributed control of gene expression impact cellular memory. PLOS One 9, e115574. (DOI:10.1371/journal.pone.0115574).
[113] Zediak, V. P., Wherry, E. J. & Berger, S. L. 2011 The contribution of epigenetic memory to immunologic memory. Curr Opin Genet Dev 21, 154-159. (DOI:10.1016/j.gde.2011.01.016).
[114] Watson, R. A., Buckley, C. L., Mills, R. & Davies, A. 2010 Associative memory in gene regulation networks. In Artificial Life Conference XII (pp. 194-201. Odense, Denmark.
[115] Watson, R. A., Mills, R. & Buckley, C. L. 2011 Global adaptation in networks of selfish components: emergent associative memory at the system scale. Artif. Life 17, 147-166. (DOI:10.1162/artl_a_00029).
[116] Science, A. A. f. t. A. o. 2003 Maturing from Memory. Science Signaling 2003, tw462-tw462.
[117] Sible, J. C. 2003 Thanks for the memory. Nature 426, 392-393.
[118] Xiong, W. & Ferrell, J. E. 2003 A positive-feedback-based bistable ‘memory module’ that governs a cell fate decision. Nature 426, 460-465.
[119] Levine, J. H., Lin, Y. & Elowitz, M. B. 2013 Functional roles of pulsing in genetic circuits. Science 342, 1193-1200.
[120] Urrios, A., Macia, J., Manzoni, R., Conde, N., Bonforti, A., de Nadal, E. l., Posas, F. & Solé, R. 2016 A synthetic multicellular memory device. ACS synthetic biology 5, 862-873.
[121] Macia, J., Vidiella, B. & Solé, R. V. 2017 Synthetic associative learning in engineered multicellular consortia. Journal of The Royal Society Interface 14, 20170158.
[122] Kandel, E. R., Dudai, Y. & Mayford, M. R. 2014 The molecular and systems biology of memory. Cell 157, 163-186.
[123] Ryan, T. J., Roy, D. S., Pignatelli, M., Arons, A. & Tonegawa, S. 2015 Engram cells retain memory under retrograde amnesia. Science 348, 1007-1013.
[124] Szilagyi, A., Szabo, P., Santos, M. & Szathmary, E. 2020 Phenotypes to remember: Evolutionary developmental memory capacity and robustness. PLOS computational biology 16, e1008425. (DOI:10.1371/journal.pcbi.1008425).
[125] Palm, G. 1980 On associative memory. Biological cybernetics 36, 19-31.
[126] Kohonen, T. 2012 Self-organization and associative memory, Springer Science & Business Media.
[127] Rescorla, R. A. 1967 Pavlovian conditioning and its proper control procedures. Psychological review 74, 71.
[128] Lee, T. I. & Young, R. A. 2013 Transcriptional regulation and its misregulation in disease. Cell 152, 1237-1251. (DOI:10.1016/j.cell.2013.02.014).
[129] Fernando, C. T., Liekens, A. M. L., Bingle, L. E. H., Beck, C., Lenser, T., Stekel, D. J. & Rowe, J. E. 2009 Molecular circuits for associative learning in single-celled organisms. Journal of the Royal Society Interface 6, 463-469. (DOI:10.1098/rsif.2008.0344).
[130] McGregor, S., Vasas, V., Husbands, P. & Fernando, C. 2012 Evolution of associative learning in chemical networks. PLOS computational biology 8, e1002739. (DOI:10.1371/journal.pcbi.1002739).
[131] Gantt, W. H. 1981 Organ-system responsibility, schizokinesis, and autokinesis in behavior. Pavlov J Biol Sci 16, 64-66.
[132] Gantt, W. H. 1974 Autokinesis, schizokinesis, centrokinesis and organ-system responsibility: concepts and definition. Pavlov J Biol Sci 9, 187-191.
[133] Frey, N., Bodmer, M., Bircher, A., Jick, S. S., Meier, C. R. & Spoendlin, J. 2019 Stevens-Johnson Syndrome and Toxic Epidermal Necrolysis in Association with Commonly Prescribed Drugs in Outpatient Care Other than Anti-Epileptic Drugs and Antibiotics: A Population-Based Case-Control Study. Drug Saf 42, 55-66. (DOI:10.1007/s40264-018-0711-x).
[134] Kauffman, S. A. 1969 Metabolic stability and epigenesis in randomly constructed genetic nets. Journal of theoretical biology 22, 437-467.
[135] Thomas, R. 1973 Boolean formalization of genetic control circuits. Journal of theoretical biology 42, 563-585.
[136] Kauffman, S. A., and Richard C. Strohman. 1994 The Origins of Order: self organization and selection in evolution. New York, Oxford university press.
[137] Saez-Rodriguez, J., Alexopoulos, L. G., Epperlein, J., Samaga, R., Lauffenburger, D. A., Klamt, S. & Sorger, P. K. 2009 Discrete logic modelling as a means to link protein signalling networks with functional analysis of mammalian signal transduction. Molecular systems biology 5, 331.
[138] Marques-Pita, M. & Rocha, L. M. 2013 Canalization and control in automata networks: body segmentation in Drosophila melanogaster. PLOS One 8, e55946. (DOI:10.1371/journal.pone.0055946).
[139] Zanudo, J. G. & Albert, R. 2015 Cell fate reprogramming by control of intracellular network dynamics. PLOS computational biology 11, e1004193. (DOI:10.1371/journal.pcbi.1004193).
[140] Eduati, F., Doldàn-Martelli, V., Klinger, B., Cokelaer, T., Sieber, A., Kogera, F., Dorel, M., Garnett, M. J., Blüthgen, N. & Saez-Rodriguez, J. 2017 Drug resistance mechanisms in colorectal cancer dissected with cell type-specific dynamic logic models. Cancer research 77, 3364-3375.
[141] Demongeot, J., Hasgui, H. & Thellier, M. 2019 Memory in plants: Boolean modeling of the learning and store/recall memory functions in response to environmental stimuli. Journal of theoretical biology 467, 123-133.
[142] Helikar, T., Kowal, B., McClenathan, S., Bruckner, M., Rowley, T., Madrahimov, A., Wicks, B., Shrestha, M., Limbu, K. & Rogers, J. A. 2012 The cell collective: toward an open and collaborative approach to systems biology. BMC systems biology 6, 96.
[143] Albert, I., Thakar, J., Li, S., Zhang, R. & Albert, R. 2008 Boolean network simulations for life scientists. Source Code Biol Med 3, 16. (DOI:10.1186/1751-0473-3-16).
[144] Albert, R. & Thakar, J. 2014 Boolean modeling: a logic-based dynamic approach for understanding signaling and regulatory networks and for making useful predictions. Wiley Interdisciplinary Reviews: Systems Biology and Medicine 6, 353-369. (DOI:10.1002/wsbm.1273).
[145] Albert, R. e. 2004 Boolean Modeling of Genetic Regulatory Networks. In Complex Networks. Lecture Notes in Physics (ed. F. H. Ben-Naim E., Toroczkai Z.). Berlin, Heidelberg, Springer.
[146] Wang, R. S., Saadatpour, A. & Albert, R. 2012 Boolean modeling in systems biology: an overview of methodology and applications. Phys Biol 9, 055001. (DOI:10.1088/1478-3975/9/5/055001).
[147] Banerjee, K. 2015 Dynamic memory of a single voltage-gated potassium ion channel: A stochastic nonequilibrium thermodynamic analysis. J. Chem. Phys. 142, 185101. (DOI:10.1063/1.4920937).
[148] Debanne, D., Daoudal, G., Sourdet, V. & Russier, M. 2003 Brain plasticity and ion channels. J. Physiol. Paris 97, 403-414. (DOI:10.1016/j.jphysparis.2004.01.004).
[149] Daoudal, G. & Debanne, D. 2003 Long-term plasticity of intrinsic excitability: learning rules and mechanisms. Learning & memory 10, 456-465. (DOI:10.1101/lm.64103).
[150] Gallaher, J., Bier, M. & van Heukelom, J. S. 2010 First order phase transition and hysteresis in a cell's maintenance of the membrane potential-An essential role for the inward potassium rectifiers. Biosystems 101, 149-155. (DOI: S0303-2647 (10) 00095-X [pii] 10.1016/j.biosystems.2010.05.007).
[151] Geukes Foppen, R. J., van Mil, H. G. & van Heukelom, J. S. 2002 Effects of chloride transport on bistable behaviour of the membrane potential in mouse skeletal muscle. The Journal of physiology 542, 181-191.
[152] Izquierdo, E. J., Williams, P. L. & Beer, R. D. 2015 Information Flow through a Model of the C. elegans Klinotaxis Circuit. PLOS One 10, e0140397. (DOI:10.1371/journal.pone.0140397).
[153] Law, R. & Levin, M. 2015 Bioelectric memory: modeling resting potential bistability in amphibian embryos and mammalian cells. Theor Biol Med Model 12, 22. (DOI:10.1186/s12976-015-0019-9).
[154] Snipas, M., Kraujalis, T., Paulauskas, N., Maciunas, K. & Bukauskas, F. F. 2016 Stochastic Model of Gap Junctions Exhibiting Rectification and Multiple Closed States of Slow Gates. Biophys J 110, 1322-1333. (DOI:10.1016/j.bpj.2016.01.035).
[155] Stockwell, S. R., Landry, C. R. & Rifkin, S. A. 2015 The yeast galactose network as a quantitative model for cellular memory. Mol Biosyst 11, 28-37. (DOI:10.1039/c4mb00448e).
[156] Yamauchi, B. & Beer, R. 1994 Integrating Reactive, Sequential, and Learning-Behavior Using Dynamical Neural Networks. Com Adap Sy, 382-391.
[157] Tagkopoulos, I., Liu, Y. C. & Tavazoie, S. 2008 Predictive behavior within microbial genetic networks. Science 320, 1313-1317. (DOI:10.1126/science.1154456).
[158] Fernando, C. T., Liekens, A. M., Bingle, L. E., Beck, C., Lenser, T., Stekel, D. J. & Rowe, J. E. 2009 Molecular circuits for associative learning in single-celled organisms. J R Soc Interface 6, 463-469. (DOI:10.1098/rsif.2008.0344).
[159] Deritei, D., Rozum, J., Regan, E. R. & Albert, R. 2019 A feedback loop of conditionally stable circuits drives the cell cycle from checkpoint to checkpoint. Scientific reports 9, 1-19.
[160] Zañudo, J. G. T., Yang, G. & Albert, R. 2017 Structure-based control of complex networks with nonlinear dynamics. Proceedings of the National Academy of Sciences 114, 7234-7239.
[161] Sherrington, D. & Wong, K. 1989 Random boolean networks for autoassociative memory. Physics reports 184, 293-299.
[162] Sherrington, D. & Wong, K. 1990 Random Boolean networks for autoassociative memory: Optimization and sequential learning. In Statistical Mechanics of Neural Networks (pp. 467-473, Springer.
[163] Sparkes, A., Aubrey, W., Byrne, E., Clare, A., Khan, M. N., Liakata, M., Markham, M., Rowland, J., Soldatova, L. N., Whelan, K. E., et al. 2010 Towards Robot Scientists for autonomous scientific discovery. Autom Exp 2, 1. (DOI:10.1186/1759-4499-2-1).
[164] Qi, D., King, R. D., Hopkins, A. L., Bickerton, G. R. & Soldatova, L. N. 2010 An ontology for description of drug discovery investigations. J Integr Bioinform 7. (DOI:10.2390/biecoll-jib-2010-126 472 [pii]).
[165] King, R. D., Rowland, J., Oliver, S. G., Young, M., Aubrey, W., Byrne, E., Liakata, M., Markham, M., Pir, P., Soldatova, L. N., et al. 2009 The automation of science. Science 324, 85-89. (DOI:324/5923/85 [pii] 10.1126/science.1165620).
[166] Soldatova, L. N., Clare, A., Sparkes, A. & King, R. D. 2006 An ontology for a Robot Scientist. Bioinformatics 22, e464-471. (DOI:22/14/e464 [pii] 10.1093/bioinformatics/bt1207).
[167] Lobo, D., Lobikin, M. & Levin, M. 2017 Discovering novel phenotypes with automatically inferred dynamic models: a partial melanocyte conversion in Xenopus. Sci Rep 7, 41339. (DOI:10.1038/srep41339).
[168] Levin, M. 1998 Matrix-based GA representations in a model of the evolution of communication. In Applications Handbook of Genetic Algorithms (pp. 103-117. Boca Raton, FL, CRC Press.
[169] Levin, M. 1995 The evolution of understanding: A genetic algorithm model of the evolution of communication. Biosystems 36, 167-178.
[170] Levin, M. 1995 Use of Genetic Algorithms to Solve Biomedical Problems. M D Comput. 12, 193-199.
[171] Levin, M. 1995 Locating putative protein signal sequences using genetic algorithms. In Applications Handbook of Genetic Algorithms (pp. 53-66. Boca Raton, FL, CRC Press.
[172] Fava, G. A. 2020 May antidepressant drugs worsen the conditions they are supposed to treat? The clinical foundations of the oppositional model of tolerance. Ther Adv Psychopharmacol 10, 2045125320970325. (DOI:10.1177/2045125320970325).
[173] Fava, G. A. & Offidani, E. 2011 The mechanisms of tolerance in antidepressant action. Prog Neuropsychopharmacol Biol Psychiatry 35, 1593-1602. (DOI:10.1016/j.pnpbp.2010.07.026).
[174] Revusky, S., Taukulis, H. K. & Peddle, C. 1979 Learned Associations between Drug States-Attempted Analysis in Pavlovian Terms. Physiological Psychology 7, 352-363.
[175] Remy, S. & Beck, H. 2006 Molecular and cellular mechanisms of pharmacoresistance in epilepsy. Brain 129, 18-35. (DOI:10.1093/brain/awh682).
[176] Deshpande, L. S., Blair, R. E., Nagarkatti, N., Sombati, S., Martin, B. R. & DeLorenzo, R. J. 2007 Development of pharmacoresistance to benzodiazepines but not cannabinoids in the hippocampal neuronal culture model of status epilepticus. Exp Neurol 204, 705-713. (DOI:10.1016/j.expneurol.2007.01.001).
[177] Azad, A. K., Lawen, A. & Keith, J. M. 2015 Prediction of signaling cross-talks contributing to acquired drug resistance in breast cancer cells by Bayesian statistical modeling. BMC Syst Biol 9, 2. (DOI:10.1186/s12918-014-0135-x).
[178] Wilson, M. Z., Ravindran, P. T., Lim, W. A. & Toettcher, J. E. 2017 Tracing Information Flow from Erk to Target Gene Induction Reveals Mechanisms of Dynamic and Combinatorial Control. Mol Cell 67, 757-769 e755. (DOI:10.1016/j.molcel.2017.07.016).
[179] Liu, P., Kevrekidis, I. G. & Shvartsman, S. Y. 2011 Substrate-dependent control of ERK phosphorylation can lead to oscillations. Biophys J 101, 2572-2581. (DOI:10.1016/j.bpj.2011.10.025).
[180] Davidich, M. I. & Bornholdt, S. 2008 Boolean network model predicts cell cycle sequence of fission yeast. PLOS One 3, e1672. (DOI:10.1371/journal.pone.0001672).
[181] Kim, J., Park, S. M. & Cho, K. H. 2013 Discovery of a kernel for controlling biomolecular regulatory networks. Sci Rep 3, 2223. (DOI:10.1038/srep02223).
[182] Abou-Jaoude, W., Traynard, P., Monteiro, P. T., Saez-Rodriguez, J., Helikar, T., Thieffry, D. & Chaouiya, C. 2016 Logical Modeling and Dynamical Analysis of Cellular Networks. Front Genet 7, 94. (DOI:10.3389/fgene.2016.00094).
[183] Abou-Jaoude, W., Thieffry, D. & Feret, J. 2016 Formal derivation of qualitative dynamical models from biochemical networks. Biosystems 149, 70-112. (DOI:10.1016/j.biosystems.2016.09.001).
[184] Shankaran, H., Wiley, H. S. & Resat, H. 2007 Receptor downregulation and desensitization enhance the information processing ability of signalling receptors. BMC Syst Biol 1, 48. (DOI:10.1186/1752-0509-1-48).
[185] Schmidhuber, J. 2020 Generative Adversarial Networks are special cases of Artificial Curiosity (1990) and also closely related to Predictability Minimization (1991). Neural Netw 127, 58-66. (DOI:10.1016/j.neunet.2020.04.008).
[186] Williams, K., Bischof, J., Lee, F., Miller, K., LaPalme, J., Wolfe, B. & Levin, M. 2020 Regulation of axial and head patterning during planarian regeneration by a commensal bacterium. Mech Dev, 103614. (DOI:10.1016/j.mod.2020.103614).
[187] Lee, F. J., Williams, K. B., Levin, M. & Wolfe, B. E. 2018 The Bacterial Metabolite Indole Inhibits Regeneration of the Planarian Flatworm Dugesia japonica. iScience 10, 135-148. (DOI:10.1016/j.isci.2018.11.021).
[188] Westerhoff, H. V., Brooks, A. N., Simeonidis, E., Garcia-Contreras, R., He, F., Boogerd, F. C., Jackson, V. J., Goncharuk, V. & Kolodkin, A. 2014 Macromolecular networks and intelligence in microorganisms. Front Microbiol 5, 379. (DOI:10.3389/fmicb.2014.00379).
[189] Gallistel, C. R. & Balsam, P. D. 2014 Time to rethink the neural mechanisms of learning and memory. Neurobiol. Learn. Mem. 108, 136-144. (DOI:10.1016/j.nlm.2013.11.019).
[190] Nechansky, H. 2013 Elements of a cybernetic epistemology: complex anticipatory systems. Kybernetes 42, 207-225. (DOI:10.1108/03684921311310576).
[191] Nechansky, H. 2013 Elements of a cybernetic epistemology: elementary anticipatory systems. Kybernetes 42, 185-206. (DOI:10.1108/03684921311310567).
[192] Dhar, R., Sagesser, R., Weikert, C. & Wagner, A. 2013 Yeast adapts to a changing stressful environment by evolving cross-protection and anticipatory gene regulation. Mol Biol Evol 30, 573-588. (DOI:10.1093/molbev/mss253).
[193] Mossbridge, J., Tressoldi, P. & Utts, J. 2012 Predictive physiological anticipation preceding seemingly unpredictable stimuli: a meta-analysis. Front Psychol 3, 390. (DOI:10.3389/fpsyg.2012.00390).
[194] Qu, F., Qiao, Q., Wang, N., Ji, G., Zhao, H., He, L., Wang, H. & Bao, G. 2016 Genetic polymorphisms in circadian negative feedback regulation genes predict overall survival and response to chemotherapy in gastric cancer patients. Sci Rep 6, 22424. (DOI:10.1038/srep22424).
[195] Papagiannakopoulos, T., Bauer, M. R., Davidson, S. M., Heimann, M., Subbaraj, L., Bhutkar, A., Bartlebaugh, J., Vander Heiden, M. G. & Jacks, T. 2016 Circadian Rhythm Disruption Promotes Lung Tumorigenesis. Cell Metab 24, 324-331. (DOI:10.1016/j.cmet.2016.07.001).
[196] Masri, S., Papagiannakopoulos, T., Kinouchi, K., Liu, Y., Cervantes, M., Baldi, P., Jacks, T. & Sassone-Corsi, P. 2016 Lung Adenocarcinoma Distally Rewires Hepatic Circadian Homeostasis. Cell 165, 896-909. (DOI:10.1016/j.cell.2016.04.039).
[197] Sancar, A., Lindsey-Boltz, L. A., Gaddameedhi, S., Selby, C. P., Ye, R., Chiou, Y. Y., Kemp, M. G., Hu, J., Lee, J. H. & Ozturk, N. 2015 Circadian clock, cancer, and chemotherapy. Biochemistry 54, 110-123. (DOI:10.1021/bi5007354).
[198] Wood, P. A., Yang, X. & Hrushesky, W. J. 2009 Clock genes and cancer. Integr Cancer Ther 8, 303-308. (DOI:10.1177/1534735409355292).
[199] Hrushesky, W. J., Grutsch, J., Wood, P., Yang, X., Oh, E. Y., Ansell, C., Kidder, S., Ferrans, C., Quiton, D. F., Reynolds, J., et al. 2009 Circadian clock manipulation for cancer prevention and control and the relief of cancer symptoms. Integr Cancer Ther 8, 387-397. (DOI:10.1177/1534735409352086).
[200] Herrera-Rincon, C., Pare, J. F., Martyniuk, C. J., Jannetty, S. K., Harrison, C., Fischer, A., Dinis, A., Keshari, V., Novak, R. & Levin, M. 2020 An in vivo brain-bacteria interface: the developing brain as a key regulator of innate immunity. NPJ Regen Med 5, 2. (DOI:10.1038/s41536-020-0087-2).
[201] Pare, J. F., Martyniuk, C. J. & Levin, M. 2017 Bioelectric regulation of innate immune system function in regenerating and intact Xenopus laevis. Npj Regenerative Medicine 2, 15-. (DOI: UNSP 15 10.1038/s41536-017-0019-y).
[202] Liaudanskaya, V., Chung, J. Y., Mizzoni, C., Rouleau, N., Berk, A. N., Wu, L., Turner, J. A., Georgakoudi, I., Whalen, M. J., Nieland, T. J. F., et al. 2020 Modeling Controlled Cortical Impact Injury in 3D Brain-Like Tissue Cultures. Adv Healthc Mater 9, e2000122. (DOI:10.1002/adhm.202000122).
[203] Liaudanskaya, V., Jgamadze, D., Berk, A. N., Bischoff, D. J., Gu, B. J., Hawks-Mayer, H., Whalen, M. J., Chen, H. I. & Kaplan, D. L. 2019 Engineering advanced neural tissue constructs to mitigate acute cerebral inflammation after brain transplantation in rats. Biomaterials 192, 510-522. (DOI:10.1016/j.biomaterials.2018.11.031).
[204] Cantley, W. L., Du, C., Lomoio, S., DePalma, T., Peirent, E., Kleinknecht, D., Hunter, M., Tang-Schomer, M. D., Tesco, G. & Kaplan, D. L. 2018 Functional and Sustainable 3D Human Neural Network Models from Pluripotent Stem Cells. Acs Biomaterials Science & Engineering 4, 4278-4288. (DOI:10.1021/acsbiomaterials.8b00622).
[205] Norman, T. M., Lord, N. D., Paulsson, J. & Losick, R. 2013 Memory and modularity in cell-fate decision making. Nature 503, 481-486. (DOI:10.1038/nature12804).
[206] Ball, P. 2008 Cellular memory hints at the origins of intelligence. Nature 451, 385. (DOI:10.1038/451385a).
[207] Spencer, G. J. & Genever, P. G. 2003 Long-term potentiation in bone—a role for glutamate in strain-induced cellular memory? BMC cell biology 4, 9. (DOI:10.1186/1471-2121 Apr. 9).
[208] Sparkman, N. L. & Li, M. 2012 Drug-drug conditioning between citalopram and haloperidol or olanzapine in a conditioned avoidance response model: implications for polypharmacy in schizophrenia. Behav. Pharmacol. 23, 658-668. (DOI:10.1097/FBP.0b013e328358590d).
[209] Revusky, S. 1982 The Drug-Drug Conditioning Paradigm—a Review. Psychopharmacology 76, A11-A11.
[210] Taukulis, H. K. & Brake, L. D. 1989 Therapeutic and Hypothermic Properties of Diazepam Altered by a Diazepam-Chlorpromazine Association. Pharmacology Biochemistry and Behavior 34, 1-6. (DOI: Doi 10.1016/0091-3057 (89) 90343-2).
[211] Yoney, A., Etoc, F., Ruzo, A., Carroll, T., Metzger, J. J., Martyn, I., Li, S., Kirst, C., Siggia, E. D. & Brivanlou, A. H. 2018 WNT signaling memory is required for ACTIVIN to function as a morphogen in human gastruloids. Elife 7. (DOI:10.7554/eLife.38279).
[212] Bugaj, L. J., Sabnis, A. J., Mitchell, A., Garbarino, J. E., Toettcher, J. E., Bivona, T. G. & Lim, W. A. 2018 Cancer mutations and targeted drugs can disrupt dynamic signal encoding by the Ras-Erk pathway. Science 361. (DOI:10.1126/science.aao3048).
[213] Crommelinck, M., Feltz, B. & Goujon, P. 2006 Self-organization and emergence in life sciences, Springer.
[214] Karsenti, E. 2008 Self-organization in cell biology: a brief history. Nature reviews Molecular cell biology 9, 255-262.
[215] Levin, M. 2014 Endogenous bioelectrical networks store non-genetic patterning information during development and regeneration. The Journal of Physiology 592, 2295-2305. (DOI:10.1113/jphysiol.2014.271940).
[216] Emmons-Bell, M., Durant, F., Tung, A., Pietak, A., Miller, K., Kane, A., Martyniuk, C. J., Davidian, D., Morokuma, J. & Levin, M. 2019 Regenerative Adaptation To Electrochemical Perturbation In Planaria: A Molecular Analysis Of Physiological Plasticity. iScience in press. (DOI:10.1016/j.isci.2019.11.014).
[217] Sullivan, K. G., Emmons-Bell, M. & Levin, M. 2016 Physiological inputs regulate species-specific anatomy during embryogenesis and regeneration. Commun Integr Biol 9, e1192733. (DOI:10.1080/19420889.2016.1192733).
[218] Schreier, H. I., Soen, Y. & Brenner, N. 2017 Exploratory adaptation in large random networks. Nat Commun 8, 14826. (DOI:10.1038/ncomms14826).
[219] Soen, Y., Knafo, M. & Elgart, M. 2015 A principle of organization which facilitates broad Lamarckian-like adaptations by improvisation. Biol Direct 10, 68. (DOI:10.1186/s13062-015-0097-y).
[220] Watson, R. A., Wagner, G. P., Pavlicev, M., Weinreich, D. M. & Mills, R. 2014 The evolution of phenotypic correlations and “developmental memory”. Evolution 68, 1124-1138. (DOI:10.1111/evo.12337).
[221] Sorek, M., Balaban, N. Q. & Loewenstein, Y. 2013 Stochasticity, bistability and the wisdom of crowds: a model for associative learning in genetic regulatory networks. PLOS computational biology 9, e1003179. (DOI:10.1371/journal.pcbi.1003179).
[222] Manicka, S. & Harvey, I. 2008 ‘Psychoanalysis’ of a Minimal Agent. In Artificial Life XI (
[223] Crutchfield, J. P., Mitchell, M. & Das, R. 1998 The Evolutionary Design of Collective Computation in Cellular Automata. In arXiv e-prints

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating a disease or disorder characterized by a gene regulatory network (GRN) and a therapeutic agent in a subject in need thereof, the method comprising as steps:
(i) administering to the subject the therapeutic agent and an inert agent, wherein the therapeutic agent triggers a response in the GRN and a corresponding therapeutic response in the subject when the therapeutic agent is administered to the subject without the inert agent, and wherein the inert agent does not trigger a response in the GRN and a corresponding therapeutic response in the subject when the inert agent is administered to the subject without the therapeutic agent; and
(ii) subsequently repeating step (i) one or more times until the inert agent triggers a response in the GRN and a corresponding therapeutic response in the subject when the inert agent is administered to the subject without the therapeutic agent, thereby treating the disease or disorder characterized by the GRN in the subject by administering the inert agent.

2. The method of claim 1, further comprising the step of (iii) administering to the subject the inert agent until the inert agent ceases to trigger a response in the GRN and a corresponding therapeutic response in the subject.

3. The method of claim 1, wherein the GRN is selected from the group consisting of “Aurora Kinase A in Neuroblastoma,” “CD4+ T Cell Differentiation and Plasticity,” “Human Gonadal Sex Determination,” “B cell differentiation,” and “Fanconi Anemia and Checkpoint Recovery.”

4. The method of claim 1, wherein the disease or disorder is comprises at least one of cancer, a metabolic disease or disorder, or a developmental disease or disorder.

5-7. (canceled)

8. The method of claim 1, wherein the inert agent is fluid flow.

9. (canceled)

10. A method for treating a disease or disorder characterized by a gene regulatory network (GRN) and a standard dose of therapeutic agent in a subject in need thereof, the method comprising as steps:
(i) administering to the subject the standard dose of the therapeutic agent and a dose of an inert agent, wherein the standard dose of the therapeutic agent triggers a response in the GRN and a corresponding therapeutic response in the subject when the dose of therapeutic agent is administered to the subject without the dose of the inert agent, and wherein the dose of the inert agent does not trigger a response in the GRN and a corresponding therapeutic response when the dose of the inert agent is administered to the subject without the standard dose of the therapeutic agent; and
(ii) subsequently repeating step (i) one or more times until the dose of the inert agent triggers a response in the GRN and a corresponding therapeutic response when the dose of the inert agent is administered to the subject without the dose of the therapeutic agent, thereby treating the disease or disorder characterized by the GRN in the subject by administering the dose of the inert agent.

11. The method of claim 10, further comprising as a step (iii) continuing to administer the dose of the inert agent to the subject until the dose of the inert agent ceases to trigger a response in the GRN and a corresponding therapeutic response in the subject.

12. The method of claim 10, wherein the standard dose of the therapeutic agent triggers undesirable side effects in the subject and the method further comprises as a step (iii) subsequently administering to the subject a lower dose of the therapeutic agent than the standard dose of the therapeutic agent and optionally a dose of the inert agent, wherein the subsequently administered lower dose of the therapeutic agent triggers a response in the GRN and a corresponding therapeutic response in the subject without triggering side effects in the subject or triggering reduces side effects in the subject.

13. The method of claim 12, further comprising as a step (iv) continuing to administer the lower dose of the therapeutic agent to the subject until the lower dose of the therapeutic agent ceases to trigger a response in the GRN and a corresponding therapeutic effect in the subject.

14. The method of claim 10, wherein the GRN is selected from the group consisting of “Aurora Kinase A in Neuroblastoma,” “CD4+ T Cell Differentiation and Plasticity,” “Human Gonadal Sex Determination,” “B cell differentiation,” and “Fanconi Anemia and Checkpoint Recovery.”

15. (canceled)

16. The method of claim 10, wherein the inert agent is fluid flow.

17. (canceled)

18. The method of claim 10, wherein the disease or disorder comprises at least one of cancer, a metabolic disease or disorder, or a developmental disease or disorder.

19-20. (canceled)

21. A system for determining whether a determining whether a gene regulatory network (GRN) exhibits memory, the system comprising at least one hardware processor that is programmed to perform one or more of the following steps:
(A) simulating administering to the GRN an unconditioned stimuli (UCS) and determining whether the UCS triggers a response by the GRN; and
(i) if the UCS does not trigger a response by the GRN, then repeating step (A) using another different UCS until the UCS triggers a response by the GRN; or
(ii) if/when the UCS triggers a response by the GRN, then allowing the GRN to relax and simulating administering the UCS to the GRN and determining whether the GRN exhibits UCS-based memory (UM), and if the GRN does not exhibit UM then proceeding to step (B) or if the GRN exhibits UM then optionally completing the method;
(B) simulating administering to the GRN a combination of an unconditioned stimulus (UCS) and a neutral stimulus (NS) and determining whether the combination of the UCS and the NS triggers a response by the GRN; and
(i) if the combination of the UCS and the NS does not trigger a response by the GRN, then repeating step (B) using a combination of the UCS and another different NS until the combination of the UCS and the NS triggers a response by the GRN; or
(ii) if/when the combination of the UCS and the NS triggers a response by the GRN, then allowing the GRN to relax and simulating administering the combination of the UCS and the NS and determining whether the GRN exhibits pairing memory (PM), and if the GRN does not exhibit PM then proceeding to step (C) or step (D) and if the GRN exhibits PM then optionally completing the method;
(C) simulating administering to the GRN an unconditioned stimuli (UCS) and determining whether the UCS triggers a response by the GRN; and
(i) if the UCS does not trigger a response by the GRN, then repeating step (C) using another different UCS until the UCS triggers a response by the GRN; and
(ii) if/when the UCS triggers a response by the GRN, then allowing the GRN to relax and simulating administering a NS to the GRN and determining whether the GRN exhibits transfer memory (TM), and if the GRN does not exhibit TM then proceeding to step (D) or if the GRN exhibits TM then optionally completing the method;
(D) simulating administering to the GRN a combination of an unconditioned stimulus (UCS) and a neutral stimulus (NS) and determining whether the combination of the UCS and the NS triggers a response by the GRN; and
(i) if the combination of the UCS and the NS does not trigger a response by the GRN, then repeating step (D) using a different combination of another different UCS and/or another different NS until the combination of the UCS and the NS triggers a response by the GRN; or
(ii) if/when the combination of the UCS and the NS triggers a response by the GRN, then allowing the GRN to relax and simulating administering the NS and determining whether the GRN exhibits associative memory (AM), and if the GRN does not exhibit AM then proceeding to step (E), or if the GRN does exhibit AM then optionally completing the method or optionally proceeding to step (D) (iii); or
(iii) if the GRN exhibits AM, then allowing the GRN to relax and simulating administering the NS and determining whether the GRN exhibits long recall AM (LRAM), and if the GRN does not exhibit LRAM, then determining that the GRN exhibits short recall AM (SRAM) and optionally repeating step (D) using a different combination of another different UCS and/or another different NS until the GRN exhibits LRAM; and
(E) after performing step (D), allowing the GRN to relax and simulating administering the NS and determining whether the GRN exhibits consolidation memory (CM), and if the GRN exhibits CM optionally completing the method or if the GRN does not exhibit CM then determining that the GRN does not exhibit memory.

22. The system of claim 21 further comprising software for programming the hardware processor to perform one or more of steps (A), (B), (C), (D), and (E).

23. (canceled)
